SEQUENCE LISTING (62 sequences)

SEQ ID NO: 1 - DNA, 49 bp, Artificial Sequence
  Nucleotide sequence of alphavirus native subgenomic promoter
ctctctacggctaacctgaatggactacgacatagtctagtccgccaag

SEQ ID NO: 2 - DNA, 61 bp, Artificial Sequence
  Nucleotide sequence of extended subgenomic promoter (v2)
gggcccctataactctctacggctaacctgaatggactacgacatagtctagtccgccaag

SEQ ID NO: 3 - DNA, 80 bp, Artificial Sequence
  Nucleotide sequence of extended subgenomic promoter (v3)
tcattcagctacctgagaggggcccctataactctctacggctaacctgaatggactacgacatagtctagtccgccaag

SEQ ID NO: 4 - DNA, 575 bp, Artificial Sequence
  Nucleotide sequence of wild-type EMCV IRES
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 5 - DNA, 1692 bp, Artificial Sequence
  Nucleotide sequence of influenza A virus H5 hemagglutinin subtype
atggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaa

SEQ ID NO: 6 - DNA, 1350 bp, Artificial Sequence
  Nucleotide sequence of influenza A virus N1 neuraminidase subtype
atgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtag

SEQ ID NO: 7 - DNA, 1260 bp, Artificial Sequence
  Nucleotide sequence of SARS-CoV-2 nucleocapsid (N) protein
atgagcgacaacggacctcagaaccagagaaatgcccctagaatcacctttggcggacctagcgacagcaccggcagcaaccagaatggcgagagaagcggcgccagatctaagcagcggcgtccacagggactgcccaacaacaccgccagctggttcaccgccctcacccagcacggcaaagaggacctgaagttcccccggggacagggcgtgccaatcaacacaaactctagccccgacgaccagatcggctactatagacgggccaccagaaggatcagaggaggtgatggcaagatgaaggacctgagccctagatggtacttctactacctgggcacaggcccagaagccggcctgccttacggcgccaacaaggacggcatcatctgggtcgccaccgagggcgctctcaacacccctaaggaccacattggaactcggaaccccgctaataacgccgctatcgtgctgcagctgcctcagggcacgaccctgcccaagggcttctacgccgaaggcagcagaggcggcagccaggcctctagccggtccagctctcggagcagaaacagcagcagaaactccacccctggcagcagccgcggcaccagccccgccagaatggccggaaatggcggcgatgccgctctggccctgctgctgctggatagactgaaccagctggaatccaagatgtctggcaagggccagcagcaacagggccagaccgtgaccaagaaaagcgcagctgaagcctctaaaaaacctcggcagaagcggaccgccacaaaggcttacaacgtgacacaggcctttggcagaagaggacctgagcagacacagggcaacttcggcgaccaggagctgatccggcagggcacagactacaagcattggcctcagatcgcccagttcgcccctagtgccagcgccttcttcggcatgagccggatcggcatggaagtgacccctagcggcacatggctgacctacaccggcgccatcaagctggacgataaggaccccaattttaaggaccaagtgatcctgctgaacaagcacatcgacgcctataagaccttcccacctacagagcctaagaaagataagaaaaagaaggccgacgagacacaagccctgccccagagacagaaaaagcaacaaacagtgaccctgctgcctgccgctgatctggatgacttcagcaagcagctgcagcaatctatgagctccgccgatagcacccaggcctga

SEQ ID NO: 8 - DNA, 3822 bp, Artificial Sequence
  Nucleotide sequence of SARS-CoV-2 spike (S) protein
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccaggacgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcgcagagccagaagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 9 - DNA, 3822 bp, Artificial Sequence
  Nucleotide sequence of SARS-CoV-2 modified spike (S) protein
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccaggacgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 10 - DNA, 13283 bp, Artificial Sequence
  Nucleotide sequence of construct F548
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 11 - DNA, 13283 bp, Artificial Sequence
  Nucleotide sequence of construct F549
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagctctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 12 - DNA, 13809 bp, Artificial Sequence
  Nucleotide sequence of construct F556
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 13 - DNA, 13809 bp, Artificial Sequence
  Nucleotide sequence of construct F557
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 14 - DNA, 13295 bp, Artificial Sequence
  Nucleotide sequence of construct F602
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaagggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 15 - DNA, 101 bp, Artificial Sequence
  Nucleotide sequence of extended subgenomic promoter (v4)
actctagctagcagtgttaaatcattcagctacctgagaggggcccctataactctctacggctaacctgaatggactacgacatagtctagtccgccaag

SEQ ID NO: 16 - DNA, 759 bp, Artificial Sequence
  Nucleotide sequence of influenza A virus M1 matrix protein (PR8X)
atgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtga

SEQ ID NO: 17 - DNA, 294 bp, Artificial Sequence
  Nucleotide sequence of influenza A virus M2 matrix protein
atgagtcttctaaccgaggtcgaaacgcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 18 - DNA, 660 bp, Artificial Sequence
  Nucleotide sequence of influenza A virus NS1 non-structural protein (A/California/09)
atggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcgctacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatga

SEQ ID NO: 19 - DNA, 14091 bp, Artificial Sequence
  Nucleotide sequence of construct F554
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagctctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 20 - DNA, 12593 bp, Artificial Sequence
  Nucleotide sequence of construct F568
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaactctctacggctaacctgaatggactacgacatagtctagtccgccaagatggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcgctacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 21 - DNA, 12593 bp, Artificial Sequence
  Nucleotide sequence of construct F569
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcgctacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatgactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 22 - DNA, 13032 bp, Artificial Sequence
  Nucleotide sequence of construct F570
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgagcaagatctacatcgacgagcggagcaacgccgagattgtgtgcgaggccatcaagaccatcggaatcgaaggcgccacagccgctcagctgaccagacagctgaacatggaaaagcgggaagtgaacaaggccctgtacgacctgcagagaagcgccatggtgtacagcagcgacgacatccctcctcggtggtttatgaccacagaggccgacaagcctgacgccgatgctatggccgacgtgatcatcgacgacgtgtcccgcgagaagtccatgagagaggaccacaagagcttcgacgatgtgatccccgccaagaagatcatcgattggaagggcgccaatcctgtgaccgtgatcaacgagtactgccagatcaccagaagagactggtccttccggatcgagagcgtgggacctagcaatagccctaccttctacgcctgcgtggacatcgacggcagagtgttcgataaggccgacggcaagagcaagcgggacgccaaaaacaatgccgccaagctggccgtggataagctgctgggctatgtgatcatccggttctaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 23 - DNA, 12692 bp, Artificial Sequence
  Nucleotide sequence of construct F576
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaactctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 24 - DNA, 14091 bp, Artificial Sequence
  Nucleotide sequence of construct F584
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagctctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 25 - DNA, 14434 bp, Artificial Sequence
  Nucleotide sequence of construct F590
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgactctctacggctaacctgaatggactacgacatagtctagtccgccaagatgagtcttctaaccgaggtcgaaacgcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagctctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 26 - DNA, 13314 bp, Artificial Sequence
  Nucleotide sequence of construct F616
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaatcattcagctacctgagaggggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtagggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 27 - DNA, 12509 bp, Artificial Sequence
  Nucleotide sequence of construct F620
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtgagggcccctataactctctacggctaacctgaatggactacgacatagtctagtccgccaagatggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaggagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactgagaagcagatactgggccataaggaccaggagtggaggaaataccaatcaacaaaaggcatccgcaggccagatcagtgtgcagcctacattctcagtgcagcggaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagttgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 28 - DNA, 1497 bp, Artificial Sequence
  Nucleotide sequence of influenza virus nucleoprotein (A/California/09)
atggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaggagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactgagaagcagatactgggccataaggaccaggagtggaggaaataccaatcaacaaaaggcatccgcaggccagatcagtgtgcagcctacattctcagtgcagcggaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagttga

SEQ ID NO: 29 - DNA, 759 bp, Artificial Sequence
  Nucleotide sequence of influenza A virus M1 matrix protein (A/California/09)
atgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtga

SEQ ID NO: 30 - DNA, 15335 bp, Artificial Sequence
  Nucleotide sequence of construct Co18
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccaggacgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctgagggcccctataactctctacggctaacctgaatggactacgacatagtctagtccgccaagatgagcgacaacggacctcagaaccagagaaatgcccctagaatcacctttggcggacctagcgacagcaccggcagcaaccagaatggcgagagaagcggcgccagatctaagcagcggcgtccacagggactgcccaacaacaccgccagctggttcaccgccctcacccagcacggcaaagaggacctgaagttcccccggggacagggcgtgccaatcaacacaaactctagccccgacgaccagatcggctactatagacgggccaccagaaggatcagaggaggtgatggcaagatgaaggacctgagccctagatggtacttctactacctgggcacaggcccagaagccggcctgccttacggcgccaacaaggacggcatcatctgggtcgccaccgagggcgctctcaacacccctaaggaccacattggaactcggaaccccgctaataacgccgctatcgtgctgcagctgcctcagggcacgaccctgcccaagggcttctacgccgaaggcagcagaggcggcagccaggcctctagccggtccagctctcggagcagaaacagcagcagaaactccacccctggcagcagccgcggcaccagccccgccagaatggccggaaatggcggcgatgccgctctggccctgctgctgctggatagactgaaccagctggaatccaagatgtctggcaagggccagcagcaacagggccagaccgtgaccaagaaaagcgcagctgaagcctctaaaaaacctcggcagaagcggaccgccacaaaggcttacaacgtgacacaggcctttggcagaagaggacctgagcagacacagggcaacttcggcgaccaggagctgatccggcagggcacagactacaagcattggcctcagatcgcccagttcgcccctagtgccagcgccttcttcggcatgagccggatcggcatggaagtgacccctagcggcacatggctgacctacaccggcgccatcaagctggacgataaggaccccaattttaaggaccaagtgatcctgctgaacaagcacatcgacgcctataagaccttcccacctacagagcctaagaaagataagaaaaagaaggccgacgagacacaagccctgccccagagacagaaaaagcaacaaacagtgaccctgctgcctgccgctgatctggatgacttcagcaagcagctgcagcaatctatgagctccgccgatagcacccaggcctgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 31 - DNA, 15354 bp, Artificial Sequence
  Nucleotide sequence of construct Co19
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccaggacgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctgatcattcagctacctgagaggggcccctataactctctacggctaacctgaatggactacgacatagtctagtccgccaagatgagcgacaacggacctcagaaccagagaaatgcccctagaatcacctttggcggacctagcgacagcaccggcagcaaccagaatggcgagagaagcggcgccagatctaagcagcggcgtccacagggactgcccaacaacaccgccagctggttcaccgccctcacccagcacggcaaagaggacctgaagttcccccggggacagggcgtgccaatcaacacaaactctagccccgacgaccagatcggctactatagacgggccaccagaaggatcagaggaggtgatggcaagatgaaggacctgagccctagatggtacttctactacctgggcacaggcccagaagccggcctgccttacggcgccaacaaggacggcatcatctgggtcgccaccgagggcgctctcaacacccctaaggaccacattggaactcggaaccccgctaataacgccgctatcgtgctgcagctgcctcagggcacgaccctgcccaagggcttctacgccgaaggcagcagaggcggcagccaggcctctagccggtccagctctcggagcagaaacagcagcagaaactccacccctggcagcagccgcggcaccagccccgccagaatggccggaaatggcggcgatgccgctctggccctgctgctgctggatagactgaaccagctggaatccaagatgtctggcaagggccagcagcaacagggccagaccgtgaccaagaaaagcgcagctgaagcctctaaaaaacctcggcagaagcggaccgccacaaaggcttacaacgtgacacaggcctttggcagaagaggacctgagcagacacagggcaacttcggcgaccaggagctgatccggcagggcacagactacaagcattggcctcagatcgcccagttcgcccctagtgccagcgccttcttcggcatgagccggatcggcatggaagtgacccctagcggcacatggctgacctacaccggcgccatcaagctggacgataaggaccccaattttaaggaccaagtgatcctgctgaacaagcacatcgacgcctataagaccttcccacctacagagcctaagaaagataagaaaaagaaggccgacgagacacaagccctgccccagagacagaaaaagcaacaaacagtgaccctgctgcctgccgctgatctggatgacttcagcaagcagctgcagcaatctatgagctccgccgatagcacccaggcctgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 32 - DNA, 3822 bp, Artificial Sequence
  Nucleotide sequence of SARS-CoV-2 spike (S) protein uncleavable (S1/S2 RRAR to QQAA mutation and 986P/987P mutation)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccaggacgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacccacctgaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 33 - DNA, 3822 bp, Artificial Sequence
  Nucleotide sequence of SARS-CoV-2 modified spike (S) protein uncleavable (S1/S2 RRAR to QQAA mutation and D614G mutation)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 34 - DNA, 3822 bp, Artificial Sequence
  Nucleotide sequence of SARS-CoV-2 modified spike (S) protein uncleavable (S1/S2 RRAR to QQAA mutation and S2′ mutation)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccaggacgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagaacagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 35 - DNA, 3822 bp, Artificial Sequence
  Nucleotide sequence of SARS-CoV-2 modified spike (S) protein uncleavable (S1/S2 RRAR to QQAA mutation and D614G mutation and S2′ mutation)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagaacagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 36 - DNA, 3822 bp, Artificial Sequence
  Nucleotide sequence of SARS-CoV-2 modified spike (S) protein cleavable (D614G mutation)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacaaatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcgcagagccagaagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 37 - protein, 1273 aa, Artificial Sequence
  Amino acid sequence of SARS-CoV-2 S protein full length wt
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - DNA, 7 bp, Artificial Sequence
  Kozak consensus sequence
accatgg

SEQ ID NO: 39 - DNA, 6 bp, Artificial Sequence
  Kozak consensus sequence
accatg

SEQ ID NO: 40 - RNA, 10 nt, Artificial Sequence
  Interrupting linker
gcauaugacu

SEQ ID NO: 41 - DNA, 10 bp, Artificial Sequence
  GC-rich element
ccccggcgcc

SEQ ID NO: 42 - DNA, 7 bp, Artificial Sequence
  GC-rich element
ccccggc

SEQ ID NO: 43 - DNA, 20 bp, Artificial Sequence
  GC-rich element
gcgccccgcggcgccccgcg

SEQ ID NO: 44 - DNA, 24 bp, Artificial Sequence
  Histone stem loop
caaaggctcttttcagagccacca

SEQ ID NO: 45 - DNA, 45 bp, Venezuelan equine encephalitis virus
gataggcggcgcatgagagaagcccagaccaattacctacccaaa

SEQ ID NO: 46 - DNA, 341 bp, Sindbis virus
ggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 47 - DNA, 71 bp, Artificial Sequence
  Nucleotide sequence of extended subgenomic promoter
gggcccctataactctctacggctaacctgaatggacctgaatggactacgacatagtctagtccgccaag

SEQ ID NO: 48 - DNA, 36 bp, Artificial Sequence
  poly-a sequence
aaaaaaaaaaaaaaaaaaaaaaaaaaaaaaaaaaaa

SEQ ID NO: 49 - DNA, 13409 bp, Artificial Sequence
  Nucleotide sequence of construct 631
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaaggcaataattgtactactcatggtagtaacatccaatgcagatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggatttttttcaacaatggcttgggctgttccaaaggacaactacaaaaatgcaacgaacccacaaacagtggaagtaccatacatttgtacagaaggggaagaccaaattactgtttgggggttccattcggataacaaaacccaaatgaagtccctctatggagactcaaatcctcaaaagttcacctcatctgctaatggagtgaccacacattatgtttctcaaattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaatattctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatttgtctataagggcccctataactctctacggctaacctgaatggactacgacatagtctagtccgccaagatgctaccttcaactatacaaacgttaaccttatttctcacattagggggagtgctattatcactatatgtgtcagcttcattgtcatacttactatattcggatatattgctaaaattttcacgaacagaagtaactgcaccaataatgccattggattgtgcaaacgcatcaaatgtccaggctgtgaatcgttctgcaacaaaaggggtgacacctcttctcccagaaccggagtggacgtaccctcgtttatcttgcccgggctcaacctttcagaaagcactcctaattagcccccatagattcggagaaaccaaaggaaactcagctcccttaataataagggaaccttttattgcttgtggaccaaaggaatgcaaacattttgctctaacccattatgcagctcaaccagggggatactacaatggaacaagagaagacagaaacaagctgaggcatctaatttcagtcaaattgggcaaaatcccaacagtagaaaactctattttccacatggcagcttggagtgggtccgcatgccatgatggtagagaatggacttacatcggagttgatggcccagacagtaatgcattgctcaaaataaaatatggagaagcatatactgacacataccattcctatgcaaaaaacatcctaaggacacaagaaagtgcctgcaattgcatcgggggagattgttatctgatgataactgatggcccagcttcagggattagtgaatgcagattccttaagattcgagagggccgaataataaaagaaatatttccaacaggaagagtaaaacacactgaggaatgcacatgcggatttgccagcaacaaaaccatagaatgtgcctgtagagataacagttacacagcaaaaagaccctttgtcaaattaaatgtggagaccgatacagcggaaataagattgatgtgcacaaagacttatttggacacccccagaccaaatgatggaagcataacagggccttgcgaatctgatggaaacgaagggggtggaggcatcaagggaggatttgttcaccaaagaatggcatccaagattggaaggtggtactctcggacgatgtctaaaactcaaagaatggggatgggactgtatgtaaagtatgatggagacccatggactgacagtgaagcccttgctcttagtggagtaatggtttcaatgggagaacctggttggtattcctttggcttcgaaataaaagataagaaatgtgatgtcccctgtattgggatagaaatggtacatgatggtgggaaaacgacttggcactcagcggcaacagccatttactgtttaatgggctcaggacaactgctgtgggacactgtcacaggtgttaatatgactctgtaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 50 - DNA, 13295 bp, Artificial Sequence
  Nucleotide sequence of construct 632
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaatccaaatcagaagataataaccatcggatcaatctgtatggtaattggaatagttagcttaatgttacaaattgggaacatgatctcaatatgggtcagtcattcaattcagacagggaatcaatgccaagctgaaccaatcagcaatactaaatttcttactgagaaagctgtggcttcagtaacattagcgggcaattcatctctttgccccattagcggatgggctgtatacagtaaggacaacagtataaggatcggttccaggggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggtccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtagtgggcaggcatcatataagatcttcaaaatggaaaaagggaaagtggttaaatcagtcgaattggatgctcctaattatcactatgaggagtgctcctgttatcctgatgccggcgaaatcacatgtgtgtgcagggataattggcatggctcaaataggccatgggtatctttcaatcaaaatttggagtatcaaataggatatatatgcagtggagttttcggagacaatccacgccccaatgatggaacaggtagttgtggtccggtgttctctaacggggcatatggggtaaaagggttttcattcaaatacggcaatggtgtttggatcgggagaaccaaaagcactaattccaggagcggctttgaaatgatttgggacccaaatgggtggactggaacggacagtagcttttcggtgaagcaagatatcgtagcaataactgattggtcaggatatagcgggagttttgtccagcatccagaactgacaggattagattgcataagaccttgtttctgggttgagttaatcagagggcggcctaaagagagcacaatttggaccagtgggagcagcatatctttttgtggtgtaaatagtgacactgttagttggtcttggccagacggtgctgagttgccattcaccattgacaagtaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 51 - DNA, 13364 bp, Artificial Sequence
  Nucleotide sequence of construct 629
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaagactatcattgctttgagctacattctatgtctggttttcactcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtaaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagtttttcttgcataaggggatctagtagtagtttctttagtagattaaattggttgacccatttaaactacacatatccagcactgaacgtgactatgccaaacaaggaacaatttgacaaattgtacatttggggggttcaccacccgggtacggacaaggaccaaatctccctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtgaacaggatcacatacggggcctgtcccagatatgttaagcaaagcactctgaaattggcaacaggaatgcgaaatgtaccagagaaacaaactagaggcatatttggtgcaatagcgggtttcatagaaaatggttgggagggaatggtggatggttggtacggtttcagacatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaacgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatctgcatttgagggcccctataactctctacggctaacctgaatggactacgacatagtctagtccgccaagatgaatccaaatcaaaagataataacgattggctctgtttctctcacaatttccacaatatgcttcttcatgcaaattgccatcctgataactactgtaacattgcatttcaagcaatatgaattcaactcccccccgaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtgacattacaggatttgcacctttctctaaggacaattcgattaggctttccgctggtggggacatctgggtgacaagagaaccttatgtgtcatgcgaccttgacaagtgttatcaatttgcacttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgccacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcagggcttgttggagacacacccagaaaaagcgacagctccagcagtagccattgtttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagttgacagaggtgataggtccggttattctggtattttctctgttgaaggcaaaagctgcatcaatcggtgcttctatgtggagttgattaggggaagaaaagaggaaactgaagtcttgtggacctcaaacagtattgttgtgttttgtggcacctcaggtacatatggaacaggctcatggcctgatggggcggacctcaatctcatgcatacataaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 52 - DNA, 13409 bp, Artificial Sequence
  Nucleotide sequence of construct 695
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgctaccttcaactatacaaacgttaaccttatttctcacattagggggagtgctattatcactatatgtgtcagcttcattgtcatacttactatattcggatatattgctaaaattttcacgaacagaagtaactgcaccaataatgccattggattgtgcaaacgcatcaaatgtccaggctgtgaatcgttctgcaacaaaaggggtgacacctcttctcccagaaccggagtggacgtaccctcgtttatcttgcccgggctcaacctttcagaaagcactcctaattagcccccatagattcggagaaaccaaaggaaactcagctcccttaataataagggaaccttttattgcttgtggaccaaaggaatgcaaacattttgctctaacccattatgcagctcaaccagggggatactacaatggaacaagagaagacagaaacaagctgaggcatctaatttcagtcaaattgggcaaaatcccaacagtagaaaactctattttccacatggcagcttggagtgggtccgcatgccatgatggtagagaatggacttacatcggagttgatggcccagacagtaatgcattgctcaaaataaaatatggagaagcatatactgacacataccattcctatgcaaaaaacatcctaaggacacaagaaagtgcctgcaattgcatcgggggagattgttatctgatgataactgatggcccagcttcagggattagtgaatgcagattccttaagattcgagagggccgaataataaaagaaatatttccaacaggaagagtaaaacacactgaggaatgcacatgcggatttgccagcaacaaaaccatagaatgtgcctgtagagataacagttacacagcaaaaagaccctttgtcaaattaaatgtggagaccgatacagcggaaataagattgatgtgcacaaagacttatttggacacccccagaccaaatgatggaagcataacagggccttgcgaatctgatggaaacgaagggggtggaggcatcaagggaggatttgttcaccaaagaatggcatccaagattggaaggtggtactctcggacgatgtctaaaactcaaagaatggggatgggactgtatgtaaagtatgatggagacccatggactgacagtgaagcccttgctcttagtggagtaatggtttcaatgggagaacctggttggtattcctttggcttcgaaataaaagataagaaatgtgatgtcccctgtattgggatagaaatggtacatgatggtgggaaaacgacttggcactcagcggcaacagccatttactgtttaatgggctcaggacaactgctgtgggacactgtcacaggtgttaatatgactctgtaagggcccctataactctctacggctaacctgaatggactacgacatagtctagtccgccaagatgaaggcaataattgtactactcatggtagtaacatccaatgcagatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggatttttttcaacaatggcttgggctgttccaaaggacaactacaaaaatgcaacgaacccacaaacagtggaagtaccatacatttgtacagaaggggaagaccaaattactgtttgggggttccattcggataacaaaacccaaatgaagtccctctatggagactcaaatcctcaaaagttcacctcatctgctaatggagtgaccacacattatgtttctcaaattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaatattctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatttgtctataaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 53 - DNA, 13364 bp, Artificial Sequence
  Nucleotide sequence of construct 703
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaatccaaatcaaaagataataacgattggctctgtttctctcacaatttccacaatatgcttcttcatgcaaattgccatcctgataactactgtaacattgcatttcaagcaatatgaattcaactcccccccgaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtgacattacaggatttgcacctttctctaaggacaattcgattaggctttccgctggtggggacatctgggtgacaagagaaccttatgtgtcatgcgaccttgacaagtgttatcaatttgcacttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgccacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcagggcttgttggagacacacccagaaaaagcgacagctccagcagtagccattgtttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagtggatagaggtgataggtccggatacagcggtattttctctgttgaaggcaaaagctgcatcaatcggtgcttctatgtggagttgattaggggaagaaaagaggaaactgaagtcttgtggacctcaaacagtattgttgtgttttgtggcacctcaggtacatatggaacaggctcatggcctgatggggcggacctcaatctcatgcatacataagggcccctataactctctacggctaacctgaatggactacgacatagtctagtccgccaagatgaagactatcattgctttgagctacattctatgtctggttttcactcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtaaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagtttttcttgcataaggggatctagtagtagtttctttagtagattaaattggttgacccatttaaactacacatatccagcactgaacgtgactatgccaaacaaggaacaatttgacaaattgtacatttggggggttcaccacccgggtacggacaaggaccaaatctccctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtgaacaggatcacatacggggcctgtcccagatatgttaagcaaagcactctgaaattggcaacaggaatgcgaaatgtaccagagaaacaaactagaggcatatttggtgcaatagcgggtttcatagaaaatggttgggagggaatggtggatggttggtacggtttcagacatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaacgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatctgcatttgaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 54 - DNA, 1701 bp, Artificial Sequence
  Nucleotide sequence of influenza A virus H3 protein (A/Delaware/39/2019)
atgaagactatcattgctttgagctacattctatgtctggttttcactcaaaaaattcctggaaatgacaatagcacggcaacgctgtgccttgggcaccatgcagtaccaaacggaacgatagtgaaaacaatcacaaatgaccgaattgaagttactaatgctactgagttggttcagaattcctcaataggtaaaatatgcgacagtcctcatcagatccttgatggagggaactgcacactaatagatgctctattgggggaccctcagtgtgacggctttcaaaataagaaatgggacctttttgttgaacgaagcagagcctacagcaactgttacccttatgatgtgccggattatgcctcccttaggtcactagttgcctcatccggcacactggagtttaaaaatgaaagcttcaattgggctggagtcactcaaaacggaaaaagtttttcttgcataaggggatctagtagtagtttctttagtagattaaattggttgacccatttaaactacacatatccagcactgaacgtgactatgccaaacaaggaacaatttgacaaattgtacatttggggggttcaccacccgggtacggacaaggaccaaatctccctgtatgctcaatcatcaggaagaatcacagtatctaccaaaagaagccaacaagctgtaatcccaaatatcggatctagacccagaataagggatatccctagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaagtctgaatgcatcactccaaatggaagcattcccaatgacaaaccattccaaaatgtgaacaggatcacatacggggcctgtcccagatatgttaagcaaagcactctgaaattggcaacaggaatgcgaaatgtaccagagaaacaaactagaggcatatttggtgcaatagcgggtttcatagaaaatggttgggagggaatggtggatggttggtacggtttcagacatcaaaattctgagggaagaggacaagcagcagatctcaaaagcactcaagcagcaatcgatcaaatcaatgggaagctgaatcgattgatcggaaaaaccaacgagaaattccatcagattgaaaaagaattctcagaagtagaaggaagagttcaagaccttgagaaatatgttgaggacactaaaatagatctctggtcatacaacgcggagcttcttgttgccctggagaaccaacatacaattgatctaactgactcagaaatgaacaaactgtttgaaaaaacaaagaagcaactgagggaaaatgctgaggatatgggaaatggttgtttcaaaatataccacaaatgtgacaatgcctgcataggatcaataagaaacgaaacttatgaccacaatgtgtacagggatgaagcattaaacaaccggttccagatcaagggagttgagctgaagtcagggtacaaagattggatcctatggatttcctttgccatatcatgttttttgctttgtgttgctttgttggggttcatcatgtgggcctgccaaaagggcaacattagatgcaacatctgcatttga

SEQ ID NO: 55 - DNA, 1410 bp, Artificial Sequence
  Nucleotide sequence of influenza A virus N2 protein (A/Delaware/39/2019)
atgaatccaaatcaaaagataataacgattggctctgtttctctcacaatttccacaatatgcttcttcatgcaaattgccatcctgataactactgtaacattgcatttcaagcaatatgaattcaactcccccccgaataaccaagtgatgctgtgtgaaccaacaataatagaaagaaacataacagagatagtgtatttgaccaacaccaccatagagaaggaaatatgccccaaaccagcagaatacagaaattggtcaaaaccgcaatgtgacattacaggatttgcacctttctctaaggacaattcgattaggctttccgctggtggggacatctgggtgacaagagaaccttatgtgtcatgcgaccttgacaagtgttatcaatttgcacttggacagggaacaacactaaacaacgtgcattcaaataacacagtacgtgataggaccccttatcggactctattgatgaatgagttgggtgttcctttccatctggggaccaagcaagtgtgcatagcatggtccagctcaagttgtcacgatggaaaagcatggctgcatgtttgtataacgggggatgataaaaatgcaactgctagcttcatttacaatgggaggcttgtagatagtgttgtttcatggtccaacgatattctcaggacccaggagtcagaatgcgtttgtatcaatggaacttgtacagtagtaatgactgatggaaatgccacaggaaaagctgatactaaaatactattcattgaggaggggaaaatcgttcatactagcaaattgtcaggaagtgctcagcatgtcgaagagtgctcttgctatcctcgatatcctggtgtcagatgtgtctgcagagacaactggaaaggatccaaccggcccatcatagatataaacataaaggatcatagcattgtttccagttatgtgtgttcagggcttgttggagacacacccagaaaaagcgacagctccagcagtagccattgtttgaatcctaacaatgaaaaaggtggtcatggagtgaaaggctgggcctttgatgatggaaatgacgtgtggatggggagaacaatcaacgagacgtcacgcttagggtatgaaaccttcaaagtcgttgaaggctggtccaaccctaagtccaaattgcagataaataggcaagtcatagtggatagaggtgataggtccggatacagcggtattttctctgttgaaggcaaaagctgcatcaatcggtgcttctatgtggagttgattaggggaagaaaagaggaaactgaagtcttgtggacctcaaacagtattgttgtgttttgtggcacctcaggtacatatggaacaggctcatggcctgatggggcggacctcaatctcatgcatacataa

SEQ ID NO: 56 - DNA, 1755 bp, Artificial Sequence
  Nucleotide sequence of influenza B virus Hyam (B/Singapore/INFTT 16 0610/16 (By))
atgaaggcaataattgtactactcatggtagtaacatccaatgcagatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggatttttttcaacaatggcttgggctgttccaaaggacaactacaaaaatgcaacgaacccacaaacagtggaagtaccatacatttgtacagaaggggaagaccaaattactgtttgggggttccattcggataacaaaacccaaatgaagtccctctatggagactcaaatcctcaaaagttcacctcatctgctaatggagtgaccacacattatgtttctcaaattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaatattctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatttgtctataa

SEQ ID NO: 57 - DNA, 1401 bp, Artificial Sequence
  Nucleotide sequence of influenza B virus Nyam (B/Singapore/INFTT 16 0610/16 (By))
atgctaccttcaactatacaaacgttaaccttatttctcacattagggggagtgctattatcactatatgtgtcagcttcattgtcatacttactatattcggatatattgctaaaattttcacgaacagaagtaactgcaccaataatgccattggattgtgcaaacgcatcaaatgtccaggctgtgaatcgttctgcaacaaaaggggtgacacctcttctcccagaaccggagtggacgtaccctcgtttatcttgcccgggctcaacctttcagaaagcactcctaattagcccccatagattcggagaaaccaaaggaaactcagctcccttaataataagggaaccttttattgcttgtggaccaaaggaatgcaaacattttgctctaacccattatgcagctcaaccagggggatactacaatggaacaagagaagacagaaacaagctgaggcatctaatttcagtcaaattgggcaaaatcccaacagtagaaaactctattttccacatggcagcttggagtgggtccgcatgccatgatggtagagaatggacttacatcggagttgatggcccagacagtaatgcattgctcaaaataaaatatggagaagcatatactgacacataccattcctatgcaaaaaacatcctaaggacacaagaaagtgcctgcaattgcatcgggggagattgttatctgatgataactgatggcccagcttcagggattagtgaatgcagattccttaagattcgagagggccgaataataaaagaaatatttccaacaggaagagtaaaacacactgaggaatgcacatgcggatttgccagcaacaaaaccatagaatgtgcctgtagagataacagttacacagcaaaaagaccctttgtcaaattaaatgtggagaccgatacagcggaaataagattgatgtgcacaaagacttatttggacacccccagaccaaatgatggaagcataacagggccttgcgaatctgatggaaacgaagggggtggaggcatcaagggaggatttgttcaccaaagaatggcatccaagattggaaggtggtactctcggacgatgtctaaaactcaaagaatggggatgggactgtatgtaaagtatgatggagacccatggactgacagtgaagcccttgctcttagtggagtaatggtttcaatgggagaacctggttggtattcctttggcttcgaaataaaagataagaaatgtgatgtcccctgtattgggatagaaatggtacatgatggtgggaaaacgacttggcactcagcggcaacagccatttactgtttaatgggctcaggacaactgctgtgggacactgtcacaggtgttaatatgactctgtaa

SEQ ID NO: 58 - DNA, 3813 bp, Artificial Sequence
  Nucleotide sequence of SARS-CoV-2 spike (S) protein (RRAR?QQAA; ?69-70; ?Y144; N501Y; D614G)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatctctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacatatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 59 - DNA, 3813 bp, Artificial Sequence
  Nucleotide sequence of SARS-CoV-2 spike (S) protein (RRAR to QQAA; Del242-244; K417N; E484K; N501Y; D614G)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaacatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtgaaaggctttaactgctacttccctctgcaaagctatggatttcaacctacatatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 60 - DNA, 3804 bp, Artificial Sequence
  Nucleotide sequence of SARS-CoV-2 spike (S) protein (RRAR to QQAA; del69-70; del242-244; K417N; E484K; N501Y; D614G)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatctctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaacatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtgaaaggctttaactgctacttccctctgcaaagctatggatttcaacctacatatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagccgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 61 - DNA, 3813 bp, Artificial Sequence
  Nucleotide sequence of SARS-CoV-2 spike (S) protein (RRAR toQQAA; del69-70; delY144; N501Y; A570D; D614G; P680H; T716I)
atgttcgtgttcctggtgctgctgcccctcgttagcagccagtgcgtgaatctgaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatctctggcaccaacggcacaaagcgcttcgacaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcgggacctgccacaaggcttcagcgccctggaacctctggtggacctgcctatcggcatcaacatcacacggttccaaaccctgctggccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaagatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtggaaggctttaactgctacttccctctgcaaagctatggatttcaacctacatatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgacgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccatcagcaagccgctagcgtggcctcccagagcatcatcgcctacaccatgagcctgggagccgagaactctgtggcctacagcaacaacagcatcgctatccctatcaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctggctagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacacataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

SEQ ID NO: 62 - DNA, 3813 bp, Artificial Sequence
  Nucleotide sequence of SARS-CoV-2 spike (S) protein (RRAR toQQAA; L18F; D80A; D215G; ?242-244; K417N; E484K; N501Y; D614G; A701V)
atgttcgtgttcctggtgctgctgcccctcgttagcagccagtgcgtgaatttcaccacccgcacccagctgccaccagcctacacaaacagcttcaccagaggagtgtattaccctgataaggtctttagatcctccgtcctgcattctacgcaggatctcttcttgccattcttcagcaacgtgacatggttccacgccatccacgtttctggcaccaacggcacaaagcgcttcgccaatcctgtgttgccgtttaacgacggcgtttacttcgccagcacagaaaagagcaacatcatccggggctggatcttcggcaccaccctggacagcaaaacccaaagcctgctcatcgtgaacaacgccaccaacgtggtgatcaaggtgtgcgagttccagttctgcaatgatccttttctgggcgtgtactatcacaagaacaacaagagctggatggaaagcgagttcagagtgtattctagcgccaacaactgcacctttgagtacgtgtcccagccctttcttatggacctggaaggcaagcagggcaacttcaagaatctgagagaattcgtgttcaagaacattgatggctacttcaagatctacagcaagcacacccctatcaacctggttcggggcctgccacaaggcttcagcgccctggaacctctggtggacctgcctatcggcatcaacatcacacggttccaaaccctgcaccggagctacctgacccccggcgacagcagcagcggctggaccgccggcgctgccgcctattacgtgggctacctgcaacctagaaccttcctgctgaaatacaacgagaacggcacaatcaccgacgccgtggactgtgccctggaccccctgtctgagacaaagtgtaccctgaagtctttcaccgtggagaagggcatctaccagaccagcaacttccgggtgcagcctacagaatctatagtgcggttccctaacatcaccaacctgtgtccttttggcgaggtgttcaacgccactcggttcgcctctgtctacgcctggaaccggaaacggatctctaattgcgtggccgattacagcgtcctgtataactccgccagtttcagcacattcaagtgctacggcgtgtcacccaccaagctgaacgatctgtgcttcaccaatgtgtacgccgatagtttcgtgatccggggcgatgaggtgcggcagatcgcccctggacagacaggcaatatcgccgactacaactacaagctgcctgacgacttcacaggctgtgtgatcgcatggaacagcaacaacctggacagcaaggtgggcggaaactacaactacctgtacagactgttcagaaagtccaacctgaagcctttcgagagagatatatctaccgagatctaccaggccggcagcacaccctgtaatggagtgaaaggctttaactgctacttccctctgcaaagctatggatttcaacctacatatggggttggctaccagccttacagagtggtggtccttagcttcgagctgctccatgcccctgccaccgtgtgcggacctaagaagtccaccaacctggtgaaaaacaagtgcgtgaactttaattttaacggcctgaccggaacaggagtgctgacagaaagcaacaaaaagttcctgcctttccagcagttcggcagagacattgccgacaccacagatgctgttagagacccccagacgctggaaatcctggatatcaccccctgctcttttggcggcgtgagcgtgatcaccccaggcacaaacacaagcaaccaggtggctgtgctgtaccagggcgtgaactgtacagaggtccctgtggcaatccacgccgatcagctgacccctacatggcgggtgtactccactggatctaacgtgttccagacaagggccggatgcctcatcggcgctgagcacgtgaacaattcttacgagtgcgacatccctattggagcgggcatctgcgccagctaccagacacagaccaatagccctcagcaagccgctagcgtggcctcccagagcatcatcgcctacaccatgagcctgggagtcgagaactctgtggcctacagcaacaacagcatcgctatccctaccaacttcaccatctctgtcaccaccgaaatcctgcccgtcagtatgaccaaaaccagcgtcgactgcaccatgtacatatgcggcgatagcaccgaatgcagcaacctgctgctgcagtatggctccttctgcacccaacttaacagagccctgactggcatcgccgtggagcaggacaagaatacccaggaggtgttcgcccaggtgaagcagatctacaagacacccccgatcaaggacttcggcggctttaatttctctcagatcctgccagacccatctaaaccctctaagcggagctttatcgaggacctgctgttcaacaaggtgactctggctgacgccggcttcatcaagcagtacggcgattgcctgggcgacattgctgctagagacctgatctgtgcccagaaattcaacggtcttactgtgctgcctcctctgctgacggatgagatgatcgcccagtacaccagcgccctgctggccggcaccatcacatccggctggacattcggcgccggcgcagccctgcagatcccttttgccatgcagatggcctaccggttcaacggaatcggagtgacacagaacgtgctctacgaaaatcagaagttgatcgccaaccagttcaacagcgccatcggcaagattcaggatagtctgagttccaccgccagcgccctgggaaagctgcaggacgtggtcaatcagaatgcccaagccctgaacaccctggtgaagcagctgagcagcaacttcggcgccatcagctctgtgctgaacgacatcctgagtagactggacaaggtggaagccgaagtgcagatcgacagattgatcaccggaagactgcaaagcctgcagacctacgtgacccagcagctgataagagctgctgaaatcagagccagcgctaatctggccgctaccaagatgagcgagtgcgttctgggccagtctaagagagtggacttctgcggaaaaggctaccacctgatgtcctttcctcagtctgccccccacggcgtggtgttcctgcacgtcacatacgtgcccgctcaagagaaaaacttcaccacggcccctgccatctgtcacgacggcaaggcccacttccccagagagggcgtgttcgtgagcaatggcacccactggtttgtgactcagagaaacttctacgagccacagattatcaccacagataacaccttcgtgtctggcaactgcgacgtggtgatcggcatcgtcaacaacacagtgtacgacccactgcaacctgagctggactcattcaaggaggaactggataagtacttcaagaatcacaccagccccgacgttgacctgggcgacatcagcggcattaacgcctctgtggtcaacatccagaaggaaatcgacagactgaatgaggtggccaagaatttgaacgagagcctgattgatctgcaggagctgggcaaatacgagcagtacatcaagtggccttggtacatctggctgggcttcatcgccgggctgatcgccatcgttatggtgacaatcatgctgtgttgcatgacaagctgttgtagctgcctgaaaggctgctgctcctgcggcagctgttgcaagtttgacgaagatgacagcgagcccgtgctgaaaggcgtcaagctgcactacacctga

(19) United States
(12) Patent Application Publication
(10) Pub. No.: US 2024/0024455 A1
(43) Pub. Date: Jan. 25, 2024

(54) MULTICISTRONIC RNA VACCINES AND USES THEREOF

(71) Applicant: SEQIRUS INC., Holly Springs, NC (US)

(72) Inventors: Pirada ALLEN, Holly Springs, NC (US); Ivna DE SOUZA, Holly Springs, NC (US); Yingxia WEN, Holly Springs, NC (US); Sukhmani BEDI, Holly Springs, NC (US); Cheng CHANG, Holly Springs, NC (US)

(21) Appl. No.: 18/037,226

(22) PCT Filed: Dec. 2, 2021
(86) PCT No.: PCT/IB2021/061203

§ 371 Date: May 16, 2023

Related U.S. Application Data
(60) Provisional application No. 63/120,362, filed on Dec. 2, 2020.

(51) Int. Cl. A61K 39/145; C07K 14/11; A61K 39/215; C07K 14/165; A61P 31/16; A61K 39/00

(52) U.S. Cl. CPC A61K 39/145; C07K 14/11; A61K 39/215; C07K 14/165; A61P 31/16; A61K 2039/53; A61K 2039/572; A61K 2039/575

(57) ABSTRACT

The present disclosure relates to multicistronic RNA vaccines and uses thereof. The present disclosure also relates to multicistronic conventional mRNA vaccines and uses thereof. The present disclosure further relates to multicistronic self-replicating RNA vaccines and uses thereof

### RELATED APPLICATION DATA

The present application claims priority from U.S. Patent Application No. 63/120,362 filed 2 Dec. 2020 entitled “Multicistronic self-replicating RNA and uses thereof”, the entire contents of which is hereby incorporated by reference.

### SEQUENCE LISTING

The present application is filed together with a Sequence Listing in electronic form. The entire contents of the Sequence Listing are hereby incorporated by reference.

### FIELD

The present disclosure relates to multicistronic RNA vaccines and uses thereof. The present disclosure also relates to multicistronic conventional mRNA vaccines and uses thereof. The present disclosure further relates to multicistronic self-replicating RNA vaccines and uses thereof.

### BACKGROUND

Respiratory viral infections are a significant threat to human health and lives. Infections, such as those caused by the influenza virus and severe acute respiratory syndrome coronavirus (SARS-CoV) have been known to cause global pandemics, killing millions of people worldwide. More recently, SAR-CoV-2 has been responsible for causing the on-going worldwide pandemic of the severely infectious coronavirus disease 2019 (COVID-19).

Currently, infections such as influenza are treated with antivirals or other drugs. However, there are currently no specific and effective treatments for most respiratory viral infections. For those specific treatments available for some respiratory viral infection, for example, mRNA vaccines against COVID-19, further improvements can be made to increase their efficacy.

Viral vaccines, such as those for influenza, rely upon the induction of antibodies that protect against infection by neutralizing virions or blocking the virus's entry into cells. Humoral immune responses target viral surface proteins, however as these surface proteins are conserved within each strain, antibody-mediated protection is inadequate against strains with serologically distinct surface proteins. Furthermore, the surface proteins of many viruses are capable of rapid mutation. This means that most vaccines must be multivalent, i.e., include antigens from strains that are predicted to be most prevalent in a given time period.

To enhance the immune response (e.g., antibody response) mounted to the influenza virus surface proteins, various adjuvants and immuno-potentiating agents are included in the vaccine formulation. However, safety and efficacy issues remain.

Currently, egg-based manufacturing processes are the most common way that influenza vaccines are produced. This process requires a significant amount of time to optimize virus growth in the eggs, as well as resources (i.e., eggs) to produce sufficient amounts of vaccine, particularly during a pandemic. Furthermore, given the long development time required, vaccine strain selection is conducted before the vaccine is made available, making it difficult to respond to changes in the virus. Influenza vaccines have also been produced using cell-based manufacturing processes involving cultured mammalian cells (e.g. Madin-Darby Canine Kidney, or MDCK cells) in place of eggs, and viral-based platforms involving recombinant virus (e.g. baculovirus encoding an antigen of influenza).

There remains a need for the development of specific and efficient viral vaccines that can be produced more rapidly than current egg-based techniques, for the treatment or prevention of respiratory viral infections, such as influenza and COVID-19. Nucleic acid-based vaccines offer distinct advantages over the current egg-based manufacturing platform, although some challenges remain. For example, the inherently labile nature of mRNA results in most RNA-based vaccines having limited ability to provide antigen at a dose and duration required to produce a strong, durable immune response. Therefore, it will be apparent to the skilled person that there is a need in the art for an improved means for delivery of exogenous nucleic acids to a subject. There is also a need in the art for an mRNA vaccine with enhanced stability and improved expression of antigen(s) within the target cells of a subject.

### SUMMARY

The inventors of the present disclosure have identified a RNA that has improved activity and that permits efficient expression of more than one antigen (i.e., a multicistronic RNA). The present disclosure is based on the inventors' identification of a self-replicating RNA that has improved activity. In particular, the inventors have identified a self-replicating RNA that permits efficient expression of more than one antigen and does not result in the formation of unwanted fusion proteins.

The findings by the inventors provide the basis for a multicistronic RNA. The findings by the inventors also provide the basis for a multicistronic self-replicating RNA. Furthermore, the findings by the inventors provide the basis for a multicistronic conventional (i.e., non self-replicating) RNA. The findings by the inventors also provide the basis for methods of treating or preventing or delaying progression of a disease or disorder (e.g., a disease caused by a respiratory viral infection, such as influenza, a SARS-COV-2 infection, COVID-19 or ARDS) in a subject.

Accordingly, the present disclosure provides a polynucleotide comprising: a) a first nucleotide sequence encoding a first polypeptide of interest; and b) a second nucleotide sequence encoding a second polypeptide of interest operably linked to a regulatory element selected from the group consisting of a subgenomic (SG) promoter and an internal ribosome entry site (IRES).

In one example, the polynucleotide comprises, in order from 5′ to 3′: a) a first nucleotide sequence encoding a first polypeptide of interest; and b) a second nucleotide sequence encoding a second polypeptide of interest operably linked to a regulatory element selected from the group consisting of a SG promoter and an IRES.

The present disclosure also provides a polynucleotide comprising: a) a first nucleotide sequence encoding a first antigen of interest; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest; and b) a second nucleotide sequence encoding a second antigen of interest operably linked to a regulatory element selected from the group consisting of a SG promoter and an IRES.

In one example, the polynucleotide is RNA or DNA. For example, the polynucleotide is RNA. In one example, the polynucleotide is DNA.

In one example, the RNA is messenger RNA (mRNA). In one example, the mRNA is conventional mRNA (cRNA) or self-replicating mRNA.

Accordingly, the present disclosure provides a RNA comprising: a) a first nucleotide sequence encoding a first antigen of interest; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest; and b) a second nucleotide sequence encoding a second antigen of interest operably linked to a regulatory element selected from the group consisting of a SG promoter and an IRES.

The present disclosure also provides a cRNA comprising: a) a first nucleotide sequence encoding a first antigen of interest; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest; and b) a second nucleotide sequence encoding a second antigen of interest operably linked to a regulatory element selected from the group consisting of a SG promoter and an IRES.

The present disclosure further provides a self-replicating mRNA comprising: a) a first nucleotide sequence encoding a first antigen of interest; and b) a second nucleotide sequence encoding a second antigen of interest operably linked to a regulatory element selected from the group consisting of a SG promoter and an IRES.

In one example, the self-replicating mRNA comprises, in order from 5′ to 3′: a) a first nucleotide sequence encoding a first antigen of interest; and b) a second nucleotide sequence encoding a second antigen of interest operably linked to a regulatory element selected from the group consisting of a SG promoter and an IRES.

In one example, the first nucleotide sequence encoding the first antigen of interest is operably linked to a regulatory element. For example, the regulatory element is operably linked to the 5′ end of the first nucleotide sequence. In one example, the regulatory element is selected from the group consisting of a Kozak consensus sequence, an IRES, a SG promoter and combinations thereof. For example, the regulatory element is a Kozak consensus sequence. For example, the regulatory element is an IRES. For example, the regulatory element is a SG promoter.

In one example, the Kozak consensus sequence comprises or consists of a sequence set forth in SEQ ID NO: 38. In one example, the Kozak consensus sequence consists of a sequence set forth in SEQ ID NO: 38. In one example, the Kozak consensus sequence comprises a sequence set forth in SEQ ID NO: 38. For example, the Kozak consensus sequence is ACCATGG.

In one example, the Kozak consensus sequence comprises or consists of a sequence set forth in SEQ ID NO: 39. In one example, the Kozak consensus sequence consists of a sequence set forth in SEQ ID NO: 39. In one example, the Kozak consensus sequence comprises a sequence set forth in SEQ ID NO: 39. For example, the Kozak consensus sequence is ACCATG.

The present disclosure provides a polynucleotide comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

The present disclosure provides a RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

The present disclosure provides a cRNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

The present disclosure provides a self-replicating mRNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

In one example, the self-replicating mRNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a second antigen of interest operably linked to a regulatory element selected from the group consisting of a SG promoter and an IRES.

In one example, the first nucleotide sequence encoding the first antigen of interest is operably linked to a Kozak consensus sequence.

In one example, the first nucleotide sequence encoding the first antigen of interest is operably linked to a Kozak consensus sequence and a SG promoter. For example, the Kozak consensus sequence is operably linked to the 5′ end of the SG promoter which is operably linked to the 5′ end of the first nucleotide sequence encoding the first antigen of interest.

In one example, the first nucleotide sequence encoding the first antigen of interest is operably linked to a Kozak consensus sequence and an IRES. For example, the Kozak consensus sequence is operably linked to the 5′ end of the IRES which is operably linked to the 5′ end of the first nucleotide sequence encoding the first antigen of interest.

In one example, the first nucleotide sequence encoding the first antigen of interest is operably linked to a SG promoter.

In one example, the first nucleotide sequence encoding the first antigen of interest is operably linked to an IRES.

The present disclosure provides a polynucleotide comprising: a) a first nucleotide sequence encoding a first antigen of interest operably linked to a Kozak consensus sequence; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence; and b) a second nucleotide sequence encoding a second antigen of interest operably linked to a regulatory element selected from the group consisting of a SG promoter and an IRES.

The present disclosure provides a polynucleotide comprising: a) a first nucleotide sequence encoding a first antigen of interest operably linked to a Kozak consensus sequence and a SG promoter; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence and a SG promoter; and b) a second nucleotide sequence encoding a second antigen of interest operably linked to a regulatory element selected from the group consisting of a SG promoter and an IRES.

The present disclosure provides a polynucleotide comprising: a) a first nucleotide sequence encoding a first antigen of interest operably linked to a Kozak consensus sequence and an IRES; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence and an IRES; and b) a second nucleotide sequence encoding a second antigen of interest operably linked to a regulatory element selected from the group consisting of a SG promoter and an IRES.

The present disclosure provides a polynucleotide comprising: a) a first nucleotide sequence encoding a first antigen of interest operably linked to a SG promoter; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a SG promoter; and b) a second nucleotide sequence encoding a second antigen of interest operably linked to a regulatory element selected from the group consisting of a SG promoter and an IRES.

The present disclosure provides a polynucleotide comprising: a) a first nucleotide sequence encoding a first antigen of interest operably linked to an IRES; and b) a second nucleotide sequence encoding a second antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to an IRES; and b) a second nucleotide sequence encoding a second antigen of interest operably linked to a regulatory element selected from the group consisting of a SG promoter and an IRES.

The present disclosure provides a RNA comprising: a) a first nucleotide sequence encoding a first antigen of interest operably linked to a Kozak consensus sequence; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence; and b) a second nucleotide sequence encoding a second antigen of interest operably linked to a regulatory element selected from the group consisting of a SG promoter and an IRES.

The present disclosure provides a RNA comprising: a) a first nucleotide sequence encoding a first antigen of interest operably linked to a Kozak consensus sequence and a SG promoter; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence and a SG promoter; and b) a second nucleotide sequence encoding a second antigen of interest operably linked to a regulatory element selected from the group consisting of a SG promoter and an IRES.

The present disclosure provides a RNA comprising: a) a first nucleotide sequence encoding a first antigen of interest operably linked to a Kozak consensus sequence and an IRES; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence and an IRES; and b) a second nucleotide sequence encoding a second antigen of interest operably linked to a regulatory element selected from the group consisting of a SG promoter and an IRES.

The present disclosure provides a RNA comprising: a) a first nucleotide sequence encoding a first antigen of interest operably linked to a SG promoter; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a SG promoter; and b) a second nucleotide sequence encoding a second antigen of interest operably linked to a regulatory element selected from the group consisting of a SG promoter and an IRES.

The present disclosure provides a RNA comprising: a) a first nucleotide sequence encoding a first antigen of interest operably linked to an IRES; and b) a second nucleotide sequence encoding a second antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to an IRES; and b) a second nucleotide sequence encoding a second antigen of interest operably linked to a regulatory element selected from the group consisting of a SG promoter and an IRES.

The present disclosure provides a cRNA comprising: a) a first nucleotide sequence encoding a first antigen of interest operably linked to a Kozak consensus sequence; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence; and b) a second nucleotide sequence encoding a second antigen of interest operably linked to a regulatory element selected from the group consisting of a SG promoter and an IRES.

The present disclosure provides a cRNA comprising: a) a first nucleotide sequence encoding a first antigen of interest operably linked to a Kozak consensus sequence and a SG promoter; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence and a SG promoter; and b) a second nucleotide sequence encoding a second antigen of interest operably linked to a regulatory element selected from the group consisting of a SG promoter and an IRES.

The present disclosure provides a cRNA comprising: a) a first nucleotide sequence encoding a first antigen of interest operably linked to a Kozak consensus sequence and an IRES; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence and an IRES; and b) a second nucleotide sequence encoding a second antigen of interest operably linked to a regulatory element selected from the group consisting of a SG promoter and an IRES.

The present disclosure provides a cRNA comprising: a) a first nucleotide sequence encoding a first antigen of interest operably linked to a SG promoter; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a SG promoter; and b) a second nucleotide sequence encoding a second antigen of interest operably linked to a regulatory element selected from the group consisting of a SG promoter and an IRES.

The present disclosure provides a cRNA comprising: a) a first nucleotide sequence encoding a first antigen of interest operably linked to an IRES; and b) a second nucleotide sequence encoding a second antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to an IRES; and b) a second nucleotide sequence encoding a second antigen of interest operably linked to a regulatory element selected from the group consisting of a SG promoter and an IRES.

The present disclosure provides a multicistronic self-replicating RNA comprising: a) a first nucleotide sequence encoding a first antigen operably linked to a subgenomic (SG) promoter; and b) a second nucleotide sequences encoding a second antigen operably linked to a regulatory element selected from the group consisting of a SG promoter and an internal ribosome entry site (IRES).

In one example, the multicistronic self-replicating RNA of the present disclosure comprises, in order from 5′ to 3′, a) a first nucleotide sequence encoding a first antigen operably linked to a SG promoter; and b) a second nucleotide sequence encoding a second antigen operably linked to an IRES or a SG promoter.

In one example, the polynucleotide is a bicistronic RNA. For example, the polynucleotide is a bicistronic cRNA. In one example, the cRNA is a bicistronic cRNA. In another example, the polynucleotide is a bicistronic self-replicating mRNA. For example, the self-replicating RNA is a bicistronic self-replicating RNA.

In one example, the second nucleotide sequence encoding a second antigen is operably linked to an IRES.

In one example, the second nucleotide sequence encoding a second antigen is operably linked to a SG promoter.

In one example, the polynucleotide is a multicistronic RNA. For example, the polynucleotide is a multicistronic cRNA. For example, the cRNA is a multicistronic cRNA. In another example, the polynucleotide is a multicistronic self-replicating mRNA. For example, the self-replicating RNA is a multicistronic self-replicating mRNA.

In one example, the polynucleotide comprises one or more additional nucleotide sequences, wherein each sequence encodes an additional antigen of interest operably linked to a regulatory element selected from the group consisting of a SG promoter and an IRES, wherein the one or more nucleotide sequences are located 3′ of the second nucleotide sequence. For example, the polynucleotide comprises at least three nucleotide sequences, or at least four nucleotide sequences, or at least five nucleotide sequences, wherein each nucleotide sequence encodes an antigen.

In one example, the multicistronic RNA comprises one or more additional nucleotide sequences, wherein each sequence encodes an additional antigen of interest operably linked to a regulatory element selected from the group consisting of a SG promoter and an IRES, wherein the one or more nucleotide sequences are located 3′ of the second nucleotide sequence. For example, the multicistronic RNA comprises at least three nucleotide sequences, or at least four nucleotide sequences, or at least five nucleotide sequences, wherein each nucleotide sequence encodes an antigen.

In one example, the multicistronic cRNA comprises one or more additional nucleotide sequences, wherein each sequence encodes an additional antigen of interest operably linked to a regulatory element selected from the group consisting of a SG promoter and an IRES, wherein the one or more nucleotide sequences are located 3′ of the second nucleotide sequence. For example, the multicistronic cRNA comprises at least three nucleotide sequences, or at least four nucleotide sequences, or at least five nucleotide sequences, wherein each nucleotide sequence encodes an antigen.

In one example, the multicistronic self-replicating RNA comprises one or more additional nucleotide sequences, wherein each nucleotide sequence encodes an additional antigen operably linked to a regulatory element selected from the group consisting of a SG promoter and an IRES, and wherein the one or more nucleotide sequences encoding additional antigens are located 3′ of the second nucleotide sequence encoding the second antigen. For example, the multicistronic self-replicating RNA comprises at least three nucleotide sequences, or at least four nucleotide sequences, or at least five nucleotide sequences, wherein each nucleotide sequence encodes an antigen.

In one example, the polynucleotide comprises at least three nucleotide sequences, wherein each nucleotide sequence encodes an antigen.

In one example, the multicistronic RNA comprises at least three nucleotide sequences, wherein each nucleotide sequence encodes an antigen. For example, the RNA is a tricistronic RNA.

In one example, the multicistronic cRNA comprises at least three nucleotide sequences, wherein each nucleotide sequence encodes an antigen. For example, the cRNA is a tricistronic RNA.

In one example, the multicistronic self-replicating RNA comprises at least three nucleotide sequences, wherein each nucleotide sequence encodes an antigen. For example, the self-replicating RNA is a tricistronic self-replicating RNA.

In one example, the polynucleotide comprises, in order from 5′ to 3′, a) a first nucleotide sequence encoding a first antigen; b) a second nucleotide sequence encoding a second antigen operably linked to an IRES or a SG promoter; and c) a third nucleotide sequence encoding a third antigen operably linked to an IRES or a SG promoter.

In one example, the multicistronic RNA comprises, in order from 5′ to 3′, a) a first nucleotide sequence encoding a first antigen; b) a second nucleotide sequence encoding a second antigen operably linked to an IRES or a SG promoter; and c) a third nucleotide sequence encoding a third antigen operably linked to an IRES or a SG promoter.

In one example, the multicistronic cRNA comprises, in order from 5′ to 3′, a) a first nucleotide sequence encoding a first antigen; b) a second nucleotide sequence encoding a second antigen operably linked to an IRES or a SG promoter; and c) a third nucleotide sequence encoding a third antigen operably linked to an IRES or a SG promoter.

In one example, the multicistronic self-replicating mRNA comprises, in order from 5′ to 3′, a) a first nucleotide sequence encoding a first antigen; b) a second nucleotide sequence encoding a second antigen operably linked to an IRES or a SG promoter; and c) a third nucleotide sequence encoding a third antigen operably linked to an IRES or a SG promoter.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES or a SG promoter; and c) a third nucleotide sequence encoding a third antigen operably linked to an IRES or a SG promoter.

In one example, the polynucleotide comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to a SG promoter. In another example, the polynucleotide comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to an IRES.

In one example, the multicistronic RNA comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to an IRES; and c) a third nucleotide sequence encoding a third antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; and c) a third nucleotide sequence encoding a third antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; and c) a third nucleotide sequence encoding a third antigen operably linked to an IRES.

In another example, the polynucleotide comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to an IRES. In another example, the polynucleotide comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to a SG promoter.

In another example, the multicistronic RNA comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to an IRES. In another example, the multicistronic RNA comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to a SG promoter.

In another example, the multicistronic cRNA comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to an IRES. In another example, the multicistronic cRNA comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to a SG promoter.

In another example, the multicistronic self-replicating mRNA comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to an IRES. In another example, the multicistronic self-replicating mRNA comprises, in order from 5′ to 3′, a) a first nucleotide sequence encoding a first antigen; b) a second nucleotide sequence encoding a second antigen operably linked to a SG promoter; and c) a third nucleotide sequence encoding a third antigen operably linked to a SG promoter.

In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; and c) a third nucleotide sequence encoding a third antigen operably linked to an IRES. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; and c) a third nucleotide sequence encoding a third antigen operably linked to a SG promoter.

In one example, the polynucleotide comprises at least four nucleotide sequences, wherein each nucleotide sequence encodes an antigen.

In one example, the multicistronic RNA comprises at least four nucleotide sequences, wherein each nucleotide sequence encodes an antigen.

In one example, the multicistronic cRNA comprises at least four nucleotide sequences, wherein each nucleotide sequence encodes an antigen.

In one example, the multicistronic self-replicating RNA comprises at least four nucleotide sequences, wherein each nucleotide sequence encodes an antigen.

In one example, the polynucleotide comprises, in order from 5′ to 3′, a) a first nucleotide sequence encoding a first antigen; b) a second nucleotide sequence encoding a second antigen operably linked to an IRES or a SG promoter; c) a third nucleotide sequence encoding a third antigen operably linked to an IRES or a SG promoter; and d) a fourth nucleotide sequence encoding a fourth antigen operably linked to an IRES or a SG promoter.

In one example, the multicistronic RNA comprises, in order from 5′ to 3′, a) a first nucleotide sequence encoding a first antigen; b) a second nucleotide sequence encoding a second antigen operably linked to an IRES or a SG promoter; c) a third nucleotide sequence encoding a third antigen operably linked to an IRES or a SG promoter; and d) a fourth nucleotide sequence encoding a fourth antigen operably linked to an IRES or a SG promoter.

In one example, the multicistronic cRNA comprises, in order from 5′ to 3′, a) a first nucleotide sequence encoding a first antigen; b) a second nucleotide sequence encoding a second antigen operably linked to an IRES or a SG promoter; c) a third nucleotide sequence encoding a third antigen operably linked to an IRES or a SG promoter; and d) a fourth nucleotide sequence encoding a fourth antigen operably linked to an IRES or a SG promoter.

In one example, the multicistronic self-replicating mRNA comprises, in order from 5′ to 3′, a) a first nucleotide sequence encoding a first antigen; b) a second nucleotide sequence encoding a second antigen operably linked to an IRES or a SG promoter; c) a third nucleotide sequence encoding a third antigen operably linked to an IRES or a SG promoter; and d) a fourth nucleotide sequence encoding a fourth antigen operably linked to an IRES or a SG promoter.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES or a SG promoter; c) a third nucleotide sequence encoding a third antigen operably linked to an IRES or a SG promoter; and d) a fourth nucleotide sequence encoding a fourth antigen operably linked to an IRES or a SG promoter.

In one example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to a SG promoter. In another example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to a SG promoter. In a further example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to a SG promoter. In another example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to a SG promoter. In a further example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to a SG promoter. In a further example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to a SG promoter. In a further example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to a SG promoter. In a further example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to a SG promoter; and d) a fourth nucleotide sequence encoding a fourth antigen operably linked to an IRES.

In one example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to a SG promoter. In another example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to a SG promoter; and d) a fourth nucleotide sequence encoding a fourth antigen operably linked to an IRES.

In one example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to an IRES. In a further example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to an IRES. In a further example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to an IRES. In a further example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to an IRES. In a further example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to an IRES; and d) a fourth nucleotide sequence encoding a fourth antigen operably linked to an IRES. In a further example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to an IRES; and d) a fourth nucleotide sequence encoding a fourth antigen operably linked to an IRES.

In one example, the polynucleotide comprises at least five nucleotide sequences, wherein each nucleotide sequence encodes an antigen.

In one example, the multicistronic RNA comprises at least five nucleotide sequences, wherein each nucleotide sequence encodes an antigen.

In one example, the multicistronic cRNA comprises at least five nucleotide sequences, wherein each nucleotide sequence encodes an antigen.

In one example, the multicistronic self-replicating RNA comprises at least five nucleotide sequences, wherein each nucleotide sequence encodes an antigen.

In one example, the polynucleotide comprises, in order from 5′ to 3′, a) a first nucleotide sequence encoding a first antigen; b) a second nucleotide sequence encoding a second antigen operably linked to an IRES or SG promoter; c) a third nucleotide sequence encoding a third antigen operably linked to an IRES or a SG promoter; d) a fourth nucleotide sequence encoding a fourth antigen operably linked to an IRES or a SG promoter; and e) a fifth nucleotide sequence encoding a fifth antigen operably linked to an IRES or a SG promoter.

In one example, the multicistronic RNA comprises, in order from 5′ to 3′, a) a first nucleotide sequence encoding a first antigen; b) a second nucleotide sequence encoding a second antigen operably linked to an IRES or SG promoter; c) a third nucleotide sequence encoding a third antigen operably linked to an IRES or a SG promoter; d) a fourth nucleotide sequence encoding a fourth antigen operably linked to an IRES or a SG promoter; and e) a fifth nucleotide sequence encoding a fifth antigen operably linked to an IRES or a SG promoter.

In one example, the multicistronic cRNA comprises, in order from 5′ to 3′, a) a first nucleotide sequence encoding a first antigen; b) a second nucleotide sequence encoding a second antigen operably linked to an IRES or SG promoter; c) a third nucleotide sequence encoding a third antigen operably linked to an IRES or a SG promoter; d) a fourth nucleotide sequence encoding a fourth antigen operably linked to an IRES or a SG promoter; and e) a fifth nucleotide sequence encoding a fifth antigen operably linked to an IRES or a SG promoter.

In one example, the multicistronic self-replicating mRNA comprises, in order from 5′ to 3′, a) a first nucleotide sequence encoding a first antigen; b) a second nucleotide sequence encoding a second antigen operably linked to an IRES or SG promoter; c) a third nucleotide sequence encoding a third antigen operably linked to an IRES or a SG promoter; d) a fourth nucleotide sequence encoding a fourth antigen operably linked to an IRES or a SG promoter; and e) a fifth nucleotide sequence encoding a fifth antigen operably linked to an IRES or a SG promoter.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES or SG promoter; c) a third nucleotide sequence encoding a third antigen operably linked to an IRES or a SG promoter; d) a fourth nucleotide sequence encoding a fourth antigen operably linked to an IRES or a SG promoter; and e) a fifth nucleotide sequence encoding a fifth antigen operably linked to an IRES or a SG promoter.

In one example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In another example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In a further example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In a further example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In a further example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In a further example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to a SG promoter. In a further example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to a SG promoter. In a further example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to a SG promoter; d) a fourth nucleotide sequence encoding a fourth antigen operably linked to a SG promoter; and e) a fifth nucleotide sequence encoding a fifth antigen operably linked to an IRES.

In one example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to an IRES. In a further example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to an IRES. In a further example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to an IRES. In a further example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to an IRES. In a further example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to a SG promoter; and e) a fifth nucleotide sequence encoding a fifth antigen operably linked to an IRES. In a further example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to a SG promoter; d) a fourth nucleotide sequence encoding a fourth antigen operably linked to an IRES; and e) a fifth nucleotide sequence encoding a fifth antigen operably linked to an IRES.

In one example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the polynucleotide comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic RNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic cRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic self-replicating mRNA comprises, in order from 5′ to 3′, a) a first nucleotide sequence encoding a first antigen; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to a SG promoter. In another example, the multicistronic self-replicating RNA comprises, in order from 5′ to 3′, a) a first nucleotide sequence encoding a first antigen operably linked to a SG promoter; b) a second nucleotide sequence encoding a second antigen operably linked to a SG promoter; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the polynucleotide comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic RNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic cRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic self-replicating mRNA comprises, in order from 5′ to 3′, a) a first nucleotide sequence encoding a first antigen; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the multicistronic self-replicating RNA comprises, in order from 5′ to 3′, a) a first nucleotide sequence encoding a first antigen operably linked to a SG promoter; b) a second nucleotide sequence encoding a second antigen operably linked to an IRES; c) a third nucleotide sequence encoding a third antigen operably linked to an IRES; d) a fourth nucleotide sequence encoding a fourth antigen operably linked to an IRES; and e) a fifth nucleotide sequence encoding a fifth antigen operably linked to an IRES.

In one example, the SG promoter is a native SG promoter. For example, a native SG promoter is a promoter that is native to the RNA virus from which it is derived and/or based on (e.g., an alphavirus). In one example, the native SG promoter is a native alphavirus SG promoter.

In one example, the SG promoter is a minimal SG promoter or an extended SG promoter.

In one example, the SG promoter is a minimal SG promoter. In one example, the native SG promoter is a minimal SG promoter. For example, the minimal SG promoter is the minimal sequence required for initiation of transcription. In one example, the minimal native SG promoter is 49 nucleotides in length. In one example, the minimal SG promoter is 49 nucleotides in length. In one example, the minimal native SG promoter is encoded by a sequence comprising or consisting of a sequence set forth in SEQ ID NO: 1. In one example, the minimal SG promoter is encoded by a sequence comprising or consisting of a sequence set forth in SEQ ID NO: 1.

In one example, the SG promoter is an extended SG promoter. In one example, the native SG promoter is an extended SG promoter. For example, the extended SG promoter is extended at the 5′ end with nucleotides occurring in a sequence encoding a non-structural protein (e.g., NSP4) of the RNA virus (e.g., an alphavirus). In one example, the extended SG promoter is extended at the 5′ end with nucleotides occurring in a sequence encoding an alphavirus NSP4. The addition of nucleotides to the 5′ end of the SG promoter sequence did not interfere with expression of the non-structural protein and viral replicase, e.g., alphavirus NSP4.

Surprisingly, the inventors found that the number of nucleotides added to the 5′ end of the SG promoter to enhance expression is limited. For example, the inventors found that an SG promoter extended at the 5′ end by 52 nucleotides occurring in a sequence encoding a non-structural protein (e.g., an alphavirus NSP4) did not work. In particular, no antigen expression was detected when an SG promoter extended at the 5′ end by 52 nucleotides occurring in a sequence encoding a non-structural protein was used. Accordingly, in one example, the SG promoter is extended at the 5′ end by 51 or fewer nucleotides occurring in a sequence encoding a non-structural protein (e.g., an alphavirus NSP4). In one example, the extended SG promoter is a minimal SG promoter extended at the 5′ end by no more than 51 nucleotides occurring in a sequence encoding a non-structural protein (e.g., an alphavirus NSP4). In one example, the extended SG promoter is encoded by a sequence comprising or consisting of a sequence set forth in SEQ ID NO: 1 extended at the 5′ end by no more than 51 nucleotides occurring in a sequence encoding a non-structural protein (e.g., an alphavirus NSP4). For example, the extended SG promoter is no more than 100 nucleotides in length. In one example, the extended SG promoter is encoded by a sequence comprising or consisting of nucleotides 2 to 101 of SEQ ID NO: 15.

In one example, the SG promoter is extended at the 5′ end by about 5 nucleotides to about 20 nucleotides, for example by about 5 nucleotides, or about 10 nucleotides, or about 12, or about 15 nucleotides, or about 20 nucleotides, occurring in a sequence encoding a non-structural protein (e.g., an alphavirus NSP4). In another example, the SG promoter is extended at the 5′ end by about 20 to about 35 nucleotides, for example, by about 25 nucleotides or about 27 nucleotides, or about 30 nucleotides, or about 35 nucleotides, occurring in a sequence encoding a non-structural protein (e.g., an alphavirus NSP4).

In one example, the SG promoter is extended at the 5′ end by about 12 nucleotides occurring in a sequence encoding a non-structural protein (e.g., an alphavirus NSP4). In one example, the extended SG promoter is encoded by a sequence set forth in SEQ ID NO: 1 extended at the 5′ end by 12 nucleotides occurring in a sequence encoding a non-structural protein (e.g., an alphavirus NSP4). For example, the extended SG promoter is no more than 61 nucleotides in length. In one example, the extended SG promoter is encoded by a sequence comprising or consisting of nucleotides 41 to 101 of SEQ ID NO: 15. In another example, the extended SG promoter is encoded by a sequence comprising or consisting of a sequence set forth in SEQ ID NO: 2.

In one example, the SG promoter is extended at the 5′ end by about 31 nucleotides occurring in a sequence encoding a non-structural protein (e.g., an alphavirus NSP4). In one example, the extended SG promoter is encoded by a sequence set forth in SEQ ID NO: 1 extended at the 5′ end by 31 nucleotides occurring in a sequence encoding a non-structural protein (e.g., an alphavirus NSP4). For example, the extended SG promoter is no more than 80 nucleotides in length. In one example, the extended SG promoter is encoded by a sequence comprising or consisting of nucleotides 22 to 101 of SEQ ID NO: 15. In another example, the extended SG promoter is encoded by a sequence comprising or consisting of a sequence set forth in SEQ ID NO: 3.

In one example, the extended SG promoter comprises a repeat sequence corresponding to nucleotides 66 to 75 of SEQ ID NO: 15. For example, the extended SG promoter is encoded by a sequence comprising nucleotides 50 to 75 of SEQ ID NO: 15 and nucleotides 66 to 101 of SEQ ID NO: 15. For example, the extended SG promoter is encoded by a sequence set forth in SEQ ID NO: 47.

In one example, the IRES is a wild-type IRES derived from encephalomyocarditis virus (EMCV). For example, the wild-type EMCV IRES comprises a sequence set forth in SEQ ID NO: 4.

In one example, the first and/or second nucleotide sequence and/or the one or more additional nucleotide sequences are codon optimized.

In one example, the G/C content of the first and/or second nucleotide sequence and/or the one or more additional nucleotide sequences are modified.

In one example, the G/C content of the first and/or second nucleotide sequence and/or the one or more additional nucleotide sequences are increased by at least 5% compared to the G/C content of the unmodified sequence. For example, the G/C content of the first and/or second nucleotide sequence and/or the one or more additional nucleotide sequences are increased by at least 10%, or 15%, or 20%, or 25%, or 30%, or 35%, or 40% compared to the G/C content of the unmodified sequence.

In one example, the polynucleotide comprises at least one chemically modified nucleotide.

In one example, the chemically modified nucleotide is selected from the group consisting of N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (y), 1-methylpseudouridine (mly), and combinations thereof. For example, the chemically modified nucleotide is N6,2′-O-dimethyl-adenosine (m6Am). For example, the chemically modified nucleotide is 5-methyluridine (m5U). For example, the chemically modified nucleotide is N4-acetylcytidine (ac4C). For example, the chemically modified nucleotide is 2-thiocytidine (s2C). For example, the chemically modified nucleotide is 2-thiouridine (s2U). For example, the chemically modified nucleotide is 5-methylcytidine (m5C). For example, the chemically modified nucleotide is N6-methyladenosine (m6a). For example, the chemically modified nucleotide is pseudouridine (y). For example, the chemically modified nucleotide is 1-methylpseudouridine (mly).

In one example, the first nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 45.

In one example, the 5′-UTR, the fragment and/or the variant thereof is between and 2000 nucleotides in length. For example, the 5′-UTR, the fragment and/or the variant thereof is between 40 and 100 nucleotides in length. For example, the 5′-UTR, the fragment and/or the variant thereof is between 100 and 250 nucleotides in length. For example, the 5′-UTR, the fragment and/or the variant thereof is between 250 and 500 nucleotides in length. For example, the 5′-UTR, the fragment and/or the variant thereof is between 500 and 750 nucleotides in length. For example, the 5′-UTR, the fragment and/or the variant thereof is between 750 and 1000 nucleotides in length. For example, the 5′-UTR, the fragment and/or the variant thereof is between 1000 and 1250 nucleotides in length. For example, the 5′-UTR, the fragment and/or the variant thereof is between 1250 and 1500 nucleotides in length. For example, the 5′-UTR, the fragment and/or the variant thereof is between 1500 and 1750 nucleotides in length. For example, the 5′-UTR, the fragment and/or the variant thereof is between 1750 and 2000 nucleotides in length.

In one example, the 5′-UTR, the fragment and/or the variant thereof comprises a nucleotide sequence at least 90% identical to a nucleotide sequence set forth in any one of SEQ ID NO: 40 to 54. For example, the 5′-UTR, the fragment and/or the variant thereof comprises a nucleotide sequence 90%, or 91%, or 92%, or 93%, or 94%, or 95%, or 96%, or 97%, or 98%, or 99% identical to a nucleotide sequence set forth in any one of SEQ ID NO: 40 to 54.

In one example, the polynucleotide comprises a combination of two or more 5′-UTRs, fragments and/or variants thereof. In one example, the two or more 5′-UTRs are the same. In one example, the two or more 5′-UTRs are different.

In one example, the nucleotide sequence comprising the 5′UTR comprises at least one microRNA binding site, an AU rich element (ARE), a GC-rich element, a stem loop, and combinations thereof. In one example, the nucleotide sequence comprises a microRNA binding site. In one example, the nucleotide sequence comprises an AU rich element (ARE). In one example, the nucleotide comprises a GC-rich element. In one example, the nucleotide sequence comprises a stem loop. For example, the stem loop is a histone stem loop.

In one example, the polynucleotide further comprises a nucleotide sequence comprising a 3′UTR. In one example, the nucleotide sequence comprising the 3′UTR is located 3′ of the second or the one or more additional nucleotide sequences. For example, the nucleotide sequence comprising the 3′UTR is located 3′ of the second nucleotide sequence. In one example, the 3′UTR comprises a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 46.

In one example, the 3′UTR, the fragment and/or the variant thereof is between 40 and 400 nucleotides in length. For example, the 3′-UTR is between 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300, or 300 and 325, or 325 and 350, or 350 and 375, or 375 and 400 nucleotides in length. For example, the 3′-UTR, the fragment and/or the variant thereof is between and 50 nucleotides in length. For example, the 3′-UTR, the fragment and/or the variant thereof is between 50 and 60 nucleotides in length. For example, the 3′-UTR, the fragment and/or the variant thereof is between 60 and 70 nucleotides in length. For example, the 3′-UTR, the fragment and/or the variant thereof is between 70 and 80 nucleotides in length. For example, the 3′-UTR, the fragment and/or the variant thereof is between 80 and 90 nucleotides in length. For example, the 3′-UTR, the fragment and/or the variant thereof is between 90 and 100 nucleotides in length. For example, the 3′-UTR, the fragment and/or the variant thereof is between 100 and 125 nucleotides in length. For example, the 3′-UTR, the fragment and/or the variant thereof is between 125 and 150 nucleotides in length. For example, the 3′-UTR, the fragment and/or the variant thereof is between 150 and 175 nucleotides in length. For example, the 3′-UTR, the fragment and/or the variant thereof is between 175 and 200 nucleotides in length. For example, the 3′-UTR, the fragment and/or the variant thereof is between 200 and 225 nucleotides in length. For example, the 3′-UTR, the fragment and/or the variant thereof is between 225 and 250 nucleotides in length. For example, the 3′-UTR, the fragment and/or the variant thereof is between 250 and 275 nucleotides in length. For example, the 3′-UTR, the fragment and/or the variant thereof is between 275 and 300 nucleotides in length. For example, the 3′-UTR, the fragment and/or the variant thereof is between 300 and 325 nucleotides in length. For example, the 3′-UTR, the fragment and/or the variant thereof is between 325 and 350 nucleotides in length. For example, the 3′-UTR, the fragment and/or the variant thereof is between 350 and 375 nucleotides in length. For example, the 3′-UTR, the fragment and/or the variant thereof is between 375 and 400 nucleotides in length.

In one example, the polynucleotide comprises a combination of two or more 3′-UTRs, fragments and/or variants thereof. In one example, the two or more 3′-UTRs are the same. In one example, the two or more 3′-UTRs are different.

In one example, the nucleotide sequence comprising the 3′UTR, the fragment and/or variant thereof comprises at least one microRNA binding site, an AU rich element (ARE), a GC-rich element, a triple helix, a stem loop, one or more stop codons and combinations thereof. In one example, the nucleotide sequence comprises a microRNA binding site. In one example, the nucleotide sequence comprises an AU rich element (ARE). In one example, the nucleotide sequence comprises a GC-rich element. In one example, the nucleotide sequence comprises a triple helix. In one example, the nucleotide sequence comprises a stem loop. For example, the stem loop is a histone stem loop. In one example, the nucleotide sequence comprises one or more stop codons. For example, the one or more stop codons are located at the 5′end of the 3′-UTR.

In one example, the polynucleotide comprises a nucleotide sequence comprising one or more 3′ tailing sequences located at the 3′end of the nucleotide sequence comprising the 3′UTR. In one example, the one or more 3′ tailing sequences are selected from the group consisting of a poly-A sequence, polyadenylation signal, a G-quadruplex, a poly-C sequence, a stem loop and combinations thereof. For example, the 3′ tailing sequence comprises a poly-A sequence. In one example, the 3′ tailing sequence comprises a polyadenylation signal. In one example, the 3′ tailing sequence comprises a G-quadruplex. In one example, the 3′ tailing sequence comprises a poly-C sequence. In one example, the 3′ tailing sequence comprises a stem loop. For example, the stem loop is a histone stem loop. In one example, the 3′ tailing sequence comprises a poly-A sequence and a G-quadruplex. In one example, the 3′ tailing sequence comprises a stem loop (e.g., a histone stem loop) and a poly-A sequence.

In one example, the one or more 3′ tailing sequences comprises one or more poly-A sequences each comprising between 10 and 300 consecutive adenosine nucleotides. For example, the poly-A sequences each comprises between 10 and 20, or 20 and 30, or and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive adenosine nucleotides. For example, the one or more poly-A sequences each comprises between 10 and 20 consecutive adenosine nucleotides. For example, the one or more poly-A sequences each comprises between 20 and 30 consecutive adenosine nucleotides. For example, the one or more poly-A sequences each comprises between 30 and 40 consecutive adenosine nucleotides. For example, the one or more poly-A sequences each comprise 36 consecutive adenosine nucleotides. For example, the one or more poly-A sequences each comprises between 40 and 50 consecutive adenosine nucleotides. For example, the one or more poly-A sequences each comprises between 50 and 60 consecutive adenosine nucleotides. For example, the one or more poly-A sequences each comprises between 60 and 70 consecutive adenosine nucleotides. For example, the one or more poly-A sequences each comprises between 70 and 80 consecutive adenosine nucleotides. For example, the one or more poly-A sequences each comprises between 80 and 90 consecutive adenosine nucleotides. For example, the one or more poly-A sequences each comprises between 90 and 100 consecutive adenosine nucleotides. For example, the one or more poly-A sequences each comprises between 100 and 125 consecutive adenosine nucleotides. For example, the one or more poly-A sequences each comprises between 125 and 150 consecutive adenosine nucleotides. For example, the one or more poly-A sequences each comprises between 150 and 175 consecutive adenosine nucleotides. For example, the one or more poly-A sequences each comprises between 175 and 200 consecutive adenosine nucleotides. For example, the one or more poly-A sequences each comprises between 200 and 225 consecutive adenosine nucleotides. For example, the one or more poly-A sequences each comprises between 225 and 250 consecutive adenosine nucleotides. For example, the one or more poly-A sequences each comprises between 250 and 275 consecutive adenosine nucleotides. For example, the one or more poly-A sequences each comprises between 275 and 300 consecutive adenosine nucleotides.

In one example, the one or more poly-A sequence each comprises 10, or 20, or 30, or 40, or 50, or 60, or 70, or 80, or 90, or 100, or 125, or 150, or 175, or 200, or 225, or 250, or 275, or 300 consecutive adenosine nucleotides. For example, the one or more poly-A sequence each comprises 10 consecutive adenosine nucleotides. For example, the one or more poly-A sequence each comprises 20 consecutive adenosine nucleotides. For example, the one or more poly-A sequence each comprises 30 consecutive adenosine nucleotides. For example, the one or more poly-A sequence each comprises 40 consecutive adenosine nucleotides. For example, the one or more poly-A sequence each comprises 50 consecutive adenosine nucleotides. For example, the one or more poly-A sequence each comprises 60 consecutive adenosine nucleotides. For example, the one or more poly-A sequence each comprises 70 consecutive adenosine nucleotides. For example, the one or more poly-A sequence each comprises 80 consecutive adenosine nucleotides. For example, the one or more poly-A sequence each comprises 90 consecutive adenosine nucleotides. For example, the one or more poly-A sequence each comprises 100 consecutive adenosine nucleotides. For example, the one or more poly-A sequence each comprises 125 consecutive adenosine nucleotides. For example, the one or more poly-A sequence each comprises 150 consecutive adenosine nucleotides. For example, the one or more poly-A sequence each comprises 175 consecutive adenosine nucleotides. For example, the one or more poly-A sequence each comprises 200 consecutive adenosine nucleotides. For example, the one or more poly-A sequence each comprises 225 consecutive adenosine nucleotides. For example, the one or more poly-A sequence each comprises 250 consecutive adenosine nucleotides. For example, the one or more poly-A sequence each comprises 275 consecutive adenosine nucleotides. For example, the one or more poly-A sequence each comprises 300 consecutive adenosine nucleotides.

In one example, the poly-A sequence comprises 36 consecutive adenosine nucleotides. For example, the poly-A sequence comprises a sequence set forth in SEQ ID NO: 48.

In one example, the one or more poly-A sequences is separated by an interrupting linker. For example, the 3′tailing sequence comprises, in order of 5′ to 3′: a poly-A sequence comprising consecutive adenosine nucleotides, an interrupting linker, and a further poly-A sequence comprising consecutive adenosine nucleotides.

In one example, the interrupting linker is from 10 to 50, or 50 to 100, or 100 to 150 nucleotides in length. For example, the interrupting linker is from 10 to 50 nucleotides in length. For example, the interrupting linker is from 50 to 100 nucleotides in length. For example, the interrupting linker is from 100 to 150 nucleotides in length.

In one example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length. For example, the interrupting linker is 1 nucleotide in length. For example, the interrupting linker is 2 nucleotides in length. For example, the interrupting linker is 3 nucleotides in length. For example, the interrupting linker is 4 nucleotides in length. For example, the interrupting linker is 5 nucleotides in length. For example, the interrupting linker is 6 nucleotides in length. For example, the interrupting linker is 7 nucleotides in length. For example, the interrupting linker is 8 nucleotides in length. For example, the interrupting linker is 9 nucleotides in length. For example, the interrupting linker is 10 nucleotides in length. For example, the interrupting linker is 11 nucleotides in length. For example, the interrupting linker is 12 nucleotides in length. For example, the interrupting linker is 13 nucleotides in length. For example, the interrupting linker is 14 nucleotides in length. For example, the interrupting linker is 15 nucleotides in length. For example, the interrupting linker is 16 nucleotides in length. For example, the interrupting linker is 17 nucleotides in length. For example, the interrupting linker is 18 nucleotides in length. For example, the interrupting linker is 19 nucleotides in length. For example, the interrupting linker is 20 nucleotides in length. For example, the interrupting linker is 25 nucleotides in length. For example, the interrupting linker is 30 nucleotides in length. For example, the interrupting linker is 35 nucleotides in length. For example, the interrupting linker is 40 nucleotides in length. For example, the interrupting linker is 45 nucleotides in length. For example, the interrupting linker is 50 nucleotides in length. For example, the interrupting linker is 55 nucleotides in length. For example, the interrupting linker is 60 nucleotides in length. For example, the interrupting linker is 65 nucleotides in length. For example, the interrupting linker is 70 nucleotides in length. For example, the interrupting linker is 75 nucleotides in length. For example, the interrupting linker is 80 nucleotides in length. For example, the interrupting linker is 85 nucleotides in length. For example, the interrupting linker is 90 nucleotides in length. For example, the interrupting linker is 95 nucleotides in length. For example, the interrupting linker is 100 nucleotides in length. For example, the interrupting linker is 110 nucleotides in length. For example, the interrupting linker is 120 nucleotides in length. For example, the interrupting linker is 130 nucleotides in length. For example, the interrupting linker is 140 nucleotides in length. For example, the interrupting linker is 150 nucleotides in length.

In one example, the interrupting linker is 10 nucleotides in length. In one example, the interrupting linker comprises or consists of the nucleotide sequence set forth in SEQ ID NO: 40. For example, the interrupting linker comprises or consists of a nucleotide sequence GCAUAUGACU.

In one example, the 3′ tailing sequence comprises, in order of 5′ to 3′: a poly-A sequence comprising 30 consecutive adenosine nucleotides, an interrupting linker of 10 nucleotides, and a further poly-A sequence comprising 70 consecutive adenosine nucleotides.

In one example, the 3′ tailing sequence comprises, in order of 5′ to 3′: a poly-A sequence comprising 30 consecutive adenosine nucleotides, an interrupting linker comprising or consisting of the nucleotide sequence set forth in SEQ ID NO: 40, and a further poly-A sequence comprising 70 consecutive adenosine nucleotides.

In one example, the polynucleotide comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) the first nucleotide sequence encoding the first polypeptide of interest;
d) the second nucleotide sequence encoding the second polypeptide of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) the first nucleotide sequence encoding the first polypeptide of interest;
d) the second nucleotide sequence encoding the second polypeptide of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the cRNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) the first nucleotide sequence encoding the first polypeptide of interest;
d) the second nucleotide sequence encoding the second polypeptide of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the sa-mRNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) the first nucleotide sequence encoding the first polypeptide of interest;
d) the second nucleotide sequence encoding the second polypeptide of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the multicistronic self-replicating RNA of the present disclosure comprises, in order from 5′ to 3′:
a) a first nucleotide sequence encoding a first antigen operably linked to a minimal SG promoter; and a second nucleotide sequence encoding a second antigen operably linked to a minimal SG promoter; or
b) a first nucleotide sequence encoding a first antigen operably linked to a minimal SG promoter; and a second nucleotide sequence encoding a second antigen operably linked to an extended SG promoter; or
c) a first nucleotide sequence encoding a first antigen operably linked to a minimal SG promoter; and a second nucleotide sequence encoding a second antigen operably linked to an a wild-type EMCV IRES.

In one example, the multicistronic self-replicating RNA of the present disclosure comprises, in order from 5′ to 3′: a first nucleotide sequence encoding a first antigen operably linked to a minimal SG promoter; and a second nucleotide sequence encoding a second antigen operably linked to a minimal SG promoter.

For example, the multicistronic self-replicating RNA of the present disclosure comprises, in order from 5′ to 3′: a first nucleotide sequence encoding a first antigen operably linked to a minimal SG promoter comprising a sequence set forth in SEQ ID NO: 1; and a second nucleotide sequence encoding a second antigen operably linked to a minimal SG promoter comprising a sequence set forth in SEQ ID NO: 1.

In one example, the multicistronic self-replicating RNA of the present disclosure comprises, in order from 5′ to 3′: a first nucleotide sequence encoding a first antigen operably linked to a minimal SG promoter; and a second nucleotide sequence encoding a second antigen operably linked to an extended SG promoter.

In one example, the multicistronic self-replicating RNA of the present disclosure comprises, in order from 5′ to 3′: a first nucleotide sequence encoding a first antigen operably linked to a minimal SG promoter encoded by a sequence set forth in SEQ ID NO: 1; and a second nucleotide sequence encoding a second antigen operably linked to an extended SG promoter encoded by a sequence set forth in SEQ ID NO: 2.

In one example, the multicistronic self-replicating RNA of the present disclosure comprises, in order from 5′ to 3′: a first nucleotide sequence encoding a first antigen operably linked to a minimal SG promoter encoded by a sequence set forth in SEQ ID NO: 1; and a second nucleotide sequence encoding a second antigen operably linked to an extended SG promoter encoded by a sequence set forth in SEQ ID NO: 3.

In one example, the multicistronic self-replicating RNA of the present disclosure comprises, in order from 5′ to 3′: a first nucleotide sequence encoding a first antigen operably linked to a minimal SG promoter; and a second nucleotide sequence encoding a second antigen operably linked to a wild-type EMCV IRES.

In one example, the multicistronic self-replicating RNA of the present disclosure comprises, in order from 5′ to 3′: a first nucleotide sequence encoding a first antigen operably linked to a minimal SG promoter encoded by a sequence set forth in SEQ ID NO: 1; and a second nucleotide sequence encoding a second antigen operably linked to a wild-type EMCV IRES encoded by a sequence set forth in SEQ ID NO: 4.

In one example, the RNA further comprises a 5′ terminal cap structure.

In one example, the 5′ terminal cap structure is an endogenous cap or analogue thereof. For example, the 5′terminal cap structure is an endogenous cap. For example, the 5′terminal cap structure is an analogue of an endogenous cap.

In one example, the 5′ terminal cap structure comprise a guanine or guanine analogue thereof. For example, the 5′ terminal cap structure comprise a guanine. For example, the 5′ terminal cap structure comprise a guanine analogue of a guanine.

In one example, the 5′ terminal cap structure is selected from a group consisting of anti-reverse cap analogue (ARCA), N7,2′-0-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2. For example, the 5′ terminal cap structure is anti-reverse cap analogue (ARCA). For example, the 5′ terminal cap structure is N7,2′-0-dimethyl-guanosine (mCAP). For example, the 5′ terminal cap structure is inosine. For example, the 5′ terminal cap structure is N1-methyl-guanosine. For example, the 5′ terminal cap structure is 2′fluoro-guanosine. For example, the 5′ terminal cap structure is 7-deaza-guanosine. For example, the 5′ terminal cap structure is 8-oxo-guanosine. For example, the 5′ terminal cap structure is 2-amino-guanosine. For example, the 5′ terminal cap structure is LNA-guanosine. For example, the 5′ terminal cap structure is 2-azido-guanosine. For example, the 5′ terminal cap structure is N6,2′-O-dimethyladenosine. For example, the 5′ terminal cap structure is 7-methylguanosine (m7G). For example, the 5′ terminal cap structure is Cap1. For example, the 5′ terminal cap structure is Cap2.

In one example, the 5′terminal cap structure is linked to the 5′ end of the RNA by a 5′-5′-triphosphate linkage or a 5′-5′ phosphorothioate linkage. For example, the 5′terminal cap structure is linked to the 5′ end of the RNA by a 5′-5′-triphosphate linkage. For example, the 5′terminal cap structure is linked to the 5′ end of the RNA by a 5′-5′ phosphorothioate linkage.

In one example, the antigens are expressed at substantially the same level. For example, the antigens have a level of expression within about 10%, or about 5% or about 1% of each other. In another example, the antigens are expressed at different levels. For example, the antigens have a level of expression greater than about 10%, or about 15% or about 20% of each other. Methods for determining the level of expression are known in the art and/or are described herein.

In one example, the self-replicating RNA is from an alphavirus. For example, the alphavirus is selected from the group consisting of Semliki Forest virus (SFV), Sindbis virus (SIN), and Venezuelan equine encephalitis virus (VEE) and combinations thereof.

In one example, the self-replicating RNA is from a Semliki Forest virus (SFV).

In one example, the self-replicating RNA is from a Sindbis virus (SIN).

In one example, the self-replicating RNA is from a Venezuelan equine encephalitis virus (VEE).

In one example, the antigens are viral antigens. For example, the viral antigens are from a respiratory virus. In one example, the respiratory virus is selected from the group consisting of influenza virus, respiratory syncytial virus, parainfluenza viruses, metapneumovirus, rhinovirus, coronaviruses, adenoviruses and bocaviruses.

In one example, the viral antigens are from an influenza virus.

In one example, the viral antigens are from a respiratory syncytial virus.

In one example, the viral antigens are from a parainfluenza virus.

In one example, the viral antigens are from a metapneumovirus.

In one example, the viral antigens are from a rhinovirus.

In one example, the viral antigens are from a coronavirus.

In one example, the viral antigens are from an adenovirus.

In one example, the viral antigens are from a bocavirus.

In one example, the antigens are viral antigens from an influenza virus or a coronavirus.

In one example, the antigens are from a single strain of an influenza virus (i.e., monovalent) or from multiple strains (i.e., multivalent). For example, the self-replicating RNA includes antigens from one or more (e.g., 1 or 2 or 3) influenza virus strains.

In one example, the first and second influenza viral antigens are from different strains of the influenza virus. For example, the first and second antigens are from an influenza A, B and/or C virus strain.

In one example, the antigens are from an influenza A virus strain. For example, the antigens are an influenza A virus hemagglutinin (HA) protein, a neuraminidase (NA) protein, a matrix (M) protein, a nucleoprotein (NP), a non-structural (NS) protein, or an immunogenic fragment or variant thereof. In one example, the antigens are an influenza A hemagglutinin (HA) subtype H1, H2, H3, H4, H5, H6, H7, H8, H9, H10, H11, H12, H13, H14, H15 or H16 and/or an influenza A neuraminidase (NA) subtype N1, N2, N3, N4, N5, N6, N7, N8 or N9 and/or an influenza A matrix (M) protein subtype M1 or M2 and/or an influenza A non-structural (NS) protein subtype NS1 or NS2.

In one example, the influenza viral antigens are from different subtypes of the influenza virus. For example, different hemagglutinin subtypes and/or different neuraminidase subtypes and/or matrix protein subtypes, and/or nucleoprotein subtypes and/or non-structural protein subtypes.

The skilled person will be aware that pandemic strains of the influenza virus are commonly H1, H2, H3, H5, H6, H7 or H9 subtype influenza A virus strains. For example, H1N1, H2N2, H3N2, H5N1, H5N3, H6N1, H7N2, H7N3, H7N7, H7N9 and H9N2, strains.

In one example, the antigens are from influenza A virus strains having the same hemagglutinin subtypes. In another example, the antigens are influenza A virus strains having different hemagglutinin subtypes. In one example, the antigens are H1, H2, H3, H5, H6, H7 or H9 subtype influenza A virus strains. For example, the antigens are a H1 hemagglutinin, or a H2 hemagglutinin, or a H3 hemagglutinin, or a H5 hemagglutinin, or a H6 hemagglutinin, or a H7 hemagglutinin or a H9 hemagglutinin. For example, the antigens are a H5 subtype influenza A virus strain (i.e., a H5 hemagglutinin). In one example, the H5 hemagglutinin is an A/turkey/Turkey/i/2005 virus strain. For example, the H5 hemagglutinin is encoded by a sequence set forth in SEQ ID NO: 5. In one example, the H3 hemagglutinin is an A/Delaware/39/2019 virus strain. For example, the H3 hemagglutinin is encoded by a sequence set forth in SEQ ID NO: 54.

In one example, the antigens are influenza A virus strains having the same neuraminidase subtypes. In another example, the antigens are influenza A virus strains having different neuraminidase subtypes. In one example, the antigens are N1, N2, N3, N7 or N9 subtype influenza A virus strains. For example, the antigens are a N1 neuraminidase, or a N2 neuraminidase, or a N3 neuraminidase, or a N7 neuraminidase, or a N9 neuraminidase. For example, the antigens are a N1 neuraminidase subtype influenza A virus strain. In one example, the N1 neuraminidase is an A/turkey/Turkey/i/2005 strain. For example, the N1 neuraminidase is encoded by a sequence set forth in SEQ ID NO: 6. In one example, the N2 neuraminidase is an A/Delaware/39/2019 virus strain. For example, the N2 neuraminidase is encoded by a sequence set forth in SEQ ID NO: 55

In one example, the antigens are a H5 hemagglutinin protein and/or a N1 neuraminidase protein. For example, the first antigen is a H5 hemagglutinin subtype influenza A virus strain and the second antigen is a N1 neuraminidase subtype influenza A virus strain. In one example, the first antigen is a H5 hemagglutinin subtype influenza A virus strain encoded by a sequence set forth in SEQ ID NO: 5 and the second antigen is a N1 neuraminidase subtype influenza A virus strain encoded by a sequence set forth in SEQ ID NO: 6.

In one example, the present disclosure provides a polynucleotide, wherein the polynucleotide comprises, in order from 5′ to 3′:
a) a first nucleotide sequence encoding a H5 hemagglutinin protein; and a second nucleotide sequence encoding a N1 neuraminidase protein; or
b) a first nucleotide sequence encoding a N1 neuraminidase protein; and a second nucleotide sequence encoding a H5 hemagglutinin protein.

In one example, the present disclosure provides a polynucleotide, wherein the polynucleotide comprises, in order from 5′ to 3′, a first nucleotide sequence encoding a H5 hemagglutinin protein; and a second nucleotide sequence encoding a N1 neuraminidase protein.

In one example, the present disclosure provides a polynucleotide, wherein the polynucleotide comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; and a second nucleotide sequence encoding a N1 neuraminidase protein operably linked to a regulatory element selected from the group consisting of a SG promoter and an IRES.

In one example, the present disclosure provides a polynucleotide, wherein the polynucleotide comprises, in order from 5′ to 3′, a first nucleotide sequence encoding a N1 neuraminidase protein; and a second nucleotide sequence encoding a H5 hemagglutinin protein.

In one example, the present disclosure provides a polynucleotide, wherein the polynucleotide comprises, in order from 5′ to 3′, a first nucleotide sequence encoding a N1 neuraminidase protein operably linked to a regulatory element selected from the group consisting of a Kozak consensus sequence, an IRES, a SG promoter and combinations thereof; and a second nucleotide sequence encoding a H5 hemagglutinin protein operably linked to a regulatory element selected from the group consisting of a SG promoter and an IRES.

In one example, the present disclosure provides a RNA, wherein the RNA comprises, in order from 5′ to 3′:
a) a first nucleotide sequence encoding a H5 hemagglutinin protein; and a second nucleotide sequence encoding a N1 neuraminidase protein; or
b) a first nucleotide sequence encoding a N1 neuraminidase protein; and a second nucleotide sequence encoding a H5 hemagglutinin protein.

In one example, the present disclosure provides a RNA, wherein the RNA comprises, in order from 5′ to 3′, a first nucleotide sequence encoding a H5 hemagglutinin protein; and a second nucleotide sequence encoding a N1 neuraminidase protein.

In one example, the present disclosure provides a RNA, wherein the RNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; and a second nucleotide sequence encoding a N1 neuraminidase protein operably linked to a regulatory element selected from the group consisting of a SG promoter and an IRES.

In one example, the present disclosure provides a RNA, wherein the RNA comprises, in order from 5′ to 3′, a first nucleotide sequence encoding a N1 neuraminidase protein; and a second nucleotide sequence encoding a H5 hemagglutinin protein.

In one example, the present disclosure provides a RNA, wherein the RNA comprises, in order from 5′ to 3′, a first nucleotide sequence encoding a N1 neuraminidase protein operably linked to a regulatory element selected from the group consisting of a Kozak consensus sequence, an IRES, a SG promoter and combinations thereof; and a second nucleotide sequence encoding a H5 hemagglutinin protein operably linked to a regulatory element selected from the group consisting of a SG promoter and an IRES.

In one example, the present disclosure provides a cRNA, wherein the cRNA comprises, in order from 5′ to 3′:
a) a first nucleotide sequence encoding a H5 hemagglutinin protein; and a second nucleotide sequence encoding a N1 neuraminidase protein; or
b) a first nucleotide sequence encoding a N1 neuraminidase protein; and a second nucleotide sequence encoding a H5 hemagglutinin protein.

In one example, the present disclosure provides a cRNA, wherein the cRNA comprises, in order from 5′ to 3′, a first nucleotide sequence encoding a H5 hemagglutinin protein; and a second nucleotide sequence encoding a N1 neuraminidase protein.

In one example, the present disclosure provides a cRNA, wherein the cRNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; and a second nucleotide sequence encoding a N1 neuraminidase protein operably linked to a regulatory element selected from the group consisting of a SG promoter and an IRES.

In one example, the present disclosure provides a cRNA, wherein the cRNA comprises, in order from 5′ to 3′, a first nucleotide sequence encoding a N1 neuraminidase protein; and a second nucleotide sequence encoding a H5 hemagglutinin protein.

In one example, the present disclosure provides a cRNA, wherein the cRNA comprises, in order from 5′ to 3′, a first nucleotide sequence encoding a N1 neuraminidase protein operably linked to a regulatory element selected from the group consisting of a Kozak consensus sequence, an IRES, a SG promoter and combinations thereof; and a second nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein; and a second nucleotide sequence encoding a N1 neuraminidase protein; or
b) a first nucleotide sequence encoding a N1 neuraminidase protein; and a second nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a H5 hemagglutinin protein; and a second nucleotide sequence encoding a N1 neuraminidase protein.

In one example, the present disclosure provides a multicistronic self-replicating RNA, wherein the RNA comprises, in order from 5′ to 3′, a first nucleotide sequence encoding a H5 hemagglutinin protein operably linked to a SG promoter; and a second nucleotide sequence encoding a N1 neuraminidase protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1; and
b) a second nucleotide sequence encoding a N1 neuraminidase protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1; and
b) a second nucleotide sequence encoding a N1 neuraminidase protein operably linked to an extended SG promoter encoded by a sequence set forth in SEQ ID NO: 2.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1; and
b) a second nucleotide sequence encoding a N1 neuraminidase protein operably linked to an extended SG promoter encoded by a sequence set forth in SEQ ID NO: 3.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1; and
b) a second nucleotide sequence encoding a N1 neuraminidase protein operably linked to an IRES encoded by a sequence set forth in SEQ ID NO: 4.

In one example, the present disclosure provides a multicistronic self-replicating RNA, wherein the RNA comprises, in order from 5′ to 3′, a first nucleotide sequence encoding a N1 neuraminidase protein; and a second nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a N1 neuraminidase protein operably linked to a SG promoter; and a second nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a N1 neuraminidase protein operably linked to a minimal SG promoter encoded by a sequence set forth in SEQ ID NO: 1; and
b) a second nucleotide sequence encoding a H5 hemagglutinin protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′:
a) a first nucleotide sequence encoding a N1 neuraminidase protein operably linked to a minimal SG promoter encoded by a sequence set forth in SEQ ID NO: 1; and
b) a second nucleotide sequence encoding a H5 hemagglutinin protein operably linked to an extended SG promoter encoded by a sequence set forth in SEQ ID NO: 2.

In one example, the present disclosure provides a multicistronic self-replicating RNA, wherein the RNA comprises, in order from 5′ to 3′:
a) a first nucleotide sequence encoding a N1 neuraminidase protein operably linked to a minimal SG promoter encoded by a sequence set forth in SEQ ID NO: 1; and
b) a second nucleotide sequence encoding a H5 hemagglutinin protein operably linked to an extended SG promoter encoded by a sequence set forth in SEQ ID NO: 3.

In one example, the present disclosure provides a multicistronic self-replicating RNA, wherein the RNA comprises, in order from 5′ to 3′:
a) a first nucleotide sequence encoding a N1 neuraminidase operably linked to a minimal SG promoter encoded by a sequence set forth in SEQ ID NO: 1; and
b) a second nucleotide sequence encoding a H5 hemagglutinin protein operably linked to an IRES encoded by a sequence set forth in SEQ ID NO: 4.

In one example, the antigens are an influenza A virus hemagglutinin (HA) protein and a matrix (M) protein. For example, the antigens are a H5 hemagglutinin protein and/or a M1 matrix protein. In one example, the M1 neuraminidase is an A/Puerto Rico/8/1934 (PR8-X) strain. In another example, the M1 neuraminidase is an A/California/07/09 strain. In one example, the antigens are a H5 hemagglutinin subtype influenza A virus strain encoded by a sequence set forth in SEQ ID NO: 5 and a M1 matrix protein subtype influenza A virus strain encoded by a sequence set forth in SEQ ID NO: 16 or SEQ ID NO: 29.

In one example, the present disclosure provides a polynucleotide, wherein the polynucleotide comprises, in order from 5′ to 3′:
a) a first nucleotide sequence encoding a H5 hemagglutinin protein; and a second nucleotide sequence encoding a M1 matrix protein; or
b) a first nucleotide sequence encoding a M1 matrix protein; and a second nucleotide sequence encoding a H5 hemagglutinin protein.

In one example, the present disclosure provides a polynucleotide, wherein the polynucleotide comprises, in order from 5′ to 3′, a first nucleotide sequence encoding a H5 hemagglutinin protein; and a second nucleotide sequence encoding a M1 matrix protein.

In one example, the present disclosure provides a polynucleotide, wherein the polynucleotide comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; and a second nucleotide sequence encoding a M1 matrix protein operably linked to a regulatory element selected from the group consisting of a SG promoter and an IRES.

In one example, the present disclosure provides a RNA, wherein the RNA comprises, in order from 5′ to 3′:
a) a first nucleotide sequence encoding a H5 hemagglutinin protein; and a second nucleotide sequence encoding a M1 matrix protein; or
b) a first nucleotide sequence encoding a M1 matrix protein; and a second nucleotide sequence encoding a H5 hemagglutinin protein.

In one example, the present disclosure provides a RNA, wherein the RNA comprises, in order from 5′ to 3′, a first nucleotide sequence encoding a H5 hemagglutinin protein; and a second nucleotide sequence encoding a M1 matrix protein.

In one example, the present disclosure provides a RNA, wherein the RNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; and a second nucleotide sequence encoding a M1 matrix protein operably linked to a regulatory element selected from the group consisting of a SG promoter and an IRES.

In one example, the present disclosure provides a cRNA, wherein the cRNA comprises, in order from 5′ to 3′:
a) a first nucleotide sequence encoding a H5 hemagglutinin protein; and a second nucleotide sequence encoding a M1 matrix protein; or
b) a first nucleotide sequence encoding a M1 matrix protein; and a second nucleotide sequence encoding a H5 hemagglutinin protein.

In one example, the present disclosure provides a cRNA, wherein the cRNA comprises, in order from 5′ to 3′, a first nucleotide sequence encoding a H5 hemagglutinin protein; and a second nucleotide sequence encoding a M1 matrix protein.

In one example, the present disclosure provides a cRNA, wherein the cRNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; and a second nucleotide sequence encoding a M1 matrix protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein; and a second nucleotide sequence encoding a M1 matrix protein; or
b) a first nucleotide sequence encoding a M1 matrix protein; and a second nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a H5 hemagglutinin protein; and a second nucleotide sequence encoding a M1 matrix protein.

In one example, the present disclosure provides a multicistronic self-replicating RNA, wherein the RNA comprises, in order from 5′ to 3′, a first nucleotide sequence encoding a H5 hemagglutinin protein operably linked to a SG promoter; and a second nucleotide sequence encoding a M1 matrix protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1; and
b) a second nucleotide sequence encoding a M1 matrix protein operably linked to a minimal SG promoter encoded by a sequence set forth in SEQ ID NO: 1.

In one example, the antigens are an influenza A virus hemagglutinin (HA) protein, a neuraminidase (NA) protein and a matrix (M) protein. For example, the antigens are a H5 hemagglutinin protein and/or a N1 neuraminidase protein and/or a M1 matrix protein. In one example, the antigens are a H5 hemagglutinin subtype influenza A virus strain encoded by a sequence set forth in SEQ ID NO: 5, a N1 neuraminidase subtype influenza A virus strain encoded by a sequence set forth in SEQ ID NO: 6 and a M1 matrix protein subtype influenza A virus strain encoded by a sequence set forth in SEQ ID NO: 16 or SEQ ID NO: 29.

In one example, the present disclosure provides a polynucleotide, wherein the polynucleotide comprises, in order from 5′ to 3′:
a) a first nucleotide sequence encoding a H5 hemagglutinin protein; a second nucleotide sequence encoding a N1 neuraminidase protein; and a third nucleotide sequence encoding a M1 matrix protein; or
b) a first nucleotide sequence encoding a M1 matrix protein, a second nucleotide sequence encoding a N1 neuraminidase protein; and a third nucleotide sequence encoding a H5 hemagglutinin protein.

In one example, the present disclosure provides a polynucleotide, wherein the polynucleotide comprises, in order from 5′ to 3′, a first nucleotide sequence encoding a H5 hemagglutinin protein; a second nucleotide sequence encoding a N1 neuraminidase protein and a third nucleotide sequence encoding a M1 matrix protein.

In one example, the present disclosure provides a polynucleotide, wherein the polynucleotide comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; a second nucleotide sequence encoding a N1 neuraminidase protein operably linked to a regulatory element selected from the group consisting of a SG promoter and an IRES; and a third nucleotide sequence encoding a M1 matrix protein operably linked to a regulatory element selected from the group consisting of a SG promoter and an IRES.

In one example, the present disclosure provides a polynucleotide, wherein the polynucleotide comprises, in order from 5′ to 3′, a first nucleotide sequence encoding a M1 matrix protein; a second nucleotide sequence encoding a N1 neuraminidase protein and a third nucleotide sequence encoding a H5 hemagglutinin protein.

In one example, the present disclosure provides a polynucleotide, wherein the polynucleotide comprises, in order from 5′ to 3′, a first nucleotide sequence encoding a M1 matrix protein operably linked to a regulatory element selected from the group consisting of a Kozak consensus sequence, an IRES, a SG promoter and combinations thereof; a second nucleotide sequence encoding a N1 neuraminidase protein operably linked to a regulatory element selected from the group consisting of a SG promoter and an IRES; and a third nucleotide sequence encoding a H5 hemagglutinin protein operably linked to a regulatory element selected from the group consisting of a SG promoter and an IRES.

In one example, the present disclosure provides a RNA, wherein the RNA comprises, in order from 5′ to 3′:
a) a first nucleotide sequence encoding a H5 hemagglutinin protein; a second nucleotide sequence encoding a N1 neuraminidase protein; and a third nucleotide sequence encoding a M1 matrix protein; or
b) a first nucleotide sequence encoding a M1 matrix protein, a second nucleotide sequence encoding a N1 neuraminidase protein; and a third nucleotide sequence encoding a H5 hemagglutinin protein.

In one example, the present disclosure provides a RNA, wherein the RNA comprises, in order from 5′ to 3′, a first nucleotide sequence encoding a H5 hemagglutinin protein; a second nucleotide sequence encoding a N1 neuraminidase protein and a third nucleotide sequence encoding a M1 matrix protein.

In one example, the present disclosure provides a RNA, wherein the RNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; a second nucleotide sequence encoding a N1 neuraminidase protein operably linked to a regulatory element selected from the group consisting of a SG promoter and an IRES; and a third nucleotide sequence encoding a M1 matrix protein operably linked to a regulatory element selected from the group consisting of a SG promoter and an IRES.

In one example, the present disclosure provides a RNA, wherein the RNA comprises, in order from 5′ to 3′, a first nucleotide sequence encoding a M1 matrix protein; a second nucleotide sequence encoding a N1 neuraminidase protein and a third nucleotide sequence encoding a H5 hemagglutinin protein.

In one example, the present disclosure provides a RNA, wherein the RNA comprises, in order from 5′ to 3′, a first nucleotide sequence encoding a M1 matrix protein operably linked to a regulatory element selected from the group consisting of a Kozak consensus sequence, an IRES, a SG promoter and combinations thereof; a second nucleotide sequence encoding a N1 neuraminidase protein operably linked to a regulatory element selected from the group consisting of a SG promoter and an IRES; and a third nucleotide sequence encoding a H5 hemagglutinin protein operably linked to a regulatory element selected from the group consisting of a SG promoter and an IRES.

In one example, the present disclosure provides a cRNA, wherein the cRNA comprises, in order from 5′ to 3′:
a) a first nucleotide sequence encoding a H5 hemagglutinin protein; a second nucleotide sequence encoding a N1 neuraminidase protein; and a third nucleotide sequence encoding a M1 matrix protein; or
b) a first nucleotide sequence encoding a M1 matrix protein, a second nucleotide sequence encoding a N1 neuraminidase protein; and a third nucleotide sequence encoding a H5 hemagglutinin protein.

In one example, the present disclosure provides a cRNA, wherein the cRNA comprises, in order from 5′ to 3′, a first nucleotide sequence encoding a H5 hemagglutinin protein; a second nucleotide sequence encoding a N1 neuraminidase protein and a third nucleotide sequence encoding a M1 matrix protein.

In one example, the present disclosure provides a cRNA, wherein the cRNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; a second nucleotide sequence encoding a N1 neuraminidase protein operably linked to a regulatory element selected from the group consisting of a SG promoter and an IRES; and a third nucleotide sequence encoding a M1 matrix protein operably linked to a regulatory element selected from the group consisting of a SG promoter and an IRES.

In one example, the present disclosure provides a cRNA, wherein the RNA comprises, in order from 5′ to 3′, a first nucleotide sequence encoding a M1 matrix protein; a second nucleotide sequence encoding a N1 neuraminidase protein and a third nucleotide sequence encoding a H5 hemagglutinin protein.

In one example, the present disclosure provides a cRNA, wherein the RNA comprises, in order from 5′ to 3′, a first nucleotide sequence encoding a M1 matrix protein operably linked to a regulatory element selected from the group consisting of a Kozak consensus sequence, an IRES, a SG promoter and combinations thereof; a second nucleotide sequence encoding a N1 neuraminidase protein operably linked to a regulatory element selected from the group consisting of a SG promoter and an IRES; and a third nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein; a second nucleotide sequence encoding a N1 neuraminidase protein; and a third nucleotide sequence encoding a M1 matrix protein; or
b) a first nucleotide sequence encoding a M1 matrix protein, a second nucleotide sequence encoding a N1 neuraminidase protein; and a third nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a H5 hemagglutinin protein; a second nucleotide sequence encoding a N1 neuraminidase protein and a third nucleotide sequence encoding a M1 matrix protein.

In one example, the present disclosure provides a multicistronic self-replicating RNA, wherein the RNA comprises, in order from 5′ to 3′, a first nucleotide sequence encoding a H5 hemagglutinin protein operably linked to a SG promoter; a second nucleotide sequence encoding a N1 neuraminidase protein operably linked to a regulatory element selected from the group consisting of a SG promoter and an IRES; and a third nucleotide sequence encoding a M1 matrix protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1;
b) a second nucleotide sequence encoding a N1 neuraminidase protein operably linked to a minimal SG promoter encoded by a sequence set forth in SEQ ID NO: 1; and
c) a third nucleotide sequence encoding a M1 matrix protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′, a first nucleotide sequence encoding a M1 matrix protein; a second nucleotide sequence encoding a N1 neuraminidase protein and a third nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a M1 matrix protein operably linked to a SG promoter; a second nucleotide sequence encoding a N1 neuraminidase protein operably linked to a regulatory element selected from the group consisting of a SG promoter and an IRES; and a third nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a M1 matrix protein operably linked to a minimal SG promoter encoded by a sequence set forth in SEQ ID NO: 1;
b) a second nucleotide sequence encoding a N1 neuraminidase protein operably linked to a minimal SG promoter encoded by a sequence set forth in SEQ ID NO: 1; and
c) a third nucleotide sequence encoding a H5 hemagglutinin protein operably linked to a minimal SG promoter encoded by a sequence set forth in SEQ ID NO: 1.

In one example, the antigens are an influenza A virus HA protein, a NA protein and a M protein. For example, the antigens are a H5 hemagglutinin protein and/or a N1 neuraminidase protein and/or a M1 matrix protein and/or a M2 matrix protein. In one example, an antigens are a H5 hemagglutinin subtype influenza A virus strain encoded by a sequence set forth in SEQ ID NO: 5, a N1 neuraminidase subtype influenza A virus strain encoded by a sequence set forth in SEQ ID NO: 6, a M1 matrix protein subtype influenza A virus strain encoded by a sequence set forth in SEQ ID NO: 16 or SEQ ID NO: 29 and a M2 matrix protein subtype influenza A virus strain encoded by a sequence set forth in SEQ ID NO: 17.

In one example, the present disclosure provides a polynucleotide, wherein the polynucleotide comprises, in order from 5′ to 3′, a first nucleotide sequence encoding a M1 matrix protein; a second nucleotide sequence encoding a M2 matrix protein; a third nucleotide sequence encoding a N1 neuraminidase protein and a fourth nucleotide sequence encoding a H5 hemagglutinin protein.

In one example, the present disclosure provides a polynucleotide, wherein the polynucleotide comprises, in order from 5′ to 3′, a first nucleotide sequence encoding a M1 matrix protein operably linked to a regulatory element selected from the group consisting of a Kozak consensus sequence, an IRES, a SG promoter and combinations thereof; a second nucleotide sequence encoding a M2 matrix protein operably linked to a regulatory element selected from the group consisting of a SG promoter and an IRES; a third nucleotide sequence encoding a N1 neuraminidase protein operably linked to a regulatory element selected from the group consisting of a SG promoter and an IRES; and a fourth nucleotide sequence encoding a H5 hemagglutinin protein operably linked to a regulatory element selected from the group consisting of a SG promoter and an IRES.

In one example, the present disclosure provides a RNA, wherein the RNA comprises, in order from 5′ to 3′, a first nucleotide sequence encoding a M1 matrix protein; a second nucleotide sequence encoding a M2 matrix protein; a third nucleotide sequence encoding a N1 neuraminidase protein and a fourth nucleotide sequence encoding a H5 hemagglutinin protein.

In one example, the present disclosure provides a RNA, wherein the RNA comprises, in order from 5′ to 3′, a first nucleotide sequence encoding a M1 matrix protein operably linked to a regulatory element selected from the group consisting of a Kozak consensus sequence, an IRES, a SG promoter and combinations thereof; a second nucleotide sequence encoding a M2 matrix protein operably linked to a regulatory element selected from the group consisting of a SG promoter and an IRES; a third nucleotide sequence encoding a N1 neuraminidase protein operably linked to a regulatory element selected from the group consisting of a SG promoter and an IRES; and a fourth nucleotide sequence encoding a H5 hemagglutinin protein operably linked to a regulatory element selected from the group consisting of a SG promoter and an IRES.

In one example, the present disclosure provides a cRNA, wherein the RNA comprises, in order from 5′ to 3′, a first nucleotide sequence encoding a M1 matrix protein; a second nucleotide sequence encoding a M2 matrix protein; a third nucleotide sequence encoding a N1 neuraminidase protein and a fourth nucleotide sequence encoding a H5 hemagglutinin protein.

In one example, the present disclosure provides a cRNA, wherein the RNA comprises, in order from 5′ to 3′, a first nucleotide sequence encoding a M1 matrix protein operably linked to a regulatory element selected from the group consisting of a Kozak consensus sequence, an IRES, a SG promoter and combinations thereof; a second nucleotide sequence encoding a M2 matrix protein operably linked to a regulatory element selected from the group consisting of a SG promoter and an IRES; a third nucleotide sequence encoding a N1 neuraminidase protein operably linked to a regulatory element selected from the group consisting of a SG promoter and an IRES; and a fourth nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′, a first nucleotide sequence encoding a M1 matrix protein; a second nucleotide sequence encoding a M2 matrix protein; a third nucleotide sequence encoding a N1 neuraminidase protein and a fourth nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a M1 matrix protein operably linked to a SG promoter; a second nucleotide sequence encoding a M2 matrix protein operably linked to a regulatory element selected from the group consisting of a SG promoter and an IRES; a third nucleotide sequence encoding a N1 neuraminidase protein operably linked to a regulatory element selected from the group consisting of a SG promoter and an IRES; and a fourth nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a M1 matrix protein operably linked to a minimal SG promoter encoded by a sequence set forth in SEQ ID NO: 1;
b) a second nucleotide sequence encoding a M2 matrix protein operably linked to a minimal SG promoter encoded by a sequence set forth in SEQ ID NO: 1
c) a third nucleotide sequence encoding a N1 neuraminidase protein operably linked to a minimal SG promoter encoded by a sequence set forth in SEQ ID NO: 1; and
d) a fourth nucleotide sequence encoding a H5 hemagglutinin protein operably linked to a minimal SG promoter encoded by a sequence set forth in SEQ ID NO: 1.

In one example, the antigens are an influenza A virus HA protein and a NS protein. For example, the antigens are a H5 hemagglutinin protein and/or a NS1 non-structural protein. In one example, the antigens are a H5 hemagglutinin subtype influenza A virus strain encoded by a sequence set forth in SEQ ID NO: 5 and a NS1 non-structural protein subtype influenza A virus strain encoded by a sequence set forth in SEQ ID NO: 18.

In one example, the present disclosure provides a polynucleotide, wherein the polynucleotide comprises, in order from 5′ to 3′:
a) a first nucleotide sequence encoding a H5 hemagglutinin protein; and a second nucleotide sequence encoding a NS1 non-structural protein; or
b) a first nucleotide sequence encoding a NS1 non-structural protein; and a second nucleotide sequence encoding a H5 hemagglutinin protein.

In one example, the present disclosure provides a polynucleotide, wherein the polynucleotide comprises, in order from 5′ to 3′, a first nucleotide sequence encoding a H5 hemagglutinin protein; and a second nucleotide sequence encoding a NS1 non-structural protein.

In one example, the present disclosure provides a polynucleotide, wherein the polynucleotide comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; a second nucleotide sequence encoding a NS1 non-structural protein operably linked to a regulatory element selected from the group consisting of a SG promoter and an IRES.

In one example, the present disclosure provides a polynucleotide, wherein the polynucleotide comprises, in order from 5′ to 3′, a first nucleotide sequence encoding a NS1 non-structural protein; and a second nucleotide sequence encoding a H5 hemagglutinin protein.

In one example, the present disclosure provides a polynucleotide, wherein the polynucleotide comprises, in order from 5′ to 3′, a first nucleotide sequence encoding a NS1 non-structural protein operably linked to a regulatory element selected from the group consisting of a Kozak consensus sequence, an IRES, a SG promoter and combinations thereof; a second nucleotide sequence encoding a H5 hemagglutinin protein operably linked to a regulatory element selected from the group consisting of a SG promoter and an IRES.

In one example, the present disclosure provides a RNA, wherein the RNA comprises, in order from 5′ to 3′:
a) a first nucleotide sequence encoding a H5 hemagglutinin protein; and a second nucleotide sequence encoding a NS1 non-structural protein; or
b) a first nucleotide sequence encoding a NS1 non-structural protein; and a second nucleotide sequence encoding a H5 hemagglutinin protein.

In one example, the present disclosure provides a RNA, wherein the RNA comprises, in order from 5′ to 3′, a first nucleotide sequence encoding a H5 hemagglutinin protein; and a second nucleotide sequence encoding a NS1 non-structural protein.

In one example, the present disclosure provides a RNA, wherein the RNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; a second nucleotide sequence encoding a NS1 non-structural protein operably linked to a regulatory element selected from the group consisting of a SG promoter and an IRES.

In one example, the present disclosure provides a RNA, wherein the RNA comprises, in order from 5′ to 3′, a first nucleotide sequence encoding a NS1 non-structural protein; and a second nucleotide sequence encoding a H5 hemagglutinin protein.

In one example, the present disclosure provides a RNA, wherein the RNA comprises, in order from 5′ to 3′, a first nucleotide sequence encoding a NS1 non-structural protein operably linked to a regulatory element selected from the group consisting of a Kozak consensus sequence, an IRES, a SG promoter and combinations thereof; a second nucleotide sequence encoding a H5 hemagglutinin protein operably linked to a regulatory element selected from the group consisting of a SG promoter and an IRES.

In one example, the present disclosure provides a cRNA, wherein the cRNA comprises, in order from 5′ to 3′:
a) a first nucleotide sequence encoding a H5 hemagglutinin protein; and a second nucleotide sequence encoding a NS1 non-structural protein; or
b) a first nucleotide sequence encoding a NS1 non-structural protein; and a second nucleotide sequence encoding a H5 hemagglutinin protein.

In one example, the present disclosure provides a cRNA, wherein the cRNA comprises, in order from 5′ to 3′, a first nucleotide sequence encoding a H5 hemagglutinin protein; and a second nucleotide sequence encoding a NS1 non-structural protein.

In one example, the present disclosure provides a cRNA, wherein the cRNA comprises, in order from 5′ to 3′, a first nucleotide sequence encoding a H5 hemagglutinin protein operably linked to a regulatory element selected from the group consisting of a Kozak consensus sequence, an IRES, a SG promoter and combinations thereof; a second nucleotide sequence encoding a NS1 non-structural protein operably linked to a regulatory element selected from the group consisting of a SG promoter and an IRES.

In one example, the present disclosure provides a cRNA, wherein the cRNA comprises, in order from 5′ to 3′, a first nucleotide sequence encoding a NS1 non-structural protein; and a second nucleotide sequence encoding a H5 hemagglutinin protein.

In one example, the present disclosure provides a cRNA, wherein the cRNA comprises, in order from 5′ to 3′, a first nucleotide sequence encoding a NS1 non-structural protein operably linked to a regulatory element selected from the group consisting of a Kozak consensus sequence, an IRES, a SG promoter and combinations thereof; a second nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein; and a second nucleotide sequence encoding a NS1 non-structural protein; or
b) a first nucleotide sequence encoding a NS1 non-structural protein; and a second nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a H5 hemagglutinin protein; and a second nucleotide sequence encoding a NS1 non-structural protein.

In one example, the present disclosure provides a multicistronic self-replicating RNA, wherein the RNA comprises, in order from 5′ to 3′, a first nucleotide sequence encoding a H5 hemagglutinin protein operably linked to a SG promoter; a second nucleotide sequence encoding a NS1 non-structural protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a H5 hemagglutinin protein operably linked to a minimal SG promoter encoded by a sequence set forth in SEQ ID NO: 1; and
b) a second nucleotide sequence encoding a NS1 non-structural protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′, a first nucleotide sequence encoding a NS1 non-structural protein; and a second nucleotide sequence encoding a H5 hemagglutinin protein.

In one example, the present disclosure provides a multicistronic self-replicating RNA, wherein the RNA comprises, in order from 5′ to 3′, a first nucleotide sequence encoding a NS1 non-structural protein operably linked to a SG promoter; a second nucleotide sequence encoding a H5 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a NS1 non-structural protein operably linked to a minimal SG promoter encoded by a sequence set forth in SEQ ID NO: 1; and
b) a second nucleotide sequence encoding a H5 hemagglutinin protein operably linked to a minimal SG promoter encoded by a sequence set forth in SEQ ID NO: 1.

In one example, the antigens are an influenza A virus M protein and a NP. For example, the antigens are a M1 matrix protein and/or a NP protein. In one example, the NP protein is an A/California/07/09 strain. In one example, the antigens are a M1 matrix protein subtype influenza A virus strain encoded by a sequence set forth in SEQ ID NO: 16 or SEQ ID NO: 29 and a NP nucleoprotein encoded by a sequence set forth in SEQ ID NO: 28.

In one example, the present disclosure provides a polynucleotide, wherein the polynucleotide comprises, in order from 5′ to 3′:
a) a first nucleotide sequence encoding a M1 matrix protein; and a second nucleotide sequence encoding a NP nucleoprotein; or
b) a first nucleotide sequence encoding a NP nucleoprotein; and a second nucleotide sequence encoding a M1 matrix protein.

In one example, the present disclosure provides a polynucleotide, wherein the polynucleotide comprises, in order from 5′ to 3′, a first nucleotide sequence encoding a M1 matrix protein; and a second nucleotide sequence encoding a NP nucleoprotein.

In one example, the present disclosure provides a polynucleotide, wherein the polynucleotide comprises, in order from 5′ to 3′, a first nucleotide sequence encoding M1 matrix protein operably linked to a regulatory element selected from the group consisting of a Kozak consensus sequence, an IRES, a SG promoter and combinations thereof; a second nucleotide sequence encoding a NP nucleoprotein operably linked to a regulatory element selected from the group consisting of a SG promoter and an IRES.

In one example, the present disclosure provides a RNA, wherein the RNA comprises, in order from 5′ to 3′:
a) a first nucleotide sequence encoding a M1 matrix protein; and a second nucleotide sequence encoding a NP nucleoprotein; or
b) a first nucleotide sequence encoding a NP nucleoprotein; and a second nucleotide sequence encoding a M1 matrix protein.

In one example, the present disclosure provides a RNA, wherein the RNA comprises, in order from 5′ to 3′, a first nucleotide sequence encoding a M1 matrix protein; and a second nucleotide sequence encoding a NP nucleoprotein.

In one example, the present disclosure provides a RNA, wherein the RNA comprises, in order from 5′ to 3′, a first nucleotide sequence encoding M1 matrix protein operably linked to a regulatory element selected from the group consisting of a Kozak consensus sequence, an IRES, a SG promoter and combinations thereof; a second nucleotide sequence encoding a NP nucleoprotein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a M1 matrix protein; and a second nucleotide sequence encoding a NP nucleoprotein; or
b) a first nucleotide sequence encoding a NP nucleoprotein; and a second nucleotide sequence encoding a M1 matrix protein.

In one example, the present disclosure provides a multicistronic self-replicating RNA, wherein the RNA comprises, in order from 5′ to 3′, a first nucleotide sequence encoding a M1 matrix protein; and a second nucleotide sequence encoding a NP nucleoprotein.

In one example, the present disclosure provides a multicistronic self-replicating RNA, wherein the RNA comprises, in order from 5′ to 3′, a first nucleotide sequence encoding M1 matrix protein operably linked to a SG promoter; a second nucleotide sequence encoding a NP nucleoprotein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a M1 matrix protein operably linked to a minimal SG promoter encoded by a sequence set forth in SEQ ID NO: 1; and
b) a second nucleotide sequence encoding a NP nucleoprotein operably linked to a minimal SG promoter encoded by a sequence set forth in SEQ ID NO: 1.

In one example, the present disclosure provides a multicistronic self-replicating RNA, wherein the RNA comprises, in order from 5′ to 3′:
a) a first nucleotide sequence encoding a M1 matrix protein operably linked to a minimal SG promoter encoded by a sequence set forth in SEQ ID NO: 1; and
b) a second nucleotide sequence encoding a NP nucleoprotein operably linked to an extended SG promoter encoded by a sequence set forth in SEQ ID NO: 2.

In one example, the present disclosure provides a polynucleotide, wherein the polynucleotide comprises, in order from 5′ to 3′:
c) a first nucleotide sequence encoding a H3 hemagglutinin protein; and a second nucleotide sequence encoding a N2 neuraminidase protein; or
d) a first nucleotide sequence encoding a N2 neuraminidase protein; and a second nucleotide sequence encoding a H3 hemagglutinin protein.

In one example, the present disclosure provides a polynucleotide, wherein the polynucleotide comprises, in order from 5′ to 3′, a first nucleotide sequence encoding a H3 hemagglutinin protein; and a second nucleotide sequence encoding a N2 neuraminidase protein.

In one example, the present disclosure provides a polynucleotide, wherein the polynucleotide comprises, in order from 5′ to 3′, a first nucleotide sequence encoding a H3 hemagglutinin protein operably linked to a regulatory element selected from the group consisting of a Kozak consensus sequence, an IRES, a SG promoter and combinations thereof; a second nucleotide sequence encoding a N2 neuraminidase protein operably linked to a regulatory element selected from the group consisting of a SG promoter and an IRES.

In one example, the present disclosure provides a polynucleotide, wherein the polynucleotide comprises, in order from 5′ to 3′, a first nucleotide sequence encoding a N2 neuraminidase protein; and a second nucleotide sequence encoding a H3 hemagglutinin protein.

In one example, the present disclosure provides a polynucleotide, wherein the polynucleotide comprises, in order from 5′ to 3′, a first nucleotide sequence encoding a N2 neuraminidase protein operably linked to a regulatory element selected from the group consisting of a Kozak consensus sequence, an IRES, a SG promoter and combinations thereof; a second nucleotide sequence encoding a H3 hemagglutinin protein operably linked to a regulatory element selected from the group consisting of a SG promoter and an IRES.

In one example, the present disclosure provides a RNA, wherein the RNA comprises, in order from 5′ to 3′:
c) a first nucleotide sequence encoding a H3 hemagglutinin protein; and a second nucleotide sequence encoding a N2 neuraminidase protein; or
d) a first nucleotide sequence encoding a N2 neuraminidase protein; and a second nucleotide sequence encoding a H3 hemagglutinin protein.

In one example, the present disclosure provides a RNA, wherein the RNA comprises, in order from 5′ to 3′, a first nucleotide sequence encoding a H3 hemagglutinin protein; and a second nucleotide sequence encoding a N2 neuraminidase protein.

In one example, the present disclosure provides a RNA, wherein the RNA comprises, in order from 5′ to 3′, a first nucleotide sequence encoding a H3 hemagglutinin protein operably linked to a regulatory element selected from the group consisting of a Kozak consensus sequence, an IRES, a SG promoter and combinations thereof; a second nucleotide sequence encoding a N2 neuraminidase protein operably linked to a regulatory element selected from the group consisting of a SG promoter and an IRES.

In one example, the present disclosure provides a RNA, wherein the RNA comprises, in order from 5′ to 3′, a first nucleotide sequence encoding a N2 neuraminidase protein; and a second nucleotide sequence encoding a H3 hemagglutinin protein.

In one example, the present disclosure provides a RNA, wherein the RNA comprises, in order from 5′ to 3′, a first nucleotide sequence encoding a N2 neuraminidase protein operably linked to a regulatory element selected from the group consisting of a Kozak consensus sequence, an IRES, a SG promoter and combinations thereof; a second nucleotide sequence encoding a H3 hemagglutinin protein operably linked to a regulatory element selected from the group consisting of a SG promoter and an IRES.

In one example, the present disclosure provides a cRNA, wherein the cRNA comprises, in order from 5′ to 3′:
c) a first nucleotide sequence encoding a H3 hemagglutinin protein; and a second nucleotide sequence encoding a N2 neuraminidase protein; or
d) a first nucleotide sequence encoding a N2 neuraminidase protein; and a second nucleotide sequence encoding a H3 hemagglutinin protein.

In one example, the present disclosure provides a cRNA, wherein the cRNA comprises, in order from 5′ to 3′, a first nucleotide sequence encoding a H3 hemagglutinin protein; and a second nucleotide sequence encoding a N2 neuraminidase protein.

In one example, the present disclosure provides a cRNA, wherein the cRNA comprises, in order from 5′ to 3′, a first nucleotide sequence encoding a H3 hemagglutinin protein operably linked to a regulatory element selected from the group consisting of a Kozak consensus sequence, an IRES, a SG promoter and combinations thereof; a second nucleotide sequence encoding a N2 neuraminidase protein operably linked to a regulatory element selected from the group consisting of a SG promoter and an IRES.

In one example, the present disclosure provides a cRNA, wherein the cRNA comprises, in order from 5′ to 3′, a first nucleotide sequence encoding a N2 neuraminidase protein; and a second nucleotide sequence encoding a H3 hemagglutinin protein.

In one example, the present disclosure provides a cRNA, wherein the cRNA comprises, in order from 5′ to 3′, a first nucleotide sequence encoding a N2 neuraminidase protein operably linked to a regulatory element selected from the group consisting of a Kozak consensus sequence, an IRES, a SG promoter and combinations thereof; a second nucleotide sequence encoding a H3 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
c) a first nucleotide sequence encoding a H3 hemagglutinin protein; and a second nucleotide sequence encoding a N2 neuraminidase protein; or
d) a first nucleotide sequence encoding a N2 neuraminidase protein; and a second nucleotide sequence encoding a H3 hemagglutinin protein.

In one example, the present disclosure provides a multicistronic self-replicating RNA, wherein the RNA comprises, in order from 5′ to 3′, a first nucleotide sequence encoding a H3 hemagglutinin protein; and a second nucleotide sequence encoding a N2 neuraminidase protein.

In one example, the present disclosure provides a multicistronic self-replicating RNA, wherein the RNA comprises, in order from 5′ to 3′, a first nucleotide sequence encoding a H3 hemagglutinin protein operably linked to a SG promoter; a second nucleotide sequence encoding a N2 neuraminidase protein operably linked to a regulatory element selected from the group consisting of a SG promoter and an IRES.

In one example, the present disclosure provides a multicistronic self-replicating RNA, wherein the RNA comprises, in order from 5′ to 3′:
c) a first nucleotide sequence encoding a H3 hemagglutinin protein operably linked to a minimal SG promoter encoded by a sequence set forth in SEQ ID NO: 1; and
d) a second nucleotide sequence encoding a N2 neuraminidase protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′, a first nucleotide sequence encoding a N2 neuraminidase protein; and a second nucleotide sequence encoding a H3 hemagglutinin protein.

In one example, the present disclosure provides a multicistronic self-replicating RNA, wherein the RNA comprises, in order from 5′ to 3′, a first nucleotide sequence encoding a N2 neuraminidase protein operably linked to a SG promoter; a second nucleotide sequence encoding a H3 hemagglutinin protein operably linked to a regulatory element selected from the group consisting of a SG promoter and an IRES.

In one example, the present disclosure provides a multicistronic self-replicating RNA, wherein the RNA comprises, in order from 5′ to 3′:
c) a first nucleotide sequence encoding a N2 neuraminidase protein operably linked to a minimal SG promoter encoded by a sequence set forth in SEQ ID NO: 1; and
d) a second nucleotide sequence encoding a H3 hemagglutinin protein operably linked to a minimal SG promoter encoded by a sequence set forth in SEQ ID NO: 1.

In one example, the antigens are an influenza B virus strain. The skilled person will be aware that influenza B viruses are not divided into subtypes but are classified into two lineages, namely, B/Yamagata and B/Victoria.

In one example, the antigens are a B/Yamagata influenza B virus strain. For example, the influenza B virus strain is a B/Singapore/INFTT 16 0610/16 (By) virus strain. In another example, the antigens are a B/Victoria influenza B virus strain. In one example, the antigens are influenza B virus strains in the same lineage. In another example, the antigens are influenza B virus strains in different lineages.

In one example, the antigens are an influenza B virus Hyam protein and/or a Nyam protein. For example, antigens are an influenza B virus Hyam protein. In another example, the antigens are an influenza B virus Nyam protein. In a further example, the antigens are an influenza B virus Hyam and Nyam protein. In one example, the antigens are a Hyam subtype influenza B virus strain encoded by a sequence set forth in SEQ ID NO: 56 and a Nyam subtype influenza B virus strain encoded by a sequence set forth in SEQ ID NO: 57.

In one example, the present disclosure provides a polynucleotide, wherein the polynucleotide comprises, in order from 5′ to 3′:
e) a first nucleotide sequence encoding a Hyam protein; and a second nucleotide sequence encoding a Nyam protein; or
f) a first nucleotide sequence encoding a Nyam protein; and a second nucleotide sequence encoding a Hyam protein.

In one example, the present disclosure provides a polynucleotide, wherein the polynucleotide comprises, in order from 5′ to 3′, a first nucleotide sequence encoding a Hyam protein; and a second nucleotide sequence encoding a Nyam protein.

In one example, the present disclosure provides a polynucleotide, wherein the polynucleotide comprises, in order from 5′ to 3′, a first nucleotide sequence encoding a Hyam protein operably linked to a regulatory element selected from the group consisting of a Kozak consensus sequence, an IRES, a SG promoter and combinations thereof; a second nucleotide sequence encoding a Nyam protein operably linked to a regulatory element selected from the group consisting of a SG promoter and an IRES.

In one example, the present disclosure provides a polynucleotide, wherein the polynucleotide comprises, in order from 5′ to 3′, a first nucleotide sequence encoding a Nyam protein; and a second nucleotide sequence encoding a Hyam protein.

In one example, the present disclosure provides a polynucleotide, wherein the polynucleotide comprises, in order from 5′ to 3′, a first nucleotide sequence encoding a Nyam protein operably linked to a regulatory element selected from the group consisting of a Kozak consensus sequence, an IRES, a SG promoter and combinations thereof; a second nucleotide sequence encoding a Hyam protein operably linked to a regulatory element selected from the group consisting of a SG promoter and an IRES.

In one example, the present disclosure provides a RNA, wherein the RNA comprises, in order from 5′ to 3′:
e) a first nucleotide sequence encoding a Hyam protein; and a second nucleotide sequence encoding a Nyam protein; or
f) a first nucleotide sequence encoding a Nyam protein; and a second nucleotide sequence encoding a Hyam protein.

In one example, the present disclosure provides a RNA, wherein the RNA comprises, in order from 5′ to 3′, a first nucleotide sequence encoding a Hyam protein; and a second nucleotide sequence encoding a Nyam protein.

In one example, the present disclosure provides a RNA, wherein the RNA comprises, in order from 5′ to 3′, a first nucleotide sequence encoding a Hyam protein operably linked to a regulatory element selected from the group consisting of a Kozak consensus sequence, an IRES, a SG promoter and combinations thereof; a second nucleotide sequence encoding a Nyam protein operably linked to a regulatory element selected from the group consisting of a SG promoter and an IRES.

In one example, the present disclosure provides a RNA, wherein the RNA comprises, in order from 5′ to 3′, a first nucleotide sequence encoding a Nyam protein; and a second nucleotide sequence encoding a Hyam protein.

In one example, the present disclosure provides a RNA, wherein the RNA comprises, in order from 5′ to 3′, a first nucleotide sequence encoding a Nyam protein operably linked to a regulatory element selected from the group consisting of a Kozak consensus sequence, an IRES, a SG promoter and combinations thereof; a second nucleotide sequence encoding a Hyam protein operably linked to a regulatory element selected from the group consisting of a SG promoter and an IRES.

In one example, the present disclosure provides a cRNA, wherein the cRNA comprises, in order from 5′ to 3′:
e) a first nucleotide sequence encoding a Hyam protein; and a second nucleotide sequence encoding a Nyam protein; or
f) a first nucleotide sequence encoding a Nyam protein; and a second nucleotide sequence encoding a Hyam protein.

In one example, the present disclosure provides a cRNA, wherein the cRNA comprises, in order from 5′ to 3′, a first nucleotide sequence encoding a Hyam protein; and a second nucleotide sequence encoding a Nyam protein.

In one example, the present disclosure provides a cRNA, wherein the cRNA comprises, in order from 5′ to 3′, a first nucleotide sequence encoding a Hyam protein operably linked to a regulatory element selected from the group consisting of a Kozak consensus sequence, an IRES, a SG promoter and combinations thereof; a second nucleotide sequence encoding a Nyam protein operably linked to a regulatory element selected from the group consisting of a SG promoter and an IRES.

In one example, the present disclosure provides a cRNA, wherein the cRNA comprises, in order from 5′ to 3′, a first nucleotide sequence encoding a Nyam protein; and a second nucleotide sequence encoding a Hyam protein.

In one example, the present disclosure provides a cRNA, wherein the cRNA comprises, in order from 5′ to 3′, a first nucleotide sequence encoding a Nyam protein operably linked to a regulatory element selected from the group consisting of a Kozak consensus sequence, an IRES, a SG promoter and combinations thereof; a second nucleotide sequence encoding a Hyam protein operably linked to a regulatory element selected from the group consisting of a SG promoter and an IRES.

In one example, the present disclosure provides a multicistronic self-replicating RNA, wherein the RNA comprises, in order from 5′ to 3′:
e) a first nucleotide sequence encoding a Hyam protein; and a second nucleotide sequence encoding a Nyam protein; or
f) a first nucleotide sequence encoding a Nyam protein; and a second nucleotide sequence encoding a Hyam protein.

In one example, the present disclosure provides a multicistronic self-replicating RNA, wherein the RNA comprises, in order from 5′ to 3′, a first nucleotide sequence encoding a Hyam protein; and a second nucleotide sequence encoding a Nyam protein.

In one example, the present disclosure provides a multicistronic self-replicating RNA, wherein the RNA comprises, in order from 5′ to 3′, a first nucleotide sequence encoding a Hyam protein operably linked to a SG promoter; a second nucleotide sequence encoding a Nyam protein operably linked to a regulatory element selected from the group consisting of a SG promoter and an IRES.

In one example, the present disclosure provides a multicistronic self-replicating RNA, wherein the RNA comprises, in order from 5′ to 3′:
e) a first nucleotide sequence encoding a Hyam protein operably linked to a minimal SG promoter encoded by a sequence set forth in SEQ ID NO: 1; and
f) a second nucleotide sequence encoding a Nyam protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′, a first nucleotide sequence encoding a Nyam protein; and a second nucleotide sequence encoding a Hyam protein.

In one example, the present disclosure provides a multicistronic self-replicating RNA, wherein the RNA comprises, in order from 5′ to 3′, a first nucleotide sequence encoding a Nyam protein operably linked to a SG promoter; a second nucleotide sequence encoding a Hyam protein operably linked to a regulatory element selected from the group consisting of a SG promoter and an IRES.

In one example, the present disclosure provides a multicistronic self-replicating RNA, wherein the RNA comprises, in order from 5′ to 3′:
e) a first nucleotide sequence encoding a Nyam protein operably linked to a minimal SG promoter encoded by a sequence set forth in SEQ ID NO: 1; and
f) a second nucleotide sequence encoding a Hyam protein operably linked to a minimal SG promoter encoded by a sequence set forth in SEQ ID NO: 1.

In one example, the antigens are viral antigens from a coronavirus.

In one example, the antigens are an alphacoronavirus, a betacoronavirus, a gammacoronavirus and/or a deltacoronavirus strain.

In one example, the antigens are an alphacoronavirus. For example, an alphacoronavirus is selected from the group consisting of Alphacoronavirus 1, Human coronavirus 229E (HCoV 229E), Human coronavirus NL63 (HCoV NL63), Miniopterus bat coronavirus 1, Miniopterus bat coronavirus HKU8, Porcine epidemic diarrhea virus, Rhinolophus bat coronavirus HKU2 and Scotophilus bat coronavirus 512.

In one example, the antigens are a betacoronavirus. For example, a betacoronavirus is selected from the group consisting of Betacoronavirus 1 (Bovine Coronavirus, Human coronavirus OC43), Hedgehog coronavirus 1, Human coronavirus HKU1 (HCoV HKU1), Middle East respiratory syndrome-related coronavirus (MERS-CoV), Murine coronavirus, Pipistrellus bat coronavirus HKU5, Rousettus bat coronavirus HKU9, Severe acute respiratory syndrome-related coronavirus (SARS-CoV, SARS-CoV-2) and Tylonycteris bat coronavirus HKU4. In one example, antigens are derived from a betacoronavirus selected from the group consisting of Middle East respiratory syndrome-related coronavirus (MERS-CoV) and Severe acute respiratory syndrome-related coronavirus (SARS-CoV or SARS-CoV-2). For example, the antigens are from MERS-CoV. In another example, the antigens are from SARS-CoV. In a further example, the antigens are from SARS-CoV-2. For example, the coronavirus is SARS-CoV-2.

In one example, the antigens are a gammacoronavirus. For example, a gammacoronavirus is selected from the group consisting of an Avian coronavirus and Beluga whale coronavirus SW1.

In one example, the antigens are a deltacoronavirus. For example, a deltacoronavirus is selected from the group consisting of Bulbul coronavirus HKU11 and Porcine coronavirus HKU15.

In one example, the antigens are a spike (S) protein and/or a nucleocapsid (N) protein of a coronavirus. For example, the antigens are a SARS-CoV-2 N protein and/or a S protein. In one example, the antigens are a SARS-CoV-2 N protein and/or a S protein from SARS-CoV-2 strain 2019-nCoV/USA-WA1/2020.

In one example, the antigens are a SARS-CoV-2 N protein. For example, the antigens are a SARS-CoV-2 N protein and are encoded by a sequence set forth in SEQ ID NO: 7.

In another example, the antigens are a SARS-CoV-2 S protein. For example, the antigens are a SARS-CoV-2 spike protein and are encoded by a sequence set forth in SEQ ID NO: 8.

In another example, the S protein is a mutant S protein.

In one example, a mutant S protein comprises a mutation in the receptor binding domain. For example, the mutation is selected from the group consisting of S438F, N439K, N440K, L441I, K444R, V445A, V445I, G446V, G446S, N450K, L452R, L452P, L455F, K458N, N460T, D467V, 1468F, 1468T, 1468V, E4710, 1472V, A475V, G476S, S477G, S477I, S477N, S477R, T478I, P479L, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S and Y505H, Y508H. In one example, a mutant S protein comprises a mutation in the receptor binding domain selected from the group consisting of N439K, N439L, L452R, S477N, T478I, V483A and E484D.

In one example, a mutant S protein comprises a mutation in the receptor binding domain. For example, the mutation is selected from the group consisting of R346K, K417N, K417T, S438F, N439K, N440K, L441I, K444R, V445A, V445I, G446V, G446S, N450K, L452R, L452P, L455F, K458N, N460T, D467V, I468F, I468T, I468V, E4710, 1472V, A475V, G476S, S477G, S477I, S477N, S477R, T478I, T478K, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S, N501Y, Y505H and Y508H. In one example, a mutant S protein comprises a mutation in the receptor binding domain selected from the group consisting of R346K, K417N, K417T, N439K, N439L, L452R, S477N, T478I, V483A, E484D, E484K and N501Y.

In one example, a mutant S protein comprises a mutation selected from the group consisting of P337S, F338L, F338C, G339D, E340K, V341I, A344S, T345S, R346K, A348S, A348T, W353R, N354D, N354K, N354S, S359N, D364Y, V367F, S373L, V382L, P384L, P384S, T385A, T393P, V395I, F400C, R403K, R403S, D405V, R408I, Q414E, Q414K, Q414P, Q414R, T415S, K417R, K417N, I418V, Y421S, Y423C, Y423F, Y423S, D427Y, R509K, V510L, V511E, V512L, L518I, H5190, A520S, A520V, P521R, P521S, A522P, A522S and D614G.

In one example, a mutant S protein comprises a mutation selected from the group consisting of L18F, D80A, T951, Y144S, Y145N, D215G, P337S, F338L, F338C, G339D, E340K, V341I, A344S, T345S, R346K, A348S, A348T, W353R, N354D, N354K, N354S, S359N, D364Y, V367F, S373L, V382L, P384L, P384S, T385A, T393P, V395I, F400C, R403K, R403S, D405V, R408I, Q414E, Q414K, Q414P, Q414R, T415S, K417N, K417T, K417R, I418V, Y421S, Y423C, Y423F, Y423S, D427Y, S438F, N439K, N440K, L441I, K444R, V445A, V445I, G446V, G446S, N450K, L452R, L452P, L455F, K458N, N460T, D467V, I468F, I468T, I468V, E4710, 1472V, A475V, G476S, S477G, S477I, S477N, S477R, T478I, T478K, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S, N501Y, Y505H, Y508H, R509K, V510L, V511E, V512L, L518I, H5190, A520S, A520V, P521R, P521S, A522P, A522S, A570D, D614G, P680H, P681H, A701V, T716I and D950N.

In one example, the mutant S protein: (i) lacks a furin cleavage site at the S1/S2 boundary and comprises RRAR to QQAA mutations at residues corresponding to nucleotides 682-685 of SEQ ID NO: 37; and/or (ii) lacks a furin cleavage site at the S2′ site; and/or (iii) comprises D to G mutation at residue corresponding to nucleotide 614 of SEQ ID NO: 37; and/or (iv) comprises insertion of two proline residues between residues corresponding to nucleotides 986 and 987 of SEQ ID NO: 37.

In one example, the S protein lacks a furin cleavage site at the S1/S2 boundary and comprises RRAR to QQAA mutations at residues corresponding to nucleotides 682-685 of SEQ ID NO: 37. For example, the mutant S protein is encoded by a sequence set forth in SEQ ID NO: 9.

In one example, the S protein lacks a furin cleavage site at the S2′ site.

In one example, the S protein comprises D to G mutation at residue corresponding to nucleotide 614 of SEQ ID NO: 37. For example, the mutant S protein is encoded by a sequence set forth in SEQ ID NO: 36.

In one example, the S protein comprises insertion of two proline residues between residues corresponding to nucleotides 986 and 987 of SEQ ID NO: 37.

In one example, the S protein (i) lacks a furin cleavage site at the S1/S2 boundary and comprises RRAR to QQAA mutations at residues corresponding to nucleotides 682-685 of SEQ ID NO: 37; and (ii) lacks a furin cleavage site at the S2′ site. For example, the mutant S protein is encoded by a sequence set forth in SEQ ID NO: 34.

In one example, the S protein (i) lacks a furin cleavage site at the S1/S2 boundary and comprises RRAR to QQAA mutations at residues corresponding to nucleotides 682-685 of SEQ ID NO: 37; and (ii) comprises D to G mutation at residue corresponding to nucleotide 614 of SEQ ID NO: 37. For example, the mutant S protein is encoded by a sequence set forth in SEQ ID NO: 33.

In one example, the S protein (i) lacks a furin cleavage site at the S1/S2 boundary and comprises RRAR to QQAA mutations at residues corresponding to nucleotides 682-685 of SEQ ID NO: 37; and (ii) comprises insertion of two proline residues between residues corresponding to nucleotides 986 and 987 of SEQ ID NO: 37. For example, the mutant S protein is encoded by a sequence set forth in SEQ ID NO: 32.

In one example, the S protein (i) lacks a furin cleavage site at the S1/S2 boundary and comprises RRAR to QQAA mutations at residues corresponding to nucleotides 682-685 of SEQ ID NO: 37; and (ii) lacks a furin cleavage site at the S2′ site; and (iii) comprises D to G mutation at residue corresponding to nucleotide 614 of SEQ ID NO: 37. For example, the mutant S protein is encoded by a sequence set forth in SEQ ID NO: 35.

In one example, the S protein (i) lacks a furin cleavage site at the S1/S2 boundary and comprises RRAR to QQAA mutations at residues corresponding to nucleotides 682-685 of SEQ ID NO: 37; and (ii) lacks a furin cleavage site at the S2′ site; and (iii) comprises insertion of two proline residues between residues corresponding to nucleotides 986 and 987 of SEQ ID NO: 37.

In one example, the S protein (i) lacks a furin cleavage site at the S2′ site; and (ii) comprises D to G mutation at residue corresponding to nucleotide 614 of SEQ ID NO: 37.

In one example, the S protein (i) lacks a furin cleavage site at the S2′ site; and (ii) comprises insertion of two proline residues between residues corresponding to nucleotides 986 and 987 of SEQ ID NO: 37.

In one example, the S protein (i) lacks a furin cleavage site at the S2′ site; and (ii) comprises D to G mutation at residue corresponding to nucleotide 614 of SEQ ID NO: 37; and (iii) comprises insertion of two proline residues between residues corresponding to nucleotides 986 and 987 of SEQ ID NO: 37.

In one example, the S protein (i) comprises D to G mutation at residue corresponding to nucleotide 614 of SEQ ID NO: 37; and (ii) comprises insertion of two proline residues between residues corresponding to nucleotides 986 and 987 of SEQ ID NO: 37.

In one example, the S protein (i) lacks a furin cleavage site at the S1/S2 boundary and comprises RRAR to QQAA mutations at residues corresponding to nucleotides 682-685 of SEQ ID NO: 37; and (ii) lacks a furin cleavage site at the S2′ site; and (iii) comprises D to G mutation at residue corresponding to nucleotide 614 of SEQ ID NO: 37; and (iv) comprises insertion of two proline residues between residues corresponding to nucleotides 986 and 987 of SEQ ID NO: 37.

In one example, the mutant S protein comprises (i) a N to Y mutation at residue corresponding to nucleotide 501 of SEQ ID NO: 37; and/or (ii) deletion of two residues corresponding to nucleotides 69 and 70 of SEQ ID NO: 37; and/or (iii) P to H mutation at residue corresponding to nucleotide 681 of SEQ ID NO: 37.

In one example, the mutant S protein comprises a N to Y mutation at a residue corresponding to nucleotide 501 of SEQ ID NO: 37, and deletion of two residues corresponding to nucleotides 69 and 70 of SEQ ID NO: 37, and a P to H mutation at a residue corresponding to nucleotide 681 of SEQ ID NO: 37.

In one example, the mutant S protein comprises a P to H mutation at a residue corresponding to nucleotide 681 of SEQ ID NO: 37.

In one example, the mutant S protein comprises (i) a K to N mutation at a residue corresponding to nucleotide 417 of SEQ ID NO: 37; and/or (ii) E to K mutation at residue corresponding to nucleotide 484 of SEQ ID NO: 37; and/or (iii) a N to Y mutation at a residue corresponding to nucleotide 501 of SEQ ID NO: 37.

In one example, the mutant S protein comprises a K to N mutation at a residue corresponding to nucleotide 417 of SEQ ID NO: 37.

In one example, the mutant S protein comprises a E to K mutation at a residue corresponding to nucleotide 484 of SEQ ID NO: 37.

In one example, the mutant S protein comprises a K to N mutation at a residue corresponding to nucleotide 417 of SEQ ID NO: 37, and a E to K mutation at a residue corresponding to nucleotide 484 of SEQ ID NO: 37, and a N to Y mutation at residue corresponding to nucleotide 501 of SEQ ID NO: 37.

In one example, the mutant S protein comprises (i) a K to T mutation at a residue corresponding to nucleotide 417 of SEQ ID NO: 37; and/or (ii) a E to K mutation at a residue corresponding to nucleotide 484 of SEQ ID NO: 37; and/or (iii) a N to Y mutation at a residue corresponding to nucleotide 501 of SEQ ID NO: 37.

In one example, the mutant S protein comprises a K to T mutation at a residue corresponding to nucleotide 417 of SEQ ID NO: 37.

In one example, the mutant S protein comprises a K to T mutation at a residue corresponding to nucleotide 417 of SEQ ID NO: 37, and a E to K mutation at a residue corresponding to nucleotide 484 of SEQ ID NO: 37, and a N to Y mutation at a residue corresponding to nucleotide 501 of SEQ ID NO: 37.

In one example, the mutant S protein comprises (i) a T to I mutation at a residue corresponding to nucleotide 95 of SEQ ID NO: 37; and/or (ii) a Y to S mutation at a residue corresponding to nucleotide 144 of SEQ ID NO: 37; and/or (iii) a Y to N mutation at a residue corresponding to nucleotide 145 of SEQ ID NO: 37; and/or (iv) a R to K mutation at a residue corresponding to nucleotide 346 of SEQ ID NO: 37; and/or (v) an E to K mutation at a residue corresponding to nucleotide 484 of SEQ ID NO: 37; and/or (vi) a N to Y mutation at a residue corresponding to nucleotide 501 of SEQ ID NO: 37; and/or (vii) a D to G mutation at a residue corresponding to nucleotide 614 of SEQ ID NO: 37; and/or (viii) a P to H mutation at a residue corresponding to nucleotide 681 of SEQ ID NO: 37; and/or (ix) a D to N mutation at a residue corresponding to nucleotide 950 of SEQ ID NO: 37.

In one example, the mutant S protein comprises a T to I mutation at a residue corresponding to nucleotide 95 of SEQ ID NO: 37.

In one example, the mutant S protein comprises a Y to S mutation at a residue corresponding to nucleotide 144 of SEQ ID NO: 37.

In one example, the mutant S protein comprises a Y to N mutation at a residue corresponding to nucleotide 145 of SEQ ID NO: 37.

In one example, the mutant S protein comprises a R to K mutation at a residue corresponding to nucleotide 346 of SEQ ID NO: 37.

In one example, the mutant S protein comprises a D to N mutation at a residue corresponding to nucleotide 950 of SEQ ID NO: 37.

In one example, the mutant S protein comprises (i) a T to I mutation at a residue corresponding to nucleotide 95 of SEQ ID NO: 37; and (ii) a Y to S mutation at a residue corresponding to nucleotide 144 of SEQ ID NO: 37; and (iii) a Y to N mutation at a residue corresponding to nucleotide 145 of SEQ ID NO: 37; and (iv) a R to K mutation at a residue corresponding to nucleotide 346 of SEQ ID NO: 37; and (v) an E to K mutation at a residue corresponding to nucleotide 484 of SEQ ID NO: 37; and (vi) a N to Y mutation at a residue corresponding to nucleotide 501 of SEQ ID NO: 37; and (vii) a D to G mutation at a residue corresponding to nucleotide 614 of SEQ ID NO: 37; (viii) a P to H mutation at a residue corresponding to nucleotide 681 of SEQ ID NO: 37; (ix) a D to N mutation at a residue corresponding to nucleotide 950 of SEQ ID NO: 37.

In one example, the mutant S protein comprises (i) a T to K mutation at a residue corresponding to nucleotide 478 of SEQ ID NO: 37; and/or (ii) a P to R mutation at a residue corresponding to nucleotide 681 of SEQ ID NO: 37; and/or (iii) a L to R mutation at a residue corresponding to nucleotide 452 of SEQ ID NO: 37.

In one example, the mutant S protein comprises a T to K mutation at a residue corresponding to nucleotide 478 of SEQ ID NO: 37.

In one example, the mutant S protein comprises a P to R mutation at a residue corresponding to nucleotide 681 of SEQ ID NO: 37.

In one example, the mutant S protein comprises a L to R mutation at a residue corresponding to nucleotide 452 of SEQ ID NO: 37.

In one example, the mutant S protein comprises (i) a T to K mutation at a residue corresponding to nucleotide 478 of SEQ ID NO: 37; and (ii) a P to R mutation at a residue corresponding to nucleotide 681 of SEQ ID NO: 37; and (iii) a L to R mutation at a residue corresponding to nucleotide 452 of SEQ ID NO: 37.

In one example, the S protein comprises deletion of two residues corresponding to nucleotides 69 and 70 of SEQ ID NO: 37.

In one example, the S protein comprises deletion of one residue corresponding to nucleotide 144 of SEQ ID NO: 37.

In one example, the S protein (i) comprises RRAR to QQAA mutations at residues corresponding to nucleotides 682-685 of SEQ ID NO: 37; and (ii) comprises deletion of two residues corresponding to nucleotides 69 and 70 of SEQ ID NO: 37; and (iii) comprises deletion of one residue corresponding to nucleotide 144 of SEQ ID NO: 37; and (iv) comprises a N to Y mutation at a residue corresponding to nucleotide 501 of SEQ ID NO: 37; and (v) comprises D to G mutation at residue corresponding to nucleotide 614 of SEQ ID NO: 37. For example, the mutant S protein is encoded by a sequence set forth in SEQ ID NO: 58.

In one example, the S protein comprises deletion of three residues corresponding to nucleotides 242 to 244 of SEQ ID NO: 37.

In one example, the S protein (i) comprises RRAR to QQAA mutations at residues corresponding to nucleotides 682-685 of SEQ ID NO: 37; and (ii) comprises deletion of three residues corresponding to nucleotides 242 to 244 of SEQ ID NO: 37; and (iii) comprises a K to N mutation at a residue corresponding to nucleotide 417 of SEQ ID NO: 37; and (iv) comprises a E to K mutation at a residue corresponding to nucleotide 484 of SEQ ID NO: 37; and (v) comprises a N to Y mutation at a residue corresponding to nucleotide 501 of SEQ ID NO: 37; and (vi) comprises D to G mutation at residue corresponding to nucleotide 614 of SEQ ID NO: 37. For example, the mutant S protein is encoded by a sequence set forth in SEQ ID NO: 59.

In one example, the S protein (i) comprises RRAR to QQAA mutations at residues corresponding to nucleotides 682-685 of SEQ ID NO: 37; and (ii) comprises deletion of two residues corresponding to nucleotides 69 and 70 of SEQ ID NO: 37; and (iii) comprises deletion of three residues corresponding to nucleotides 242 to 244 of SEQ ID NO: 37 and (iv) comprises a K to N mutation at a residue corresponding to nucleotide 417 of SEQ ID NO: 37; and (v) comprises a E to K mutation at a residue corresponding to nucleotide 484 of SEQ ID NO: 37; and (vi) comprises a N to Y mutation at a residue corresponding to nucleotide 501 of SEQ ID NO: 37; and (vii) comprises D to G mutation at residue corresponding to nucleotide 614 of SEQ ID NO: 37. For example, the mutant S protein is encoded by a sequence set forth in SEQ ID NO: 60.

In one example, the S protein comprises an A to D mutation at a residue corresponding to nucleotide 570 of SEQ ID NO: 37.

In one example, the S protein comprises a P to H mutation at a residue corresponding to nucleotide 680 of SEQ ID NO: 37.

In one example, the S protein comprises a T to I mutation at a residue corresponding to nucleotide 716 of SEQ ID NO: 37.

In one example, the S protein (i) comprises RRAR to QQAA mutations at residues corresponding to nucleotides 682-685 of SEQ ID NO: 37; and (ii) comprises deletion of two residues corresponding to nucleotides 69 and 70 of SEQ ID NO: 37; and (iii) comprises deletion of one residue corresponding to nucleotide 144 of SEQ ID NO: 37; and (iv) comprises a N to Y mutation at a residue corresponding to nucleotide 501 of SEQ ID NO: 37; and (v) comprises an A to D mutation at a residue corresponding to nucleotide 570 of SEQ ID NO: 37; and (vi) comprises D to G mutation at residue corresponding to nucleotide 614 of SEQ ID NO: 37; and (vii) comprises a P to H mutation at a residue corresponding to nucleotide 680 of SEQ ID NO: 37; and (viii) comprises a T to I mutation at a residue corresponding to nucleotide 716 of SEQ ID NO: 37. For example, the mutant S protein is encoded by a sequence set forth in SEQ ID NO: 61.

In one example, the S protein comprises a L to F mutation at a residue corresponding to nucleotide 18 of SEQ ID NO: 37.

In one example, the S protein comprises a D to A mutation at a residue corresponding to nucleotide 80 of SEQ ID NO: 37.

In one example, the S protein comprises a D to G mutation at a residue corresponding to nucleotide 215 of SEQ ID NO: 37.

In one example, the S protein comprises an A to V mutation at a residue corresponding to nucleotide 701 of SEQ ID NO: 37.

In one example, the S protein (i) comprises RRAR to QQAA mutations at residues corresponding to nucleotides 682-685 of SEQ ID NO: 37; and (ii) comprises a L to F mutation at a residue corresponding to nucleotide 18 of SEQ ID NO: 37; and (iii) comprises a D to A mutation at a residue corresponding to nucleotide 80 of SEQ ID NO: 37; and (iv) comprises a D to G mutation at a residue corresponding to nucleotide 215 of SEQ ID NO: 37; and (v) comprises deletion of three residues corresponding to nucleotides 242 to 244 of SEQ ID NO: 37; and (vi) comprises a K to N mutation at a residue corresponding to nucleotide 417 of SEQ ID NO: 37; and (vii) comprises a E to K mutation at a residue corresponding to nucleotide 484 of SEQ ID NO: 37; and (viii) comprises a N to Y mutation at a residue corresponding to nucleotide 501 of SEQ ID NO: 37; and (ix) comprises D to G mutation at residue corresponding to nucleotide 614 of SEQ ID NO: 37; and (x) comprises an A to V mutation at a residue corresponding to nucleotide 701 of SEQ ID NO: 37. For example, the mutant S protein is encoded by a sequence set forth in SEQ ID NO: 62.

In one example, the mutant S protein: (i) lacks a furin cleavage site at the S1/S2 boundary and comprises RRAR to QQAA mutations at residues corresponding to nucleotides 682-685 of SEQ ID NO: 37; and/or (ii) lacks a furin cleavage site at the S2′ site; and/or (iii) comprises D to G mutation at residue corresponding to nucleotide 614 of SEQ ID NO: 37; and/or (iv) comprises insertion of two proline residues between residues corresponding to nucleotides 986 and 987 of SEQ ID NO: 37; and/or (v) comprises a N to Y mutation at a residue corresponding to nucleotide 501 of SEQ ID NO: 37; and/or (vi) comprises deletion of two residues corresponding to nucleotides 69 and 70 of SEQ ID NO: 37; and/or (vii) comprises deletion of one residue corresponding to nucleotide 144 of SEQ ID NO: 37; and/or (viii) comprises deletion of three residues corresponding to nucleotides 242 to 244 of SEQ ID NO: 37; and/or (ix) comprises a K to N mutation at a residue corresponding to nucleotide 417 of SEQ ID NO: 37; and/or (x) comprises a E to K mutation at a residue corresponding to nucleotide 484 of SEQ ID NO: 37; and/or (xi) comprises an A to D mutation at a residue corresponding to nucleotide 570 of SEQ ID NO: 37; and/or (xii) comprises a P to H mutation at a residue corresponding to nucleotide 680 of SEQ ID NO: 37; and/or (xiii) comprises a T to I mutation at a residue corresponding to nucleotide 716 of SEQ ID NO: 37; and/or (xix) comprises a L to F mutation at a residue corresponding to nucleotide 18 of SEQ ID NO: 37; and/or (xx); and/or comprises a D to A mutation at a residue corresponding to nucleotide 80 of SEQ ID NO: 37; and/or (xxi) comprises a D to G mutation at a residue corresponding to nucleotide 215 of SEQ ID NO: 37; and/or (xxii) comprises an A to V mutation at a residue corresponding to nucleotide 701 of SEQ ID NO: 37.

In one example, the mutant S protein is encoded by a sequence set forth in any one of SEQ ID NO: 9 or SEQ ID NO: 32 to 36.

In one example, the mutant S protein is encoded by a sequence set forth in any one of SEQ ID NO: 9 or SEQ ID NO: 32 to 36 or SEQ ID NO: 58 to 62.

In one example, the mutant S protein is encoded by a sequence set forth in SEQ ID NO: 9.

In one example, the mutant S protein is encoded by a sequence set forth in SEQ ID NO: 32.

In one example, the mutant S protein is encoded by a sequence set forth in SEQ ID NO: 33.

In one example, the mutant S protein is encoded by a sequence set forth in SEQ ID NO: 34.

In one example, the mutant S protein is encoded by a sequence set forth in SEQ ID NO: 35.

In one example, the mutant S protein is encoded by a sequence set forth in SEQ ID NO: 36.

In one example, the mutant S protein is encoded by a sequence set forth in SEQ ID NO: 58.

In one example, the mutant S protein is encoded by a sequence set forth in SEQ ID NO: 59.

In one example, the mutant S protein is encoded by a sequence set forth in SEQ ID NO: 60.

In one example, the mutant S protein is encoded by a sequence set forth in SEQ ID NO: 61.

In one example, the mutant S protein is encoded by a sequence set forth in SEQ ID NO: 62.

In one example, the present disclosure provides a polynucleotide, wherein the polynucleotide comprises, in order from 5′ to 3′:
a) a first nucleotide sequence encoding a S protein; and a second nucleotide sequence encoding a N protein; or
b) a first nucleotide sequence encoding a N protein; and a second nucleotide sequence encoding a S protein.

In one example, the present disclosure provides a polynucleotide, wherein the polynucleotide comprises, in order from 5′ to 3′, a first nucleotide sequence encoding a S protein; and a second nucleotide sequence encoding a N protein.

In one example, the present disclosure provides a polynucleotide, wherein the polynucleotide comprises, in order from 5′ to 3′, a first nucleotide sequence encoding a S protein operably linked to a regulatory element selected from the group consisting of a Kozak consensus sequence, an IRES, a SG promoter and combinations thereof; and a second nucleotide sequence encoding a N protein operably linked to a regulatory element selected from the group consisting of a SG promoter and an IRES.

In one example, the present disclosure provides a polynucleotide, wherein the polynucleotide comprises, in order from 5′ to 3′, a first nucleotide sequence encoding a N protein; and a second nucleotide sequence encoding a S protein.

In one example, the present disclosure provides a polynucleotide, wherein the polynucleotide comprises, in order from 5′ to 3′, a first nucleotide sequence encoding a N protein operably linked to a regulatory element selected from the group consisting of a Kozak consensus sequence, an IRES, a SG promoter and combinations thereof; and a second nucleotide sequence encoding a S protein operably linked to a regulatory element selected from the group consisting of a SG promoter and an IRES.

In one example, the present disclosure provides a RNA, wherein the RNA comprises, in order from 5′ to 3′:
a) a first nucleotide sequence encoding a S protein; and a second nucleotide sequence encoding a N protein; or
b) a first nucleotide sequence encoding a N protein; and a second nucleotide sequence encoding a S protein.

In one example, the present disclosure provides a RNA, wherein the RNA comprises, in order from 5′ to 3′, a first nucleotide sequence encoding a S protein; and a second nucleotide sequence encoding a N protein.

In one example, the present disclosure provides a RNA, wherein the RNA comprises, in order from 5′ to 3′, a first nucleotide sequence encoding a S protein operably linked to a regulatory element selected from the group consisting of a Kozak consensus sequence, an IRES, a SG promoter and combinations thereof; and a second nucleotide sequence encoding a N protein operably linked to a regulatory element selected from the group consisting of a SG promoter and an IRES.

In one example, the present disclosure provides a RNA, wherein the RNA comprises, in order from 5′ to 3′, a first nucleotide sequence encoding a N protein; and a second nucleotide sequence encoding a S protein.

In one example, the present disclosure provides a RNA, wherein the RNA comprises, in order from 5′ to 3′, a first nucleotide sequence encoding a N protein operably linked to a regulatory element selected from the group consisting of a Kozak consensus sequence, an IRES, a SG promoter and combinations thereof; and a second nucleotide sequence encoding a S protein operably linked to a regulatory element selected from the group consisting of a SG promoter and an IRES.

In one example, the present disclosure provides a cRNA, wherein the cRNA comprises, in order from 5′ to 3′:
a) a first nucleotide sequence encoding a S protein; and a second nucleotide sequence encoding a N protein; or
b) a first nucleotide sequence encoding a N protein; and a second nucleotide sequence encoding a S protein.

In one example, the present disclosure provides a cRNA, wherein the cRNA comprises, in order from 5′ to 3′, a first nucleotide sequence encoding a S protein; and a second nucleotide sequence encoding a N protein.

In one example, the present disclosure provides a cRNA, wherein the cRNA comprises, in order from 5′ to 3′, a first nucleotide sequence encoding a S protein operably linked to a regulatory element selected from the group consisting of a Kozak consensus sequence, an IRES, a SG promoter and combinations thereof; and a second nucleotide sequence encoding a N protein operably linked to a regulatory element selected from the group consisting of a SG promoter and an IRES.

In one example, the present disclosure provides a cRNA, wherein the cRNA comprises, in order from 5′ to 3′, a first nucleotide sequence encoding a N protein; and a second nucleotide sequence encoding a S protein.

In one example, the present disclosure provides a cRNA, wherein the cRNA comprises, in order from 5′ to 3′, a first nucleotide sequence encoding a N protein operably linked to a regulatory element selected from the group consisting of a Kozak consensus sequence, an IRES, a SG promoter and combinations thereof; and a second nucleotide sequence encoding a S protein operably linked to a regulatory element selected from the group consisting of a SG promoter and an IRES.

In one example, the present disclosure provides a multicistronic self-replicating RNA, wherein the RNA comprises, in order from 5′ to 3′:
a) a first nucleotide sequence encoding a S protein; and a second nucleotide sequence encoding a N protein; or
b) a first nucleotide sequence encoding a N protein; and a second nucleotide sequence encoding a S protein.

In one example, the present disclosure provides a multicistronic self-replicating RNA, wherein the RNA comprises, in order from 5′ to 3′, a first nucleotide sequence encoding a S protein; and a second nucleotide sequence encoding a N protein.

In one example, the present disclosure provides a multicistronic self-replicating RNA, wherein the RNA comprises, in order from 5′ to 3′, a first nucleotide sequence encoding a S protein operably linked to a SG promoter; and a second nucleotide sequence encoding a N protein operably linked to a regulatory element selected from the group consisting of a SG promoter and an IRES.

In one example, the present disclosure provides a multicistronic self-replicating RNA, wherein the molecule comprises, in order from 5′ to 3′:
a) a first nucleotide sequence encoding a S protein operably linked to a minimal SG promoter encoded by a sequence set forth in SEQ ID NO: 1; and
b) a second nucleotide sequence encoding a N protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′:
a) a first nucleotide sequence encoding a S protein operably linked to a minimal SG promoter encoded by a sequence set forth in SEQ ID NO: 1; and
b) a second nucleotide sequence encoding a N protein operably linked to an extended SG promoter encoded by a sequence set forth in SEQ ID NO: 2.

In one example, the present disclosure provides a multicistronic self-replicating RNA, wherein the RNA comprises, in order from 5′ to 3′:
a) a first nucleotide sequence encoding a S protein operably linked to a minimal SG promoter encoded by a sequence set forth in SEQ ID NO: 1; and
b) a second nucleotide sequence encoding a N protein operably linked to an extended SG promoter encoded by a sequence set forth in SEQ ID NO: 3.

In one example, the present disclosure provides a multicistronic self-replicating RNA, wherein the molecule comprises, in order from 5′ to 3′:
a) a first nucleotide sequence encoding a mutated S protein operably linked to a minimal SG promoter encoded by a sequence set forth in SEQ ID NO: 1; and
b) a second nucleotide sequence encoding a N protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′:
a) a first nucleotide sequence encoding a mutated S protein operably linked to a minimal SG promoter encoded by a sequence set forth in SEQ ID NO: 1; and
b) a second nucleotide sequence encoding a N protein operably linked to an extended SG promoter encoded by a sequence set forth in SEQ ID NO: 2.

In one example, the present disclosure provides a multicistronic self-replicating RNA, wherein the RNA comprises, in order from 5′ to 3′:
a) a first nucleotide sequence encoding a mutated S protein operably linked to a minimal SG promoter encoded by a sequence set forth in SEQ ID NO: 1; and
b) a second nucleotide sequence encoding a N protein operably linked to an extended SG promoter encoded by a sequence set forth in SEQ ID NO: 3.

In one example, the present disclosure provides a multicistronic self-replicating RNA, wherein the RNA comprises, in order from 5′ to 3′:
a) a first nucleotide sequence encoding a S protein operably linked to a minimal SG promoter encoded by a sequence set forth in SEQ ID NO: 1; and
b) a second nucleotide sequence encoding a N protein operably linked to an IRES encoded by a sequence set forth in SEQ ID NO: 4.

In one example, the present disclosure provides a multicistronic self-replicating RNA, wherein the RNA comprises, in order from 5′ to 3′, a first nucleotide sequence encoding a N protein; and a second nucleotide sequence encoding a S protein.

In one example, the present disclosure provides a multicistronic self-replicating RNA, wherein the RNA comprises, in order from 5′ to 3′, a first nucleotide sequence encoding a N protein operably linked to a SG promoter; and a second nucleotide sequence encoding a S protein operably linked to a regulatory element selected from the group consisting of a SG promoter and an IRES.

In one example, the present disclosure provides a multicistronic self-replicating RNA, wherein the molecule comprises, in order from 5′ to 3′:
a) a first nucleotide sequence encoding a N protein operably linked to a minimal SG promoter encoded by a sequence set forth in SEQ ID NO: 1; and
b) a second nucleotide sequence encoding a S protein operably linked to a minimal SG promoter encoded by a sequence set forth in SEQ ID NO: 1.

In one example, the present disclosure provides a multicistronic self-replicating RNA, wherein the RNA comprises, in order from 5′ to 3′:
a) a first nucleotide sequence encoding a N protein operably linked to a minimal SG promoter encoded by a sequence set forth in SEQ ID NO: 1; and
b) a second nucleotide sequence encoding a S protein operably linked to an extended SG promoter encoded by a sequence set forth in SEQ ID NO: 2.

In one example, the present disclosure provides a multicistronic self-replicating RNA, wherein the RNA comprises, in order from 5′ to 3′:
a) a first nucleotide sequence encoding a N protein operably linked to a minimal SG promoter encoded by a sequence set forth in SEQ ID NO: 1; and
b) a second nucleotide sequence encoding a S protein operably linked to an extended SG promoter encoded by a sequence set forth in SEQ ID NO: 3.

In one example, the present disclosure provides a multicistronic self-replicating RNA, wherein the RNA comprises, in order from 5′ to 3′:
a) a first nucleotide sequence encoding a N protein operably linked to a minimal SG promoter encoded by a sequence set forth in SEQ ID NO: 1; and
b) a second nucleotide sequence encoding a S protein operably linked to an IRES encoded by a sequence set forth in SEQ ID NO: 4.

In one example, the present disclosure provides a multicistronic self-replicating RNA encoded by a sequence set forth in any one of SEQ ID NO: 10 to 14 or SEQ ID NO: 19 to 27 or SEQ ID NO: 30 to 31. In one example, the present disclosure provides a multicistronic self-replicating RNA encoded by a sequence set forth in any one of SEQ ID NO: 10 to 14 or SEQ ID NO: 19 to 27 or SEQ ID NO: 30 to 31 or SEQ ID NO: 49 to 53. For example, the present disclosure provides a multicistronic self-replicating RNA encoded by a sequence set forth in any one of SEQ ID NO: 10 to 14 or SEQ ID NO: 19 to 27. In another example, the present disclosure provides a multicistronic self-replicating RNA encoded by a sequence set forth in any one of SEQ ID NO: 30 to 31. In another example, the present disclosure provides a multicistronic self-replicating RNA encoded by a sequence set forth in any one of SEQ ID NO: 49 to 53.

In one example, the present disclosure provides a multicistronic self-replicating RNA encoded by a sequence set forth in SEQ ID NO: 10 (F548).

In one example, the present disclosure provides a multicistronic self-replicating RNA encoded by a sequence set forth in SEQ ID NO: 11 (F549).

In one example, the present disclosure provides a multicistronic self-replicating RNA encoded by a sequence set forth in SEQ ID NO: 12 (F556).

In one example, the present disclosure provides a multicistronic self-replicating RNA encoded by a sequence set forth in SEQ ID NO: 13 (F557).

In one example, the present disclosure provides a multicistronic self-replicating RNA encoded by a sequence set forth in SEQ ID NO: 14 (F602).

In one example, the present disclosure provides a multicistronic self-replicating RNA encoded by a sequence set forth in SEQ ID NO: 19 (F554).

In one example, the present disclosure provides a multicistronic self-replicating RNA encoded by a sequence set forth in SEQ ID NO: 20 (F568).

In one example, the present disclosure provides a multicistronic self-replicating RNA encoded by a sequence set forth in SEQ ID NO: 21 (F569).

In one example, the present disclosure provides a multicistronic self-replicating RNA encoded by a sequence set forth in SEQ ID NO: 22 (F570).

In one example, the present disclosure provides a multicistronic self-replicating RNA encoded by a sequence set forth in SEQ ID NO: 23 (F576).

In one example, the present disclosure provides a multicistronic self-replicating RNA encoded by a sequence set forth in SEQ ID NO: 24 (F584).

In one example, the present disclosure provides a multicistronic self-replicating RNA encoded by a sequence set forth in SEQ ID NO: 25 (F590).

In one example, the present disclosure provides a multicistronic self-replicating RNA encoded by a sequence set forth in SEQ ID NO: 26 (F616).

In one example, the present disclosure provides a multicistronic self-replicating RNA encoded by a sequence set forth in SEQ ID NO: 27 (F620).

In one example, the present disclosure provides a multicistronic self-replicating RNA encoded by a sequence set forth in SEQ ID NO: 30 (Col8).

In one example, the present disclosure provides a multicistronic self-replicating RNA encoded by a sequence set forth in SEQ ID NO: 31 (Col9).

In one example, the present disclosure provides a multicistronic self-replicating RNA encoded by a sequence set forth in SEQ ID NO: 49 (F631).

In one example, the present disclosure provides a multicistronic self-replicating RNA encoded by a sequence set forth in SEQ ID NO: 50 (F632).

In one example, the present disclosure provides a multicistronic self-replicating RNA encoded by a sequence set forth in SEQ ID NO: 51 (F629).

In one example, the present disclosure provides a multicistronic self-replicating RNA encoded by a sequence set forth in SEQ ID NO: 52 (F695).

In one example, the present disclosure provides a multicistronic self-replicating RNA encoded by a sequence set forth in SEQ ID NO: 53 (703).

The present disclosure provides an immunogenic composition comprising the polynucleotide of the present disclosure. The present disclosure further provides an immunogenic composition comprising the RNA of the present disclosure. For example, the present disclosure provides an immunogenic composition comprising the cRNA of the present disclosure. The present disclosure also provides an immunogenic composition comprising the self-replicating RNA of the present disclosure. For example, the composition of the present disclosure, when administered, is capable of inducing an immune response in the subject. For example, administration of the composition induces a humoral and/or a cell-mediated immune response. In one example, the composition induces a humoral immune response in the subject. For example, the humoral immune response is an antibody-mediated immune response. In another example, the composition induces a cell-mediated immune response. For example, the cell-mediated immune response includes activation of antigen-specific cytotoxic T cells.

In one example, the immunogenic composition of the disclosure comprises multiple polynucleotides, wherein each polynucleotide encodes different polypeptide antigen sequences. In another example, the immunogenic composition of the disclosure comprises multiple RNAs, wherein each RNA encodes different polypeptide antigen sequences. In a further example, the immunogenic composition of the disclosure comprises multiple cRNAs, wherein each cRNA encodes different polypeptide antigen sequences. In one example, the immunogenic composition comprises multiple multicistronic self-replicating RNAs, wherein each multicistronic self-replicating RNA encodes different polypeptide antigen sequences. For example, the different polypeptide antigen sequences are from the same virus (e.g., encode antigens from the same influenza A virus strain). In one example, the different polypeptide antigen sequences are from different viruses. For example, the sequences encode different influenza A virus strains.

The present disclosure also provides a pharmaceutical composition comprising an immunogenic composition of the present disclosure and a pharmaceutically acceptable carrier. Pharmaceutically acceptable carriers suitable for use in the present disclosure will be apparent to the skilled person and/or are described herein.

In one example, the pharmaceutical composition further comprises a lipid nanoparticle (LNP), a polymeric microparticle, and an oil-in-water emulsion. For example, the polynucleotide, the RNA, the cRNA or the self-replicating RNA is encapsulated in, bound to or adsorbed on a LNP, a polymeric microparticle, and an oil-in-water emulsion. In one example, the polynucleotide is encapsulated in, bound to or adsorbed on a LNP, a polymeric microparticle, and an oil-in-water emulsion. In another example, the RNA is encapsulated in, bound to or adsorbed on a LNP, a polymeric microparticle, and an oil-in-water emulsion. For example, the cRNA is encapsulated in, bound to or adsorbed on a LNP, a polymeric microparticle, and an oil-in-water emulsion. For example, the self-replicating RNA is encapsulated in, bound to or adsorbed on a LNP, a polymeric microparticle, and an oil-in-water emulsion.

In one example, the pharmaceutical composition further comprises a LNP. For example, the polynucleotide is encapsulated in a LNP. In another example, the RNA is encapsulated in a LNP. For example, the cRNA is encapsulated in a LNP. For example, the self-replicating RNA is encapsulated in a LNP. For example, the polynucleotide is bound to a LNP. In another example, the RNA is bound to a LNP. For example, the cRNA is bound to a LNP. In another example, the self-replicating RNA is bound to a LNP. For example, the polynucleotide is adsorbed on to a LNP. In another example, the RNA is adsorbed on to a LNP. For example, the cRNA is adsorbed on to a LNP. In a further example, the self-replicating RNA is adsorbed on to a LNP.

In one example, the LNP comprises a PEG-lipid, a structural lipid and/or a neutral lipid. For example, the LNP comprises a PEG-lipid, a structural lipid and a neutral lipid. In another example, the LNP comprises a PEG-lipid, a structural lipid or a neutral lipid.

In one example, the LNP further comprises a cationic lipid. In another example, the LNP does not comprise a cationic lipid.

In one example, the pharmaceutical composition further comprises a polymeric microparticle. For example, the polynucleotide is encapsulated in a polymeric microparticle. In another example, the RNA is encapsulated in a polymeric microparticle. For example, the cRNA is encapsulated in a polymeric microparticle. For example, the self-replicating RNA is encapsulated in a polymeric microparticle. For example, the polynucleotide is bound to a polymeric microparticle. In another example, the RNA is bound to a polymeric microparticle. For example, the cRNA is bound to a polymeric microparticle. In another example, the self-replicating RNA is bound to a polymeric microparticle. For example, the polynucleotide is adsorbed on to a polymeric microparticle. In another example, the RNA is adsorbed on to a polymeric microparticle. For example, the cRNA is adsorbed on to a polymeric microparticle. In a further example, the self-replicating RNA is adsorbed on to a polymeric microparticle.

In one example, the pharmaceutical composition further comprises an oil-in-water emulsion. For example, the polynucleotide is encapsulated in an oil-in-water emulsion. In another example, the RNA is encapsulated in an oil-in-water emulsion. For example, the cRNA is encapsulated in an oil-in-water emulsion. For example, the self-replicating RNA is encapsulated in an oil-in-water emulsion. For example, the polynucleotide is bound to an oil-in-water emulsion. In another example, the RNA is bound to an oil-in-water emulsion. For example, the cRNA is bound to an oil-in-water emulsion. In another example, the self-replicating RNA is bound to an oil-in-water emulsion. In a further example, the self-replicating RNA is adsorbed on to an oil-in-water emulsion. In a further example, the self-replicating RNA is resuspended in an oil-in-water emulsion.

The present disclosure also provides the immunogenic composition or the pharmaceutical composition of the disclosure for use as a vaccine.

In one example, the polynucleotide is DNA. In one example, the disclosure provides a DNA encoding a cRNA vaccine of the disclosure. In one example, the disclosure provides a DNA encoding a self-replicating RNA vaccine of the disclosure.

In one example, the DNA is a plasmid.

The present disclosure further provides the immunogenic composition or the pharmaceutical composition of the disclosure for use in the treatment or prevention or delaying progression of a respiratory viral infection. For example, the disclosure provides the immunogenic composition or the pharmaceutical composition of the disclosure for use in the treatment of a respiratory viral infection. In one example, the disclosure provides the immunogenic composition or the pharmaceutical composition of the disclosure for use in the prevention of a respiratory viral infection. In another example, the disclosure provides the immunogenic composition or the pharmaceutical composition of the disclosure for use in delaying the progression of a respiratory viral infection. For example, an immunogenic composition or the pharmaceutical composition of the disclosure is for use in the treatment or prevention or delaying progression of influenza, an influenza virus infection, bronchiolitis, pneumonia, croup, a SARS-CoV-2 infection, COVID-19 and/or ARDS. In one example, the immunogenic composition or the pharmaceutical composition of the disclosure is for use in the treatment or prevention or delaying progression of influenza, an influenza virus infection, a SARS-CoV-2 infection, COVID and/or ARDS.

In one example, the present disclosure provides the immunogenic composition or the pharmaceutical composition of the disclosure for use in the treatment or prevention or delaying progression of influenza. For example, the disclosure provides the immunogenic composition or the pharmaceutical composition of the disclosure for use in the treatment of influenza. In another example, the disclosure provides the immunogenic composition or the pharmaceutical composition of the disclosure for use in the prevention of influenza. In a further example, the disclosure provides the immunogenic composition or the pharmaceutical composition of the disclosure for use in delaying the progression of influenza.

In one example, the present disclosure provides the immunogenic composition or the pharmaceutical composition of the disclosure for use in the treatment or prevention or delaying progression of an influenza virus infection. For example, the disclosure provides the immunogenic composition or the pharmaceutical composition of the disclosure for use in the treatment of an influenza virus infection. In another example, the disclosure provides the immunogenic composition or the pharmaceutical composition of the disclosure for use in the prevention of an influenza virus infection. In a further example, the disclosure provides the immunogenic composition or the pharmaceutical composition of the disclosure for use in delaying the progression of an influenza virus infection.

In one example, the present disclosure provides the immunogenic composition or the pharmaceutical composition of the disclosure for use in the treatment or prevention or delaying progression of COVID-19. For example, the disclosure provides the immunogenic composition or the pharmaceutical composition of the disclosure for use in the treatment of COVID-19. In another example, the disclosure provides the immunogenic composition or the pharmaceutical composition of the disclosure for use in the prevention of COVID-19. In a further example, the disclosure provides the immunogenic composition or the pharmaceutical composition of the disclosure for use in delaying the progression of COVID-19.

In one example, the present disclosure provides the immunogenic composition or the pharmaceutical composition of the disclosure for use in the treatment or prevention or delaying progression of a SARS-CoV-2 infection. For example, the disclosure provides the immunogenic composition or the pharmaceutical composition of the disclosure for use in the treatment of a SARS-CoV-2 infection. In another example, the disclosure provides the immunogenic composition or the pharmaceutical composition of the disclosure for use in the prevention of a SARS-CoV-2 infection. In a further example, the disclosure provides the immunogenic composition or the pharmaceutical composition of the disclosure for use in delaying the progression of a SARS-CoV-2 infection.

In one example, the present disclosure provides the immunogenic composition or the pharmaceutical composition of the disclosure for use in the treatment or prevention or delaying progression of ARDS. For example, the disclosure provides the immunogenic composition or the pharmaceutical composition of the disclosure for use in the treatment of ARDS. In another example, the disclosure provides the immunogenic composition or the pharmaceutical composition of the disclosure for use in the prevention of ARDS. In a further example, the disclosure provides the immunogenic composition or the pharmaceutical composition of the disclosure for use in delaying progression of ARDS.

The present disclosure provides a method of treating or preventing or delaying progression of a disease or condition in a subject, the method comprising administering the immunogenic composition or the pharmaceutical composition of the present disclosure to a subject in need thereof. In one example, the disclosure provides a method of treating a disease or condition in a subject, the method comprising administering the immunogenic composition or the pharmaceutical composition of the present disclosure to a subject in need thereof. In another example, the disclosure provides a method of preventing a disease or condition in a subject, the method comprising administering the immunogenic composition or the pharmaceutical composition of the present disclosure to a subject in need thereof. In a further example, the disclosure provides a method of delaying progression of a disease or condition in a subject, the method comprising administering the immunogenic composition or the pharmaceutical composition of the present disclosure to a subject in need thereof.

In one example, the present disclosure provides use of a polynucleotide of the disclosure in the manufacture of a medicament for treating or preventing or delaying progression of a disease or condition in a subject in need thereof. For example, the disclosure provides use of a polynucleotide of the disclosure in the manufacture of a medicament for treating a disease or condition in a subject in need thereof. In another example, the disclosure provides use of a polynucleotide of the disclosure in the manufacture of a medicament for preventing a disease or condition in a subject in need thereof. In a further example, the disclosure provides use of a polynucleotide of the disclosure in the manufacture of a medicament for delaying progression of a disease or condition in a subject in need thereof.

In one example, the present disclosure provides use of a RNA of the disclosure in the manufacture of a medicament for treating or preventing or delaying progression of a disease or condition in a subject in need thereof. For example, the disclosure provides use of a RNA of the disclosure in the manufacture of a medicament for treating a disease or condition in a subject in need thereof. In another example, the disclosure provides use of a RNA of the disclosure in the manufacture of a medicament for preventing a disease or condition in a subject in need thereof. In a further example, the disclosure provides use of a RNA of the disclosure in the manufacture of a medicament for delaying progression of a disease or condition in a subject in need thereof.

In one example, the present disclosure provides use of a cRNA of the disclosure in the manufacture of a medicament for treating or preventing or delaying progression of a disease or condition in a subject in need thereof. For example, the disclosure provides use of a cRNA of the disclosure in the manufacture of a medicament for treating a disease or condition in a subject in need thereof. In another example, the disclosure provides use of a cRNA of the disclosure in the manufacture of a medicament for preventing a disease or condition in a subject in need thereof. In a further example, the disclosure provides use of a cRNA of the disclosure in the manufacture of a medicament for delaying progression of a disease or condition in a subject in need thereof.

In one example, the present disclosure provides use of a self-replicating RNA of the disclosure in the manufacture of a medicament for treating or preventing or delaying progression of a disease or condition in a subject in need thereof. For example, the disclosure provides use of a self-replicating RNA of the disclosure in the manufacture of a medicament for treating a disease or condition in a subject in need thereof. In another example, the disclosure provides use of a self-replicating RNA of the disclosure in the manufacture of a medicament for preventing a disease or condition in a subject in need thereof. In a further example, the disclosure provides use of a self-replicating RNA of the disclosure in the manufacture of a medicament for delaying progression of a disease or condition in a subject in need thereof.

In one example, the subject suffers from a disease or condition. In one example, the subject has been diagnosed as suffering from a disease or condition. In one example, the subject is receiving treatment for a disease or condition.

In one example, the disease or condition is a respiratory viral infection. For example, the respiratory viral infection is selected from the group consisting of influenza, an influenza virus infection, bronchiolitis, pneumonia, croup, a SARS-CoV-2 infection, COVID-19 and ARDS. In one example, the disease or condition is influenza, an influenza virus infection, a SARS-CoV-2 infection, COVID-19 and/or ARDS. In one example, the disease or condition is influenza. In another example, the disease or condition is an influenza virus infection. In another example, the disease or condition is bronchiolitis. In a further example, the disease or condition is pneumonia. In one example, the disease or condition is croup. In another example, the disease or condition is a SARS-CoV-2 infection. In another example, the disease or condition is COVID-19. In a further example, the disease or condition is ARDS. In one example, the ARDS is associated with influenza, an influenza virus infection, a SARS-CoV-2 infection and/or COVID-19.

In one example of any method described herein, the self-replicating RNA of the present disclosure is administered before or after the development of influenza, an influenza virus infection, a SARS-CoV-2 infection, COVID-19 and/or ARDS in a subject. In one example of any method described herein, the self-replicating RNA of the present disclosure is administered before the development of influenza, an influenza virus infection, a SARS-CoV-2 infection, COVID-19 and/or ARDS in a subject. In one example of any method described herein, the self-replicating RNA of the present disclosure is administered after the development of influenza, an influenza virus infection, a SARS-CoV-2 infection, COVID-19 and/or ARDS in a subject.

In one example of any method described herein, the self-replicating RNA of the present disclosure is administered after the detection of a respiratory viral infection. For example, the self-replicating RNA of the present disclosure is administered after the detection of influenza, an influenza virus infection, a SARS-CoV-2 infection, COVID-19 and/or ARDS in a subject. In one example of any method described herein, the self-replicating RNA of the present disclosure is administered after the detection of influenza, an influenza virus infection, a SARS-CoV-2 infection, COVID-19 and/or ARDS in a subject. In a further example of any method described herein, the self-replicating RNA of the present disclosure is administered after the detection of an influenza virus infection. In one example of any method described herein, the self-replicating RNA of the present disclosure is administered after the detection of an influenza virus infection but prior to the development of influenza. In another example of any method described herein, the self-replicating RNA of the present disclosure is administered after the detection of a SARS-CoV-2 infection. In one example, the self-replicating RNA of the present disclosure is administered after the detection of a SARS-CoV-2 infection but prior to the development of COVID-19. In a further example of any method described herein, the self-replicating RNA of the present disclosure is administered after the detection of COVID-19. In one example of any method described herein, the self-replicating RNA of the present disclosure is administered after the detection of COVID-19 but prior to the development of ARDS. In another example of any method described herein, the self-replicating RNA of the present disclosure is administered after the detection of ARDS.

In one example, the subject is at risk of developing influenza, COVID-19 or ARDS. For example, the subject is at risk of developing influenza. In another example, the subject is at risk of developing COVID-19. In a further example, the subject is at risk of developing ARDS.

In one example, the composition of the present disclosure is administered in an amount sufficient to reduce the severity of or prevent onset of one or more symptoms of influenza, an influenza virus infection, a SARS-CoV-2 infection, COVID-19 and/or ARDS. Symptoms of influenza, an influenza virus infection, a SARS-CoV-2 infection, COVID-19 and/or ARDS will be apparent to the skilled person and/or are described herein.

The present disclosure provides a method of inducing an immune response in a subject, comprising administering the self-replicating RNA, the immunogenic composition or the pharmaceutical composition of the present disclosure to a subject in need thereof.

The present disclosure also provides use of the self-replicating RNA, the immunogenic composition or the pharmaceutical composition of the present disclosure in the manufacture of a medicament for inducing an immune response in a subject in need thereof.

In one example, the self-replicating RNA, the immunogenic composition or the pharmaceutical composition of the present disclosure induces a humoral and/or a cell-mediated immune response. In one example, the composition induces a humoral immune response in the subject. For example, the humoral immune response is an antibody-mediated immune response. For example, production of neutralizing antibodies. In another example, the composition induces a cell-mediated immune response. For example, the cell-mediated immune response includes activation of antigen-specific cytotoxic T cells. For example, the T cells are CD4 T cells and/or CD8 T cells. In one example, the T cells are CD4 T cells. In another example the T cells are CD8 T cells. In a further example, the T cells are CD4 and CD8 T cells.

In one example, administration of the self-replicating RNA, the immunogenic composition or the pharmaceutical composition of the present disclosure induces a CD4 T cell mediated immune response.

In one example, administration of the self-replicating RNA, the immunogenic composition or the pharmaceutical composition of the present disclosure induces a CD8 T cell mediated immune response.

In one example, administration of the self-replicating RNA, the immunogenic composition or the pharmaceutical composition of the present disclosure induces a CD4 and a CD8 T cell mediated immune response.

The present disclosure also provides a polynucleotide that encodes the self-replicating RNA of the present disclosure. For example, the polynucleotide is a recombinant DNA. In one example, the recombinant DNA is a plasmid. In one example, the plasmid comprises a sequence set forth in any one of SEQ ID NO: 10 to 14 or SEQ ID NO: 19 to 27 or SEQ ID NO: 30 to 31.

The present disclosure also provides a kit comprising at least one self-replicating RNA of the disclosure, optionally in a delivery system and/or a pharmaceutically acceptable carrier or diluent, packaged with instructions for use in treating or preventing or delaying progression of a disease or disorder (e.g., influenza, an influenza virus infection, a SARS-CoV-2 infection, COVID-19 and/or ARDS) in a subject.

The present disclosure also provides a kit comprising at least one self-replicating RNA of the disclosure, optionally in a delivery system and/or a pharmaceutically acceptable carrier or diluent, packaged with instructions to administer the RNA to a subject who is suffering from or at risk of suffering from a disease or disorder (e.g., influenza, an influenza virus infection, a SARS-CoV-2 infection, COVID-19 and/or ARDS).

In one example, the self-replicating RNA, the immunogenic composition or the pharmaceutical composition of the disclosure is supplied in a vial. In another example, the self-replicating RNA, the immunogenic composition or the pharmaceutical composition of the disclosure is supplied in a syringe.

### BRIEF DESCRIPTION OF THE DRAWINGS

FIG. 1 (A) is a schematic representation of a self-replicating RNA prepared using HA and NA subtypes derived from A/turkey/Turkey/1/2005. (B) and (C) illustrate the 5′-cap driven antigen expression in the constructs.

FIG. 2 illustrates the pattern of gene expression of the H5 and N1 genes of interest in the unformulated RNA constructs (A) F548 (B) F549 (C) F602 (D) F616 (E) F556 (F) F557 (G) F568 (H) F569 (I) F576 (J) F620 (K) F584 (L) F590 as determined by mean fluorescence intensity (MFI) analysis.

FIG. 3 illustrates the pattern of gene expression of the first and second genes of interest of the RNA formulated in lipid nanoparticles as determined by mean fluorescence intensity analysis. (A) Expression of H5 and N1 antigens in F556 and F548 constructs. (B) Expression of H5 and N1 antigens in F557 and F549 constructs. (C) Expression of H5 antigen in F556, F602 and F616 constructs as compared to control construct F500.3 expressing H5 antigen alone. (D) Expression of N1 antigen in F556, F602 and F616 constructs as compared to control construct F543 expressing N1 antigen alone. (E) Expression of H5, N1 and M1 antigens in F554 construct. (F) Expression of H5, N1 and M1 antigens in F584 construct. (G) Expression of H5, N1, M1 and M2 antigens in F590 construct.

FIG. 4 illustrates the microneutralization titres from mice immunized with the self-replicating RNA in (A) short and (B) long form microneutralization assays FIG. 5 illustrates hemagglutinin titres from mice immunized with the self-replicating RNA.

FIG. 6 illustrates antigen specific CD4 and CD8 T cell responses. (A) H5 and N1 antigen specific CD8 T cell responses in F548, F549, F556 and F557. (B) H5 and N1 antigen specific CD8 T cell responses in F556, F557, F602 and F616. (C) H5 and N1 antigen specific CD4 T cell responses in F548, F549, F556 and F557. (D) H5 and N1 antigen specific CD4 T cell responses in F556, F557, F602 and F616.

FIG. 7 illustrates (A) antibody responses as assessed by microneutralization assay and (B) inhibition of ACE2 binding.

FIG. 8 illustrates antigen specific CD4 and CD8 T cell responses. (A) S specific CD4 T cell responses with Pep Mix 1 (white bars) and Pep Mix 2 (black bars). (B) S specific CD8 T cell responses with Pep Mix 1 (white bars) and Pep Mix 2 (black bars). (C) N specific CD4 T cell responses and (D) N specific CD8 T cell responses.

FIG. 9 is a series of graphical representations showing antigen-specific T cells induced by Col8. The net (antigen-specific) % cytokine-producing CD4 and CD8 T cells induced are shown for (A) Si-specific CD4 T cells, (B) Si-specific CD8 T cells (C) S2-specific CD4 T cells, (D) S2-specific CD8 T cells, and (E) N-specific CD4 T cells.

FIG. 10 is a series of graphical representations showing (A) net % antigen-specific CD4+ responses; (B) net % antigen-specific CD8+ response; and normalized frequency of (C) antigen-specific CD4 responses and (D) antigen-specific CD8 responses.

KEY TO SEQUENCE LISTING

| | |
|---|---|
| SEQ ID NO: 1 | Nucleotide sequence of alphavirus native subgenomic |
| | promoter |
| SEQ ID NO: 2 | Nucleotide sequence of extended subgenomic promoter (v2) |
| SEQ ID NO: 3 | Nucleotide sequence of extended subgenomic promoter (v3) |
| SEQ ID NO: 4 | Nucleotide sequence of wild-type EMCV IRES |
| SEQ ID NO: 5 | Nucleotide sequence of influenza A virus H5 hemagglutinin |
| | subtype (A/turkey/Turkey/1/2005) |
| SEQ ID NO: 6 | Nucleotide sequence of influenza A virus N1 neuraminidase |
| | subtype (A/turkey/Turkey/1/2005) |
| SEQ ID NO: 7 | Nucleotide sequence of SARS-CoV-2 nucleocapsid (N) protein |
| | full length wt |
| SEQ ID NO: 8 | Nucleotide sequence of SARS-CoV-2 spike (S) protein full |
| | length wt (cleavable) |
| SEQ ID NO: 9 | Nucleotide sequence of SARS-CoV-2 mutated spike (S) |
| | protein uncleavable (S1/S2 RRAR to QQAA mutation) |
| SEQ ID NO: 10 | Nucleotide sequence of construct F548 |
| SEQ ID NO: 11 | Nucleotide sequence of construct F549 |
| SEQ ID NO: 12 | Nucleotide sequence of construct F556 |
| SEQ ID NO: 13 | Nucleotide sequence of construct F557 |
| SEQ ID NO: 14 | Nucleotide sequence of construct F602 |
| SEQ ID NO: 15 | Nucleotide sequence of extended subgenomic promoter (v4) |
| SEQ ID NO: 16 | Nucleotide sequence of influenza A virus M1 matrix protein |
| | (PR8-X) |
| SEQ ID NO: 17 | Nucleotide sequence of influenza A virus M2 matrix protein |
| SEQ ID NO: 18 | Nucleotide sequence of influenza A virus NS1 non-structural |
| | protein (A/California/09) |
| SEQ ID NO: 19 | Nucleotide sequence of construct F554 |
| SEQ ID NO: 20 | Nucleotide sequence of construct F568 |
| SEQ ID NO: 21 | Nucleotide sequence of construct F569 |
| SEQ ID NO: 22 | Nucleotide sequence of construct F570 |
| SEQ ID NO: 23 | Nucleotide sequence of construct F576 |
| SEQ ID NO: 24 | Nucleotide sequence of construct F584 |
| SEQ ID NO: 25 | Nucleotide sequence of construct F590 |
| SEQ ID NO: 26 | Nucleotide sequence of construct F616 |
| SEQ ID NO: 27 | Nucleotide sequence of construct F620 |
| SEQ ID NO: 28 | Nucleotide sequence of influenza virus nucleoprotein |
| | (A/California/09) |
| SEQ ID NO: 29 | Nucleotide sequence of influenza A virus M1 matrix protein |
| | (A/California/09) |
| SEQ ID NO: 30 | Nucleotide sequence of construct Co18 |
| SEQ ID NO: 31 | Nucleotide sequence of construct Co19 |
| SEQ ID NO: 32 | Nucleotide sequence of SARS-CoV-2 spike (S) protein |
| | uncleavable (S1/S2 RRAR to QQAA mutation and 986P/987P |
| | mutation) |
| SEQ ID NO: 33 | Nucleotide sequence of SARS-CoV-2 spike (S) protein |
| | uncleavable (S1/S2 RRAR to QQAA mutation and D614G |
| | mutation) |
| SEQ ID NO: 34 | Nucleotide sequence of SARS-CoV-2 spike (S) protein |
| | uncleavable (S1/S2 RRAR to QQAA mutation and S2′ |
| | mutation) |
| SEQ ID NO: 35 | Nucleotide sequence of SARS-CoV-2 spike (S) protein |
| | uncleavable (S1/S2 RRAR to QQAA mutation and D614G |
| | mutation and S2′ mutation) |
| SEQ ID NO: 36 | Nucleotide sequence of SARS-CoV-2 spike (S) protein |
| | cleavable (D614G mutation) |
| SEQ ID NO: 37 | Amino acid sequence of SARS-CoV-2 S protein full length wt |
| SEQ ID NO: 38 | Nucleotide sequence of a Kozak consensus sequence |
| SEQ ID NO: 39 | Nucleotide sequence of a Kozak consensus sequence |
| SEQ ID NO: 40 | Nucleotide sequence of an interrupting linker |
| SEQ ID NO: 41 | Nucleotide sequence of a GC-rich element |
| SEQ ID NO: 42 | Nucleotide sequence of a GC-rich element |
| SEQ ID NO: 43 | Nucleotide sequence of a GC-rich element |
| SEQ ID NO: 44 | Nucleotide sequence of a histone stem loop |
| SEQ ID NO: 45 | Nucleotide sequence of 5′UTR of VEEV |
| SEQ ID NO: 46 | Nucleotide sequence of 3′UTR of SINV |
| SEQ ID NO: 47 | Nucleotide sequence of extended subgenomic promoter |
| SEQ ID NO: 48 | Poly-A sequence |
| SEQ ID NO: 49 | Nucleotide sequence of construct 631 |
| SEQ ID NO: 50 | Nucleotide sequence of construct 632 |
| SEQ ID NO: 51 | Nucleotide sequence of construct 629 |
| SEQ ID NO: 52 | Nucleotide sequence of construct 695 |
| SEQ ID NO: 53 | Nucleotide sequence of construct 703 |
| SEQ ID NO: 54 | Nucleotide sequence of influenza A virus H3 protein |
| | (A/Delaware/39/2019) |
| SEQ ID NO: 55 | Nucleotide sequence of influenza A virus N2 protein |
| | (A/Delaware/39/2019) |
| SEQ ID NO: 56 | Nucleotide sequence of influenza B virus Hyam |
| | (B/Singapore/INFTT 16 0610/16 (By)) |
| SEQ ID NO: 57 | Nucleotide sequence of influenza B virus Nyam |
| | (B/Singapore/INFTT 16 0610/16 (By)) |
| SEQ ID NO: 58 | Nucleotide sequence of SARS-CoV-2 spike (S) protein |
| | (RRAR→QQAA; Δ69-70; ΔY144; N501Y; D614G) |
| SEQ ID NO: 59 | Nucleotide sequence of SARS-CoV-2 spike (S) protein |
| | (RRAR→QQAA; Δ242-244; K417N; E484K; N501Y; D614G) |
| SEQ ID NO: 60 | Nucleotide sequence of SARS-CoV-2 spike (S) protein |
| | (RRAR→QQAA; Δ69-70; A242-244; K417N; E484K; |
| | N501Y; D614G) |
| SEQ ID NO: 61 | Nucleotide sequence of SARS-CoV-2 spike (S) protein |
| | (RRAR→QQAA; Δ69-70; ΔY144; N501Y; A570D; D614G; |
| | P680H; T716I) |
| SEQ ID NO: 62 | Nucleotide sequence of SARS-CoV-2 spike (S) protein |
| | (RRAR→QQAA; L18F; D80A; D215G; Δ242-244; K417N; |
| | E484K; N501Y; D614G; A701V) |

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

### Selected Definitions

As used herein, the term “multicistronic” (also known as “polycistronic”) in reference to the polynucleotide, RNA, cRNA and/or self-replicating RNA, refers to a RNA that encodes two or more polypeptides. The term encompasses “bicistronic” (or “dicistronic”; i.e., encoding two polypeptides) and “tricistronic” (i.e., encoding three polypeptides) molecules. By “bicistronic” is meant a single nucleic acid that is capable of encoding two distinct polypeptides from different regions of the nucleic acid.

As used herein, the term “conventional mRNA” or “cRNA” or “non-amplifying RNA” refers to a construct that allows expression of heterologous RNA and proteins but the RNA that cannot amplify in host cells.

As used herein, the term “self-replicating RNA” refers to a construct based on an RNA virus that has been engineered to allow expression of heterologous mRNA and proteins. Self-replicating RNA (e.g., in the form of naked RNA) can amplify in host cells leading to expression of the desired gene product in the host cell.

The term “naked” as used herein refers to nucleic acids that are substantially free of other macromolecules, such as lipids, polymers and proteins. A “naked” nucleic acid, such as a self-replicating RNA, is not formulated with other macromolecules to improve cellular uptake. Accordingly, a naked nucleic acid is not encapsulated in, absorbed on, or bound to a lipid nanoparticle (LNP), a liposome, a polymeric microparticle or an oil-in-water emulsion.

As used herein, the term “nucleotide sequence” or “nucleic acid sequence” will be understood to mean a series of contiguous nucleotides (or bases) covalently linked to a phosphodiester backbone. By convention, sequences are presented from the 5′ end to the 3′ end, unless otherwise specified. To facilitate a clear description of the nucleic acids, particular sequence components are referred to as e.g., a “first nucleotide sequence” and a “second nucleotide sequence”. It is to be understood that the first and second sequences can appear in any desired order or orientation, unless otherwise specified, and that no particular order or orientation is intended by the words “first”, “second” etc.

As used herein, the term “antigen” refers to a molecule or structure containing one or more epitopes that induce, elicit, augment or boost a cellular and/or humoral immune response. Antigens can include, for example, proteins and peptides from a pathogen such as a virus, bacteria, fungus, protozoan, plant or from a tumour.

As used herein, the term “operably linked to” means positioning a subgenomic promoter or regulatory element (e.g., an IRES) relative to a nucleic acid such that expression of the nucleic acid is controlled or regulated by the element. For example, a subgenomic promoter can be operably linked to numerous nucleic acids, e.g., through another regulatory element, such as an internal ribosome entry site (IRES).

As used herein, the term “subgenomic promoter” (also known as ‘junction region’ promoter) refers to a promoter that directs the expression of a heterologous nucleotide sequence, regulating protein expression.

As used herein, the term “internal ribosome entry site” or “IRES” refers to a sequence of nucleotides within a mRNA to which a ribosome or a component thereof, e.g., a 40S subunit of a ribosome, is capable of binding. An IRES need not necessarily comprise nucleic acid that induces translation of a mRNA (e.g., a start codon; AUG).

The term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

The term “recombinant” shall be understood to mean the product of artificial genetic recombination.

As used herein the term “substantially the same” in reference to the level of expression is meant that the first and second antigens (at least) have a level of expression within about 10% or less of each other.

As used herein, the terms “disease”, “disorder” or “condition” refers to a disruption of or interference with normal function, and is not to be limited to any specific condition, and will include diseases or disorders.

As used herein, a subject “at risk” of developing a disease or condition may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a RNA or composition described herein to thereby reduce or eliminate at least one symptom of a specified disease or condition.

As used herein, the term “preventing”, “prevent” or “prevention” includes providing prophylaxis with respect to occurrence or recurrence of a specified disease or condition in an individual. An individual may be predisposed to or at risk of developing the disease but has not yet been diagnosed with the disease.

As used herein, the phrase “delaying progression of” includes reducing or slowing down the progression of the disease or condition in an individual and/or at least one symptom of a disease or condition.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of RNA. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the RNA of the present disclosure to elicit a desired response in the individual. A therapeutically effective amount is also one in which any toxic or detrimental effects of the RNA are outweighed by the therapeutically beneficial effects.

As used herein, the term “prophylactically effective amount” shall be taken to mean a sufficient quantity of the RNA of the disclosure to prevent or inhibit or delay the onset of one or more detectable symptoms of a disease or disorder as described herein.

As used herein, the term “subject” shall be taken to mean any animal including humans, for example a mammal. Exemplary subjects include but are not limited to humans and non-human primates. For example, the subject is a human.

As used herein, the term “lipid nanoparticle” or “LNP” shall be understood to refer to lipid-based particles having at least one dimension on the order of nanometers (e.g., 1-1,000 nm) and which comprises a compound of any formulae described herein. In embodiments, LNPs are formulated in a composition for delivery of a polynucleotide to a desired target such as a cell, tissue, organ, tumor, and the like. For example, the lipid nanoparticle or LNP any lipid composition, including, may be selected from, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers.

### Polynucleotides

As used herein, the term “polynucleotide” refers a molecular chain of nucleotides chemically bonded by a series of ester linkages between the phosphoryl group of one nucleotide and the hydroxyl group of the sugar in an adjacent nucleotide. In one example, the polynucleotide is a DNA. In one example, the polynucleotide is a RNA, e.g., mRNA. For example, the mRNA is a conventional mRNA (cRNA) or a self-replicating RNA.

As used herein, the term “fragment” refers to a portion of a nucleotide sequence or polypeptide of a reference nucleotide sequence or polypeptide disclosed herein which maintains a defined activity of the full length nucleotide sequence or polypeptide.

As used herein, the term “variant” refers to a nucleotide sequence with one or more substitutions, insertions, deletions and/or other modifications compared to the unmodified sequence. It will be apparent to the skilled person that any variant described herein will have the same or similar expression of the encoded protein. For example, the variant is a functional variant. Exemplary modifications to the nucleotide sequence and/or polypeptide will be apparent to the skilled person and/or described herein.

In one example, a modification is a chemical modification of one or more nucleotide(s) of the nucleotide sequence. For example, at least one naturally occurring nucleotide of the polynucleotide is replaced with a chemically modified nucleotide (e.g. pseudouridine (ψ) and 1-methylpseudouridine (m1ψ)).

In one example, the modification comprises increasing the G/C content of the nucleotide sequence.

In one example, the modification comprises codon optimization of the nucleotide sequence.

In one example, the substitution is a conservative substitution. A skilled person will appreciate that a conservative substitution with reference to a polypeptide involves replacement of an amino acid in the polypeptide with a different amino acid with similar biochemical properties (e.g. charge, hydrophobicity and size). In one example, the substitution is a non-conservative substitution.

As used herein, the term “encode”, “encodes” or “encoding” refers to a region of a polynucleotide capable of undergoing translation into a polypeptide.

The polynucleotide of the present disclosure includes DNA and RNA (e.g. mRNA).

### Deoxyribonucleic Acid (DNA)

In one example, the polynucleotide is a DNA (e.g. DNA vector).

It will be apparent to the skilled person that a DNA of the present disclosure further comprises an endonuclease restriction site at the 3′ end of the 3′UTR. The skilled person will appreciate that endonuclease restriction site allows for the insertion of one or more nucleotide sequence(s) (e.g. encoding an antigen of interest, a fragment and/or a variant thereof) without disrupting the remainder of the DNA.

As used herein, the term “restriction endonuclease site” refers to a sequence of DNA that binds to a restriction endonuclease. Typically, the restriction endonuclease site is short sequence (e.g. of approximately 4-8 base pairs) recognised and cleaved by the restriction endonuclease.

As used herein, the term “restriction enzymes” or “restriction endonucleases” refers to a class of enzyme that occur naturally in bacteria and in some viruses. Restriction endonuclease bind specifically to and cleave double-stranded DNA at specific sites within or adjacent to a restriction endonuclease site. Exemplary restriction endonuclease include, for example, BciVI (Bful), Bcul (Spel), EcoRI, Aatll, AgeI (BshTI), Apal, BamHI, BglII, Blpl (Bpu11021), BsrGI (Bsp1407), Clal (Bsu15I), EcoRI, EcoRV (Eco32I), Eam1104I (Earl), Hindlll, Kpnl, Mlul, Ncol, Ndel, Nhel, Notl, Nsil, Mph1103I), Pstl, Pvul, Pvull, SacI, SalI, ScaI, SpeI, Xbal, Xhol, Sacll (Cfr42I) and Xbal.

In one example, the present disclosure provides a transcribable polynucleotide comprising the first nucleotide sequence encoding a first antigen of interest; and the second nucleotide sequence encoding a second antigen of interest operably linked to a regulatory element selected from the group consisting of a SG promoter and an IRES. For example, the polynucleotide is the DNA plasmid comprising the first and second nucleotide sequences and optionally one or more nucleotide sequence(s) encoding one or more antigens of interest.

In one example, the DNA comprises a nucleotide sequence comprising a restriction endonuclease site located 3′ of the 3′UTR. The presence of the restriction endonuclease site located 3′ of the 3′UTR allows for production of a linearised DNA. Linearisation of DNA ensures defined termination of in vitro transcribed DNA to produce mRNA.

### Ribonucleic Acid (RNA)

In one example, the polynucleotide is a mRNA comprising, in the order of 5′ to 3′ the first nucleotide sequence encoding a first antigen of interest; and the second nucleotide sequence encoding a second antigen of interest operably linked to a regulatory element selected from the group consisting of a SG promoter and an IRES.

The mRNA of the present disclosure encompasses a non-replicating mRNA (also referred to as conventional mRNA (cRNA) or non-amplifying) and a self-replicating RNA (also known as self-amplifying RNA or sa-mRNA).

### Conventional (Non-Replicating) RNA

The present disclosure provides a multicistronic cRNA.

The skilled person will understand that the cRNA of the present disclosure comprise in order from 5′ to 3′: a 5′cap structure, a 5′-UTR, a fragment and/or a variant thereof, a first nucleotide sequence encoding a first antigen of interest, a second nucleotide sequence encoding a second antigen of interest, a 3′-UTR and a 3′tailing sequence (e.g. a polyadenylation signal or one or more poly-A tails). The cRNA of the present disclosure may further comprise an translation internal ribosome entry site (e.g. Kozak consensus sequence or IRES) operably linked to the first antigen of interest.

### Self-Replicating RNA

The present disclosure provides a multicistronic self-replicating RNA (also known as a replicon).

The skilled person will understand that the self-replicating RNA of the present disclosure is based on the genomic RNA of RNA viruses. The RNA should be positive (+)-stranded so that it can be directly translated after delivery to a cell without the need for intervening replication steps (e.g., reverse transcription). Translation of the RNA results in the production of non-structural proteins (NSPs) which combine to form a replicase complex (i.e., an RNA-dependent RNA polymerase). The complex then amplifies the original RNA, producing both antisense and sense transcripts, resulting in production of multiple daughter RNAs which may subsequently be translated and transcribed, enhancing overall protein expression.

In one example, the self-replicating RNA of the present disclosure comprises the non-structural proteins of the RNA virus, the 5′ and 3′ untranslated regions (UTRs) and the native subgenomic promoter.

In one example, the self-replicating RNA comprises one or more non-structural proteins of the RNA virus. For example, the RNA comprises at least one or more genes selected from the group consisting of a viral replicase (or viral polymerase), a viral protease, a viral helicase and other non-structural viral proteins. For example, the self-replicating RNA comprises a viral replicase (or viral polymerase).

In another example, the self-replicating RNA comprises a 5′- and a 3′-end UTR of the RNA virus. It will be apparent to the skilled person that the terms 5′ and a 3′UTR also encompasses the terms 5′ and 3′ conserved sequence elements (CSE). In one example, the self-replicating RNA comprises a 5′- and a 3′-end CSE.

The self-replicating RNA of the present disclosure cannot induce production of infectious viral particles. For example, the self-replicating RNA of the present disclosure does not comprise viral genes encoding structural proteins necessary for production of viral particles.

In one example, the self-replicating RNA is derived from or based on an alphavirus. Suitable alphaviruses will be apparent to the skilled person and/or described herein.

In another example, the self-replicating RNA is derived from or based on a virus other than an alphavirus, for example, a positive-stranded RNA virus. Suitable positive-stranded RNA viruses suitable for use in the present disclosure will be apparent to the skilled person and include, for example, a picornavirus, a flavivirus, a rubivirus, a pestivirus, a hepacivirus, a calicivirus, or a coronavirus.

### Alphavirus

In one example, the self-replicating RNA of the present disclosure is derived from (or based on) an alphavirus.

Alphaviruses are the sole genus in the Togaviridae family and are an enveloped virus with a positive-sense, single-stranded RNA genome. The skilled person will understand that the alphavirus genome comprises two open reading frames (ORFs), non-structural and structural. The first ORF encodes four non-structural proteins (NSP1, NSP2, NSP3 and NSP4) necessary for transcription and replication of viral RNA. The second encodes three structural proteins: the core nucleocapsid protein C, and the envelope proteins P62 and E1, which associate as a heterodimer. The viral membrane-anchored surface glycoproteins are responsible for receptor recognition and entry into target cells through membrane fusion.

In one example, the self-replicating RNA of the present disclosure comprises a viral replicase (or viral polymerase). For example, the viral replicase is an alphavirus replicase, such as an alphavirus protein NSP4.

In one example, the self-replicating RNA of the present disclosure does not encode one or more alphavirus structural proteins (e.g., capsid and/or envelope glycoproteins). For example, the self-replicating RNA is unable to produce RNA-containing alphavirus virions (i.e., infectious viral particles).

In one example, the self-replicating RNA comprises a native alphavirus SG promoter. For example, the native alphavirus SG promoter is a minimal SG promoter (i.e., the minimal sequence required for initiation of transcription) and comprises a sequence set forth in SEQ ID NO: 1.

The skilled person will be aware of alphaviruses suitable for use in the present disclosure. Exemplary alphaviruses include, but are not limited to, Venezuelan equine encephalitis virus (VEE; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus.

### Regulatory Elements

The present disclosure provides a polynucleotide comprising a first nucleotide sequence encoding a first antigen of interest and a second nucleotide sequence encoding a sequence antigen of interest operably linked to a regulatory element selected from the group consisting of a SG promoter and an IRES.

The present disclosure provides a RNA (e.g., a cRNA or self-replicating RNA) comprising a first nucleotide sequence encoding a first antigen of interest and a second nucleotide sequence encoding a sequence antigen of interest operably linked to a regulatory element selected from the group consisting of a SG promoter and an IRES.

In one example, the first antigen of interest is operably linked to to a regulatory element selected from the group consisting of a Kozak consensus sequence, an IRES, a SG promoter and combinations thereof.

The present disclosure provides a self-replicating RNA comprising a first nucleotide sequence encoding a first antigen operably linked to a subgenomic (SG) promoter; and a second nucleotide sequences encoding a second antigen operably linked to a regulatory element a promoter selected from the group consisting of a SG promoter and an internal ribosome entry site (IRES).

### Kozak Consensus Sequence

As used herein, the term “Kozak consensus sequence” refers to a nucleotide sequence identified in eukaryotic genes that facilitates the translation of the gene by containing a start codon (also referred to as a translation initiation codon) which is recognised by a ribosome.

Exemplary Kozak consensus sequence are known in the art and/or described herein. In one example, the Kozak consensus sequence is set forth in SEQ ID NO: 38. In another example, the Kozak consensus sequence is set forth in SEQ ID NO: 39. In one example, the Kozak consensus sequence is ACCATGG. In another example, the Kozak consensus sequence is ACCATG.

### Subgenomic Promoters

SG promoters (also known as ‘junction region’ promoters) suitable for use in the present disclosure will be apparent to the skilled person and/or are described herein.

In one example, the SG promoter is derived from or based on an alphavirus SG promoter. For example, the SG promoter is a native alphavirus SG promoter. In one example, the native SG promoter is a minimal SG promoter. For example, the minimal SG promoter is the minimal sequence required for initiation of transcription. In one example, the native SG promoter is an extended SG promoter. For example, the extended SG promoter is a minimal SG promoter extended at the 5′ end with nucleotides occurring in a sequence encoding a non-structural protein (e.g., NSP4) of the RNA virus (e.g., an alphavirus). In one example, the extended SG promoter is a minimal SG promoter extended at the 5′ end with nucleotides occurring in a sequence encoding an alphavirus NSP4.

In one example, the polynucleotide of the disclosure comprises a SG promoter from any alphavirus. For example, the RNA of the disclosure (e.g., cRNA or self-replicating RNA) comprises a SG promoter from any alphavirus.

In one example, the self-replicating RNA comprises a SG promoter from any alphavirus.

The polynucleotide of the present disclosure comprises two or more nucleotide sequences encoding two or more antigens of interest. In one example, the two or more nucleotide sequences are each operably linked to SG promoters. When two or more SG promoters are present in the RNA of the present disclosure, the promoters can be the same or different. For example, the two or more SG promoters are derived from the same alphavirus. In another example, the two or more SG promoters are derived from different alphaviruses.

When two or more SG promoters are present in the self-replicating RNA of the present disclosure, the promoters can be the same or different. For example, the two or more SG promoters are derived from the same alphavirus. In another example, the two or more SG promoters are derived from different alphaviruses.

### Internal Ribosomal Entry Site (IRES)

IRES suitable for use in the present disclosure will be apparent to the skilled person and/or are described herein.

In one example, the IRES is derived from encephalomyocarditis virus (EMCV). For example, the IRES is a wild-type IRES from EMCV.

In one example, the IRES is derived from a fibroblast growth factor 1A (FGF1A) IRES.

In addition, synthetic IRES elements have been described, which can be designed, according to methods know in the art to mimic the function of naturally occurring IRES elements (see Chappell, S A et al. Proc. Natl Acad. Sci. USA (2000) 97(4): 1536-41).

### 5′Untranslated Region (5′-UTR)

The present disclosure provides a polynucleotide comprising a first nucleotide sequence comprising a 5′-untranslated region (5′-UTR).

As used herein, the term “5′-untranslated region” or “5′-UTR” refers to a non-coding region of an mRNA located at the 5′end of the translation initiation sequence (AUG).

Exemplary 5′-UTRs include, for example, 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 45.

In one example, the 5′UTR comprises at least one microRNA binding site, an AU rich element (ARE), a GC-rich element, a stem loop, and combinations thereof.

microRNA Binding Site

As used herein, the term “microRNA binding site” refers to a sequence within a polynucleotide (e.g. within a DNA or RNA transcript) that has sufficient complementarity to all or one region of a miRNA to interact, associate or bind to the microRNA (miRNA).

As used herein, the term “microRNA” or “miRNA” refers to 19-25 nucleotide long non-coding RNAs that bind to the 5′-UTR of polynucleotides and down-regulate gene expression (e.g. by inhibiting translation). The presence of microRNA binding site(s) in the 5′UTR of the present disclosure can function to inhibit translation of the 5′-UTR.

Suitable miRNA binding sites for use in the present disclosure will be apparent to the skilled person and/or described herein.

In one example, the miRNA binding site comprises a binding site for tissue specific microRNA or those regulating biological processes. For example, miRNA of the liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-id, miR-149), kidney (miR-192, miR-194, miR-204), and lung epithelial cells (let-7, miR-133, miR-126). For example, microRNA that regulate biological processes such as angiogenesis (miR-132). Further exemplifying miRNA and miRNA binding sites are disclosed in US patent application U.S. Ser. No. 14/043,927.

### AU Rich Element (ARE)

As used herein, the term “AU rich element (ARE)” or “AU rich elements (AREs)” refers to a region of a nucleotide sequence comprising stretches of Adeonisine (A) and Uridine (U). Exemplary AREs include, for example, ARE from cytoplasmic myc (c-myc), myoblast determination protein 1 (myoD), c-Jun, Myogenin, granulocyte-macrophage colony-stimulating factor (GM-CSF) and tumour necrosis factor alpha (TNF-α), or a combination thereof.

In one example, the ARE comprises a human antigen R or “HuR” (also known as Elavl1) specific binding site. HuR is known to bind AREs increasing the stability of the mRNA.

### GC-Rich Element

As used herein, the term “GC-rich element” refers to a nucleotide sequence with a high amount of Guanine (G) and/or Cytosine (C) compared to Adenine (A) and Thymine (T)/Uracil (U). The presence of GC-rich elements in a polynucleotide (e.g. mRNA) can stabilise the mRNA.

In one example, the GC-rich element comprises a sequence of 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30 nucleotides in length.

In one example, the GC-rich element comprises between 30% and 40%, or 40% and 50%, or 50% and 60%, or 60% and 70% cytosine. For example, the GC-rich element comprises between 30% and 40% cytosine. For example, the GC-rich element comprises between 40% and 50% cytosine. For example, the GC-rich element comprises between 50% and 60% cytosine. For example, the GC-rich element comprises between 60% and 70% cytosine.

In one example, the GC-rich element comprises 30%, or 40%, or 50%, or 60%, or 70% cytosine. For example, the GC-rich element comprise 30% cytosine. For example, the GC-rich element comprises 40% cytosine. For example, the GC-rich element comprises 50% cytosine. For example, the GC-rich element comprises 60% cytosine. For example, the GC-rich element comprises 60% cytosine. For example, the GC-rich element comprises 70% cytosine.

In one example, the GC-rich element is at least 50% cytosine.

In one example, the GC-rich element is at least 60% cytosine.

In one example, the GC-rich element is at least 70% cytosine.

In one example, the GC-rich element comprises a nucleotide sequence CCCCGGCGCC. In another example, the GC-rich element comprises a nucleotide sequence CCCCGGC. In a further example, the GC-rich element comprises a nucleotide sequence GCGCCCCGCGGCGCCCCGCG.

In one example, the GC-rich element comprises a nucleotide sequence set forth in SEQ ID NO: 41 to 43. In one example, the GC-rich element comprises a nucleotide sequence set forth in SEQ ID NO: 41. In another example, the GC-rich element comprises a nucleotide sequence set forth in SEQ ID NO: 42. In a further example, the GC-rich element comprises a nucleotide sequence set forth in SEQ ID NO: 43.

### Stem Loop

As used herein, the term “stem loop” refers to a nucleotide sequence comprising an intramolecular base pairing of two neighboured entirely or partially reverse complementary sequences to form a stem-loop. A stem-loop can occur in single-stranded DNA or, more commonly, in RNA. The stem loop can also be referred to as a hairpin or hairpin loop which usually consists of a stem and a terminal loop within a consecutive sequence, wherein the stem is formed by two neighboured entirely or partially reverse complementary sequences separated by a short sequence which builds the loop into a stem-loop structure.

The stability of the paired stem loop is determined by the length, the number of mismatched or bulges it contains, and the nucleotide composition of the paired region.

In one example, a loop of the stem loop is between 3 and 10 nucleotides in length. For example, the loop of the stem loop is between 3 and 8, or 3 and 7, or 3 and 6, or 4 and 5 nucleotides in length.

In one example, the loop of the stem loop is 4 nucleotides in length.

In one example, the stem loop is a histone stem loop. For example, the histone stem loop comprises or consist of a nucleotide sequence set for in SEQ ID NO: 44.

### 3′Untranslated Region (3′-UTR)

The present disclosure provides a polynucleotide comprising a 3′-untranslated region (3′-UTR).

As used herein, the term “3′-UTR” refers to a region of an mRNA located at the 3′end of the translation termination codon (i.e. stop codon).

Exemplary 3′-UTRs include, for example, a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 46.

In one example, the 3′-UTR comprises or consists of a nucleotide sequence derived from a 3′-UTR of an albumin gene. In one example, the 3′-UTR comprises or consists of a nucleotide sequence derived from a 3′-UTR of a vertebrate α-globin gene. For example, the 3′-UTR comprises or consists of a nucleotide sequence derived from a 3′-UTR of a mammalian α-globin gene. For example, the 3′-UTR comprises or consists of a nucleotide sequence derived from a 3′-UTR of a human α-globin gene.

In one example, the 3′-UTR of the present disclosure further comprises at least one microRNA binding site, an AU rich element (ARE), a GC-rich element, a triple helix, a stem loop, one or more stop codons or a combination thereof.

### Stop Codon

As used herein, the term “stop codon” refers to a trinucleotide sequence within a mRNA that signals the stop of protein synthesis by a ribosome.

In one example, the polynucleotide of the present disclosure comprises at least one stop codon at the 5′end of a 3′-UTR. For example, the stop codon is selected from UAG, UAA, and UGA.

In one example, the polynucleotide comprises two consecutive stop codons comprising a sequence UGAUGA.

In one example, the polynucleotide comprises two consecutive stop codons comprising a sequence UAAUAG.

### 3′ Tailing Sequence

The polynucleotide of the present disclosure comprises one or more 3′ tailing sequences located at the 3′end of the 3′UTR.

As described herein, the term “3′ tailing sequence” or “3′ tailing sequences” refers to a nucleotide sequence (e.g. polyadenylation signal) which induces the addition of non-encoded nucleotides to the 3′end of a mRNA or a nucleotide sequence (e.g. poly-A sequence) located at the 3′ end of a mRNA. A skilled person will appreciate that the 3′tailing sequence and/or products of the 3′tailing sequence in a mRNA functions to stabilise the mRNA and/or prevent the mRNA from degradation.

As used herein, the term “interrupting linker” in reference to a poly-A or poly-C sequence of the present disclosure refers to a single nucleotide or nucleotide sequence which are linked to, and interrupt, a stretch of consecutive adenosine or cytosine nucleotides in the poly-A or poly-C sequence. For example, the interrupting linker in a poly-A sequence is a single nucleotide or a nucleotide sequence consisting or comprising a nucleotide other than an adenosine nucleotide. For example, the interrupting linker in a poly-C sequence is a single nucleotide or a nucleotide sequence consisting or comprising a nucleotide other than an cytosine nucleotide.

In one example, the one or more 3′ tailing sequences are selected from the group consisting of a poly-A sequence, polyadenylation signal, a G-quadruplex, a poly-C sequence, a stem loop and combinations thereof.

### Poly-A Sequence

As used herein, the term “polyA sequence” refers to a nucleotide sequence of Adenine (A) located at the 3′end of a mRNA. In the context of the present disclosure, the polyA sequence may be located within the mRNA or DNA (e.g. a DNA plasmid serving as a template for generating the mRNA by transcription of the vector).

Suitable poly-A sequence for use in the present disclosure will be apparent to the skilled person and/or are described herein. In one example, the poly-A sequence comprises consecutive (i.e. one after the other) adenosine nucleotides of any length (e.g. to 10 to 300). In one example, the poly-A sequence comprises consecutive adenosine nucleotides separated by one or more interrupting linkers. In one example, the poly-A sequence comprises consecutive adenosine nucleotides without an interrupting linker.

### Polyadenylation Signal

As used herein, the term “polyadenylation signal” refers to a nucleotide sequence which induces polyadenylation. Polyadenylation is typically understood to be the addition of a polyA sequence to a RNA (e.g. to a premature mRNA to generate a mature mRNA). The polyadenylation signal may be located within a nucleotide sequence at the 3′-end of the polynucleotide (e.g. mRNA) to be polyadenylated.

Suitable polyadenylation signal for use in the present disclosure will be apparent to the skilled person and/or described herein.

In one example, the polyadenylation signal comprises a hexamer consisting of Adenine and Uracil/Thymidine nucleotides. In one example, the hexamer sequence comprises or consists of AAUAAA.

In one example, the 3′tailing sequence comprises a polyadenylation signal but does not comprise a polyA sequence.

### G-Quadruplex

As used herein, the term “G-quadruplex” or “G4” refers to a nucleotide sequence rich in guanine residues which forms a four stranded secondary structure. For example, the G-quadruplex is a cyclic hydrogen bonded array of four guanine nucleotides formed by G-rich sequences in both DNA and RNA.

In one example, the 3′ tailing sequence comprises a polyA sequence and a G-quadruplex. For example, the 3′ tailing sequence comprises a polyA sequence linked to a G-quadruplex to produce a polyA-G quartet.

### Poly-C Sequence

As used herein, the term “poly-C sequence” refers to a nucleotide sequence of Cytosine (C) located at the 3′end of a mRNA. In the context of the present disclosure, the polyC sequence may be located within the mRNA or DNA (e.g. a DNA plasmid serving as a template for generating the mRNA by transcription of the vector).

Suitable poly-C sequence for use in the present disclosure will be apparent to the skilled person and/or are described herein.

In one example, the one or more 3′ tailing sequences comprises one or more poly-C sequences each comprising between 10 and 300 consecutive cytosine nucleotides. For example, the one or more poly-C sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive cytosine nucleotides. For example, the one or more poly-C sequence each comprises 10, or 20, or 30, or 40, or 50, or 60, or 70, or 80, or 90, or 100, or 125, or 150, or 175, or 200, or 225, or 250, or 275, or 300 consecutive cytosine nucleotides.

In one example, the one or more poly-C sequences is separated by an interrupting linker. For example, the fourth nucleotide sequence comprising the one or more 3′tailing sequences comprises, in order of 5′ to 3′: consecutive cytosine nucleotides, an interrupting linker, and further consecutive cytosine nucleotides.

In one example, the interrupting linker is from 10 to 50, or 50 to 100, or 100 to 150 nucleotides in length. For example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length.

### 5′Cap Structure

In one example, the present disclosure provides a mRNA comprising a 5′terminal cap structure.

As used herein, the term “5′cap structure” refers to a structure at the 5′ terminal end of a mRNA involved in nuclear export and binds a mRNA Cap Binding Protein (CBP). The 5′cap structure is known to stabilise mRNA through association of CBP with poly(A) binding protein to form a mature mRNA. Accordingly, the presence of a 5′cap structure in the mRNA of the present disclosure can further increase the stability of the mRNA compared to a mRNA without the 5′cap.

Exemplary 5′cap structure includes, for example, anti-reverse cap analogue (ARCA), N7,2′-0-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2.

Typically, an endogenous mRNA is 5′capped with a guanosine through a (5)′-ppp-(5)′-triphosphate linkage attached to the 5′terminal nucleotide of the mRNA. The guanosine cap can then be methylated to a 7-methylguanosine (m7G) generating a 7mG(5′)ppp(5′)N,pN2p (Cap0 structure), where N represents the first and second 5′terminal nucleotide of the mRNA. The cap0 structure can be further 2′-O-methylated to produce 7mG(5′)ppp(5′)NlmpNp (Cap1), and/or 7mG(5′)-ppp(5′)NlmpN2mp (Cap2).

In one example, the polynucleotide of the present disclosure comprises an endogenous cap.

As used herein, the term “endogenous cap” refers to a 5′cap synthesised in a cell. For example, endogenous cap is a natural 5′cap or a wild-type 5′cap. For example, the endogenous cap is a Cap0, Cap1, or Cap2 structure.

In one example, the polynucleotide of the present disclosure comprises an analog of an endogenous cap (also referred to as cap analog).

As used herein, the term “analogue thereof” in the context of an endogenous cap or “cap analog” refers to a synthetic 5′cap. The cap analog can be used to produce 5′capped mRNA in in vitro transcription reactions. Cap analogs may be chemically (i.e. non-enzymatically) or enzymatically synthesized and/or linked to a nucleotide (e.g. 5′terminal nucleotide of an mRNA). Exemplary cap analogs are commercially available and include, for example, 3”-O-Me-m7G(5′)ppp(5′)G, G(5′)ppp(5′)A, G(5′)ppp(5′)G, m7G(5′)ppp(5′)A, m7G(5′)ppp(5′)G (New England BioLabs). In one example, the cap analog is N7,3′-O-dimethyl-guanosine-5′-triphosphate-5′-guanosine (i.e. anti-reverse cap analogue (ARCA)).

In one example, the 5′cap structure is a non-hydrolyzable cap structure. The non-hydrolyzable cap structure can prevent decapping of the mRNA and increase the half-life of the mRNA.

In one example, the non-hydrolyzable cap structure comprises a modified nucleotide selected from a group consisting or a α-thio-guanosine nucleotide, α-methyl-phosphonate, seleno-phosphate, and a combination thereof. In one example, the modified nucleotide is linked to the 5′end of the mRNA through an α-phosphorothiate linkage. Methods of linking the modified nucleotide to the 5′end of the mRNA will be apparent to the skilled person. For example, using a Vaccinia Capping Enzyme (New England Biolabs).

### Modifications

In one example, the polynucleotide of the present disclosure comprises one or more modification(s). Typically, modifications are introduced into a polynucleotide (e.g. mRNA) to increase the translation efficiency and/or stability of the polynucleotide. Suitable modifications to the polynucleotide will be apparent to the skilled person and/or described herein.

In one example, the first nucleotide sequence comprising the 5′-UTR and/or the fragment thereof is modified. Modification of the first nucleotide sequences comprising the 5′-UTR and/or the fragment thereof results in a variant of the 5′-UTR and/or the fragment thereof.

In one example, one or more nucleotide sequence(s) of the polynucleotide are codon optimized. Method of codon optimization will be apparent to the skilled person and/or described herein. For example, tools for codon optimization of polynucleotide include, for example, GeneArt GeneOptimizer (Thermofisher®) or GenSmart® (GeneScript®).

In one example, the polynucleotide is modified to increase the amount of Guanine (G) and/or Cytosine (C) in the polynucleotide. The amount of G/C in the polynucleotide (i.e. G/C content) can influence the stability of the polynucleotide. Accordingly, polynucleotide comprising an increased amount of G/C nucleotides can be functionally more stable than polynucleotides containing a large amount of Adenine (A) and Thymine (T) or Uracil (U) nucleotides. The G/C content is increased by substituting A or T nucleotides with G or C nucleotides.

In one example, the G/C content is increased in the first and/or second nucleotide sequence encoding the first and/or second antigen of interest. In one example, the G/C content is increased in the first and/or second nucleotide sequence encoding the first and/or second antigen of interest and/or the one or more additional nucleotide sequences encoding the one or more antigens of interest. The modification(s) in the first and/or second and/or one or more nucleotide sequences takes advantage of the ability of substituting codons that contain less favourable combinations of nucleotides (in terms of mRNA stability) with alternative codons encoding the same amino acid, or encoding amino acid(s) of similar chemistry (e.g. conserved amino acid substitution). For example, the G/C content is increased by substituting codons containing A or T nucleotides with codons containing G or C nucleotides that encode for the same amino acid. For example, the G/C content is increased by substituting codons containing A or T nucleotides with codons containing G or C nucleotides that encode for an amino acid of similar chemistry.

In one example, the G/C content is increased in one or more nucleotide sequences of the polynucleotide which do not encode the antigen of interest. For example, the G/C content is increased in the 5′-UTR, the fragment and/or the variant thereof. For example, the G/C content is increased in the 3′-UTR, the fragment and/or the variant thereof.

In one example, the polynucleotide comprises at least one chemically modified nucleotide.

As used herein, the term “chemical modification” or “chemical modified” in the context of a nucleotide refers to a naturally occurring nucleotides (i.e. A, T, C, G, U) which are modified by replacement, insertion or removal of individual or several atoms or atomic groups compared to the naturally occurring nucleotides. In one example, at least one naturally occurring nucleotide of the polynucleotide is replaced with a chemically modified nucleotide. In one example, at least 10%, or 20%, or 30%, or 40%, or 50%, or 60%, or 70%, or 80%, or 90%, or 100% of naturally occurring nucleotides of the polynucleotide is replaced with a chemically modified nucleotides. Suitable chemical modified nucleotides for use in the present disclosure will be apparent to the skilled person and/or described herein. Exemplary chemically modified nucleotides include, for example, N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), and 1-methylpseudouridine (m1ψ).

### Antigens

The polynucleotide of the present disclosure comprises a first and second nucleotide sequence that encode a first and second antigen of interest (e.g., a pathogenic antigen). For example, the antigen of interest is an antigen polypeptide, a fragment and/or the variant thereof which can induce an immune response in the subject.

The cRNA of the present disclosure comprises a first and second nucleotide sequence that encode a first and second antigen of interest (e.g., a pathogenic antigen). For example, the antigen of interest is an antigen polypeptide, a fragment and/or the variant thereof which can induce an immune response in the subject.

The self-replicating RNA of the present disclosure comprises a heterologous sequence (e.g., a first and second nucleotide sequence) that encode an antigen (e.g., a pathogenic antigen). For example, the antigen can induce an immune response in the subject.

An antigenic polypeptide, a fragment and/or the variant thereof suitable for use in the polynucleotide described herein will be apparent to the skilled person and, for example, include proteins and peptides derived from any pathogen. For example, the antigen is a virus, bacteria, a fungus, or a protozoan.

Antigens suitable for use in the self-replicating RNA described herein will be apparent to the skilled person and, for example, include proteins and peptides derived from any pathogen. For example, the antigen is a virus, bacteria, a fungus or a protozoan.

### Viral Antigens

In one example, the antigen of the present disclosure is a viral antigen.

Viral antigens that can be encoded by the polynucleotide, the cRNA or the self-replicating RNA will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxovirus types 1-4 (PIV), Mumps, Sendai viruses, Simian virus 5)), Bovine parainfluenza virus, Nipahvirus, Henipavirus and Newcastle disease virus), Poxviridae (e.g., Variola vera, including but not limited to, Variola major and Variola minor, Metapneumoviruses, such as human metapneumovirus (hMPV) and avian metapneumoviruses (aMPV)), Morbilliviruses (e.g., Measles), Picornaviruses (e.g., Enteroviruses, Rhinoviruses, Heparnavirus, Parechovirus, Cardioviruses and Aphthoviruses), Enteroviruseses (e.g., Poliovirus types 1, 2 or 3, Coxsackie A virus types 1 to 22 and 24, Coxsackie B virus types 1 to 6, Echovirus (ECHO) virus types 1 to 9, 11 to 27 and 29 to 34 and Enterovirus 68 to 71), Bunyaviruses (e.g., California encephalitis virus), Phlebovirus (e.g., Rift Valley Fever virus), Nairovirus (e.g., Crimean-Congo hemorrhagic fever virus), Heparnaviruses (e.g., Hepatitis A virus (HAV)), Togaviruses (e.g., Rubivirus, an Alphavirus, or an Arterivirus), Flaviviruses (e.g., Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus), Pestiviruses (e.g., Bovine viral diarrhea (BVDV), Classical swine fever (CSFV) or Border disease (BDV)), Hepadnaviruses (e.g., Hepatitis B virus, Hepatitis C virus), Rhabdoviruses (e.g., Lyssavirus (Rabies virus) and Vesiculovirus (VSV)), Caliciviridae (e.g., Norwalk virus, and Norwalk-like Viruses (e.g., Hawaii Virus and Snow Mountain Virus); Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV), and Porcine transmissible gastroenteritis virus (TGEV)), Retroviruses (e.g., Oncovirus, a Lentivirus or a Spumavirus), Reoviruses (e.g., Orthoreo virus, a Rotavirus, an Orbivirus, or a Coltivirus), Parvoviruses (e.g., Parvovirus B 19), Delta hepatitis virus (HDV), Hepatitis E virus (HEV), Human Herpesviruses (e.g., Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8)), Papovaviruses (e.g., Papillomaviruses and Polyomaviruses), Adenoviruess and Arenaviruses.

In one example, the first and/or second antigen of the present disclosure is a viral antigen from a respiratory virus. Respiratory viral antigens that can be encoded by the self-replicating RNA will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxoviruses (PIV), and Metapneumovirus such as human metapneumovirus (hMPV) and avian metapneumoviruses (aMPV)), Picornaviruses (e.g., Rhinoviruses) and Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV)).

In one example, the first and/or second antigen of the present disclosure is a viral antigen from an influenza virus.

In another example, the first and/or second antigen of the present disclosure is a viral antigen from a coronavirus.

### Bacterial Antigens

In one example, the antigen of the present disclosure is a bacterial antigen.

Bacterial antigens that can be encoded by the polynucleotide, the cRNA or the self-replicating RNA will be apparent to the skilled person and include, for example, proteins and peptides from a Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae), Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enterocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas sp, Klebsiella sp.

### Fungal Antigens

In one example, the antigen of the present disclosure is a fungal antigen.

Fungal antigens that can be encoded by the polynucleotide, the cRNA or the self-replicating RNA will be apparent to the skilled person and include, for example, proteins and peptides from Dermatophytes (including Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme), Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi.

### Protazoan Antigens

In one example, the antigen of the present disclosure is a protazoan antigen.

Protazoan antigens that can be encoded by the polynucleotide, the cRNA or the self-replicating RNA will be apparent to the skilled person and include, for example, proteins and peptides from Entamoeba histolytica, Giardia lambli, Cryptosporidium parvum, Cyclospora cayatanensis and Toxoplasma.

### Methods of Production

Suitable methods for the production of a polynucleotide, a cRNA and/or a self-replicating RNA of the present disclosure will be apparent to the skilled person and/or described herein.

In one example, the polynucleotide is DNA. For example, the polynucleotide is a plasmid DNA.

In one example, the cRNA is produced using a plasmid DNA. In one example, the self-replicating RNA is produced using a plasmid DNA. The skilled person will understand that plasmid DNA is relatively stable. Briefly, competent bacterial cells (e.g., Escherichia coli) cells are transformed with a DNA plasmid encoding a self-replicating RNA of the present disclosure. Individual bacterial colonies are isolated and the resultant plasmid DNA amplified in E. coli cultures.

In one example, the plasmid DNA is isolated following fermentation. For example, the plasmid DNA is isolated using a commercially available kit (e.g., Maxiprep DNA kit), or other routine methods known to the skilled person. Following isolation, plasmid DNA is linearized by restriction digest (i.e., using a restricting enzyme). Restriction enzymes are removed using methods known in the art, including for example phenol/chloroform extraction and ethanol precipitation.

In one example, mRNA is made by in vitro transcription from a linearized DNA template using an RNA polymerase (e.g., T7 RNA polymerase). Following in vitro transcription, the DNA template is removed by DNase digestion. The skilled person will understand that synthetic mRNA capping is performed to correct mRNA processing and contribute to stabilization of the mRNA. In one example, the mRNA is enzymatically 5′-capped. For example, the 5′ cap is a cap0 structure or a cap1 structure. In one example, the 5′ cap is a cap0 structure, for example, the 5′-cap (i.e., cap0) consists of an inverted 7-methylguanosine connected to the rest of the mRNA via a 5′-5′ triphosphate bridge. In one example, the 5′ cap is a cap1 structure, for example, the 5′-cap (i.e., cap1) consists of the cap0 with an additional methylation of the 2′O position of the initiating nucleotide.

In one example, the mRNA is purified. Various methods for purifying mRNA will be apparent to the skilled person. For example, the mRNA is purified using lithium chloride (LiCl) precipitation. In another example, the mRNA is purified using tangential flow filtration (TFF). Following purification, the mRNA is resuspended in e.g., nuclease-free water.

### Compositions

The present disclosure provides an immunogenic composition comprising a polynucleotide of the present disclosure.

The present disclosure also provides an immunogenic composition comprising a cRNA of the present disclosure.

The present disclosure further provides an immunogenic composition comprising a self-replicating RNA of the present disclosure.

The present disclosure also provides a pharmaceutical composition comprising an immunogenic composition of the present disclosure and a pharmaceutically acceptable carrier.

It will be apparent to the skilled person and/or described herein, that the polynucleotide, cRNA and/or self-replicating RNA of the present disclosure may be present as naked RNA or in combination with lipids, polymers or other delivery system that facilitates entry into the cells.

### Delivery Systems

In one example, the pharmaceutical composition of the present disclosure further comprises a LNP, a polymeric microparticle and an oil-in-water emulsion. For example, the polynucleotide, the cRNA and/or the self-replicating RNA is encapsulated in, bound to or adsorbed on a LNP, a polymeric microparticle, or an oil-in-water emulsion.

### Lipid Nanoparticles

In one example, the pharmaceutical composition of the present disclosure further comprises a LNP.

It will be apparent that the term “lipid nanoparticle” or “LNP” refers to any lipid composition, including, but not limited to, liposomes or vesicles, where an aqueous volume is encapsulated by amphipathic lipid bilayers (e.g., single; unilamellar or multiple; multilamellar) micelle-like lipid nanoparticles having a non-aqueous core and solid lipid nanoparticles, wherein solid lipid nanoparticles lack lipid bilayers.

Lipid nanoparticles suitable for use in the present disclosure will be apparent to the skilled person and/or are described herein. The lipids can have an anionic, cationic or zwitterionic hydrophilic head group.

In one example, the lipid nanoparticle comprises a PEG-lipid, a sterol structural lipid and/or a neutral lipid. In one example, the lipid nanoparticle further comprises a cationic lipid. In one example, the lipid nanoparticle does not comprise a cationic lipid.

In one example, the LNP comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one example, the LNP comprises a structural lipid. For example, the structural lipid is selected from the group consisting of cholesterol fecosterol, sitosterol, campesterol, stigmasterol, brassicasterol, ergosterol, tomatidine, tomatine, ursolic acid and alpha-tocopherol and combinations thereof.

In one example, the LNP comprises a neutral lipid. Exemplary phospholipids (anionic or zwitterionic) for use in the present disclosure include, for example, phosphatidylethanolamines, phosphatidylcholines, phosphatidylserines, and phosphatidylglycerols. For example, the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof.

In one example, the LNP comprises a cationic lipid. Exemplary cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750). In one example, the phospholipid is 2,5-bis((9z,12z)-octadeca-9,12,dien-1-yloxyl)benzyl-4-(dimethylamino)butnoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated.

### Polymeric Microparticles

In one example, the pharmaceutical composition of the present disclosure further comprises a polymeric microparticle.

The skilled person will be aware that various polymers can form microparticles to encapsulate or adsorb the polynucleotide, the cRNA and/or the self-replicating RNA of the present disclosure. It will be apparent that use of a substantially non-toxic polymer means that particles are safe, and the use of a biodegradable polymer means that the particles can be metabolised after delivery to avoid long-term persistence. Useful polymers are also sterilisable, to assist in the preparation of pharmaceutical grade formulations.

Exemplary non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

### Oil-In-Water Cationic Emulsions

In one example, the pharmaceutical composition of the present disclosure further comprises an oil-in-water cationic emulsion.

Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will recognise that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: 1, 2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA), 1,2-Dimyristoyl-3-Trimethyl-AmmoniumPropane (DMTAP), dipalmitoyl[C16:0]trimethyl ammonium propane (DPTAP) and distearoyltrimethylammonium propane (DSTAP).

In some examples, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

### Pharmaceutically Acceptable Carrier

Suitably, in compositions or methods for administration of the cRNA and/or the self-replicating RNA of the disclosure to a subject, the cRNA and/or the self-replicating RNA is combined with a pharmaceutically acceptable carrier as is understood in the art. Accordingly, one example of the present disclosure provides a composition (e.g., a pharmaceutical composition) comprising the self-replicating RNA of the disclosure (and any delivery system) combined with a pharmaceutically acceptable carrier. Another example of the present disclosure provides a composition (e.g., a pharmaceutical composition) comprising the cRNA of the disclosure (and any delivery system) combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

The cRNA and/or the self-replicating RNA of the present disclosure is useful for parenteral, topical, oral, or local administration, intramuscular administration, aerosol administration, or transdermal administration, for prophylactic or for therapeutic treatment. In one example, the self-replicating RNA is administered parenterally, such as intramuscularly, subcutaneously or intravenously. For example, the self-replicating RNA is administered intramuscularly. In another example, the cRNA is administered parenterally, such as intramuscularly, subcutaneously or intravenously. For example, the cRNA is administered intramuscularly.

Formulation of a cRNA and/or a self-replicating RNA to be administered will vary according to the route of administration and formulation (e.g., solution, emulsion, capsule) selected. An appropriate pharmaceutical composition comprising a cRNA and/or a self-replicating RNA to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The cRNA and/or self-replicating RNA can be stored in the liquid stage or can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the cRNA and/or self-replicating RNA of the disclosure are those large enough to produce the desired effect. For example, the composition comprises an effective amount of the self-replicating RNA. In one example, the composition comprises a therapeutically effective amount of the self-replicating RNA. In another example, the composition comprises a prophylactically effective amount of the self-replicating RNA. In one example, the composition comprises an effective amount of the cRNA. In one example, the composition comprises a therapeutically effective amount of the cRNA. In another example, the composition comprises a prophylactically effective amount of the cRNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the cRNA and/or the self-replicating RNA is administered at an initial (or loading) dose which is higher than subsequent (maintenance doses). For example, the cRNA and/or the self-replicating RNA is administered at an initial dose of between about 10 mg/kg to about 30 mg/kg. The cRNA and/or the self-replicating RNA is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which the cRNA and/or the self-replicating RNA is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the cRNA and/or the self-replicating RNA of the present disclosure. A subject may be retreated with the cRNA and/or the self-replicating RNA, by being given more than one exposure or set of doses, such as at least about two exposures of the binding protein, for example, from about 2 to 60 exposures, and more particularly about 2 to 40 exposures, most particularly, about 2 to 20 exposures.

In one example, any retreatment may be given when signs or symptoms of disease return.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of the cRNA and/or the self-replicating RNA according to the methods of the present disclosure can be continuous or intermittent, depending, for example, on the recipient's physiological condition, whether the purpose of the administration is therapeutic or prophylactic, and other factors known to skilled practitioners. The administration of the cRNA and/or the self-replicating RNA may be essentially continuous over a preselected period of time or may be in a series of spaced doses, e.g., either during or after development of a condition.

### Screening Assays

Suitable methods for selecting a cRNA and/or a self-replicating RNA of the present disclosure are available to those skilled in the art. Assays may be conducted to assess the efficiency and efficacy of the RNA including, for example, serology and immune responses.

### Antigen Expression

In one example, the self-replicating RNA is assessed for expression of the (at least) first and second genes of interest. In another example, the cRNA is assessed for expression of the (at least) first and second genes of interest.

For example, antigen expression is detected using antibodies against the genes of interest. In one example, the number of cells positive for antigen expression is measured by e.g., fluorescence-activated cell sorting (FACS). In another example the mean fluorescence intensity (MFI) is determined using e.g., FACS. In a further example, the specific potency value or the probability of successful transfection per unit mass of RNA is calculated.

### Microneutralization Assay

In one example, the self-replicating RNA (naked and/or formulated) is assessed for antibody responses. In one example, the cRNA (naked and/or formulated) is assessed for antibody responses. For example, the cRNA and/or the self-replicating RNA is assessed using a microneutralisation assay. Methods of performing a microneutralization assay will be apparent to the skilled person. In one example, the microneutralization assay is a short form assay. For one example, a virus fluorescent focus-based microneutralization assay is performed. In another example, the microneutralization assay is a long form assay.

### Hemagglutination Inhibition (HAI) Assay

In one example, the self-replicating RNA (naked and/or formulated) is assessed for antibody responses. In one example, the cRNA (naked and/or formulated) is assessed for antibody responses. For example, the cRNA and/or self-replicating RNA is assessed using a hemagglutination inhibition (HAI) assay. Methods of performing a HAI assay will be apparent to the skilled person and/or described, for example, in WHO (2011) Manual for the laboratory diagnosis and virological surveillance of influenza: WHO Press, World Health Organization.

### Antigen Specific T Cell Responses

In one example, the self-replicating RNA is assessed for its ability to induce antigen specific T cell responses. In one example, the cRNA is assessed for its ability to induce antigen specific T cell responses. Methods of assessing induction of antigen specific T cell responses will be apparent to the skilled person and/or are described herein.

For example, antigen-specific T cell detection is performed on splenic cultures. Briefly, splenocyte cultures are established in T cell medium and cell cultures are either stimulated with antigenic peptides or unstimulated. In one example, antigen-specific T cell responses are determined using flow cytometry.

### Neutralising Assays

The self-replicating RNA of the disclosure may be screened in vitro for their ability to bind to a SARS-CoV-2 S protein RBD and neutralises binding of the S protein RBD to ACE2. Suitable assays will be apparent to the skilled person and include, for example, a Vero microneutralisation assay, a sVNT assay, or a psuedovirus neutralisation assay (using e.g., HEK-293T cells or HeLa-ACE2 cells).

In one example, the neutralization assay is a Vero microneutralization assay. Briefly, SARS-Cov-2 wild-type virus is passaged in Vero cells (i.e., the Vero lineage isolated from kidney epithelial cells extracted from an African green monkey). Serial two-fold dilutions of a test protein are incubated with 100 TCID₅₀ (i.e., median tissue culture infectious dose) of SARS-CoV-2 for 1 hour and residual virus infectivity is assessed in Vero cells; viral cytopathic effect is read, for example, on day 5. The neutralising antibody titre is calculated using the Reed/Muench method as previously described (Houser et al., 2016; Subbarao et al 2004).

In one example, the neutralization assay is a surrogate neutralization test (sVNT). Briefly, the wells of a plate are coated with hACE2 protein in carbonate-bicarbonate coating buffer (e.g., pH 9.6). HRP-conjugated SARS-CoV-2 and HRP-conjugated SARS-CoV-RBD pre-incubated with test proteins is added to the hACE2 at different concentrations and incubated, for example, for 1 h at room temperature. Unbound HRP conjugated antigens are removed by washing. Colorimetric signal is developed on the enzymatic reaction of HRP with chromogenic substrate, e.g., 3,3′,5,5′-tetramethylbenzidine (TMB). In one example, the absorbance reading at 450 nm and 570 nm is acquired.

In one example, the neutralisation is a psuedovirus neutralisation assay. Briefly, HIV reporter virus pseudotyped with SARS-2-Spike protein is produced by co-transfection of SARS-2-COV-2 spike plasmids together with a viral backbone plasmid (e.g., pDR-NL Aenv FLUC) into e.g., HEK-293T cells. Pseudovirus is harvested post transfection and clarified by filtration. Virus stock titres, reported as Relative Luciferase Units infectious dose (RLU), are calculated by limiting dilution infections in Hela-hACE2 cells measuring luciferase activity as a read-out for viral infection.

### Methods of Treatment or Prevention

The present disclosure provides methods of using the immunogenic composition or the pharmaceutical composition of the present disclosure as a vaccine.

The present disclosure also provides methods of treating or preventing a disease or condition in a subject comprising administering the immunogenic composition or the pharmaceutical composition of the present disclosure. For example, the disease or condition is a respiratory virus infection, such as influenza or COVID-19. In one example, the disease or condition is ARDS.

### Influenza

Influenza, also known as “the flu”, is an infectious disease caused by an influenza virus. Symptoms can be mild to severe and the most common symptoms include high fever, runny nose, sore throat, muscle and joint pain, headache, coughing, and feeling tired. Symptoms typically begin two days after exposure to the virus and most last less than a week. Complications of influenza may include viral pneumonia, secondary bacterial pneumonia, sinus infections, and worsening of previous health problems such as asthma or heart failure. Viral pneumonia may also lead to acute respiratory distress syndrome (ARDS).

It will be apparent to the skilled person that there are currently four influenza viruses—A, B, C and D. Influenza A virus is the most common flu virus infecting humans, animals, and birds, whilst influenza B virus infection mostly occurs in humans. Infection of influenza C virus does not cause any severe symptom in human or mammals and influenza D, to date, has only infected pigs and cattle.

Thus, in some examples of the present disclosure, the subject has an influenza virus infection. In one example, the subject has influenza. In particular, the influenza is associated with ARDS. In one example, the methods of the present disclosure can be used to treat or prevent ARDS in a subject suffering from an influenza virus infection. In one example, the methods of the present disclosure can be used to treat or prevent ARDS in a subject suffering from influenza.

### Coronavirus Disease 2019 (COVID-19)

The present disclosure provides, for example, methods of treating or preventing COVID-19.

The present disclosure also provides, for example, methods of treating or preventing SARS-CoV-2 infection. In some examples of the present disclosure the subject has a SARS-CoV-2 infection but does not have clinically diagnosed COVID-19.

COVID-19 is an infectious disease caused by SARS-CoV-2. It was first identified in December 2019 in Wuhan, Hubei, China, and has resulted in an ongoing pandemic. Common symptoms include fever, cough, fatigue, shortness of breath, and loss of smell and taste. While the majority of cases result in mild symptoms, some progress to ARDS. The time from exposure to onset of symptoms is typically around five days, but may range from two to fourteen days. There are currently no vaccines nor specific antiviral treatments for COVID-19 and management involves the treatment of symptoms, supportive care, isolation, and experimental measures.

Thus, in some examples, the subject has a SARS-CoV-2 infection. In one example, the subject has COVID-19, for example, severe COVID-19. In particular, severe COVID-19 often results in ARDS. The methods of the present disclosure can be used to treat or prevent ARDS in a subject suffering from severe COVID-19.

### Acute Respiratory Distress Syndrome (ARDS)

The present disclosure provides, for example, methods of treating or preventing ARDS in a subject.

ARDS is a life-threatening condition characterized by bilateral pulmonary infiltrates, severe hypoxemia, and disruption of the alveolar-capillary membrane barrier (i.e., pulmonary vascular leak), leading to non-cardiogenic pulmonary edema. There is currently no effective pharmacological therapy.

Infectious etiologies, including influenza and coronavirus infection, are leading causes of ARDS. Accordingly, in one example of the present disclosure, the ARDS is associated with an influenza or a coronavirus infection. For example, the ARDS is associated with influenza. In another example, the ARDS is associated with a coronavirus infection, such as a SARS-COV infection. In one example, the ARDS is associated with a SARS-CoV-2 infection.

ARDS is classified according to the Berlin Definition, which includes:
(1) presentation within 1 week of clinical insult or onset of respiratory symptoms;
(2) acute hypoxemic respiratory failure, as determined by a PaO2/FiO2 ratio of 300 mmHg or less on at least 5 cm of continuous positive airway pressure (CPAP) or positive end expiratory pressure (PEEP), where PaO2 is the partial pressure of oxygen in arterial blood and the FiO2 is the fraction of inspired oxygen;
(3) bilateral opacities on lung radiographs not fully explained by effusions, consolidation, or atelectasis; and
(4) edema/respiratory failure not fully explained by cardiac failure or fluid overload.

In one example, the subject has or suffers from ARDS (i.e., the subject satisfies the Berlin definition of ARDS). For example, the subject is in need of treatment (i.e., in need thereof).

In one example, the subject has or suffers from a symptom associated with ARDS. Symptoms associated with ARDS and methods of identifying subjects at risk of developing ARDS will be apparent to the skilled person and/or are described herein. For example, the subject has one or more or all of the following symptoms:
a) a respiratory frequency of greater than 30 breaths per minute;
b) an oxygen saturation (SpO₂) of 93% or less on room air;
c) a ratio of arterial partial pressure of oxygen to fraction of inspired oxygen (PaO₂/FiO₂) of less than 300 mmHg;
d) a SpO₂/FiO₂ ratio of less than 218; and
e) radiographic lung infiltrates in an amount of greater than 50%.

Currently, ARDS is classified as mild, moderate or severe with an associated increased mortality. The severity of ARDS can be categorized according to the Berlin definition as follows:
(i) Mild ARDS: PaO₂/FiO₂ of 200-300 mmHg on at least 5 cm CPAP or PEEP;
(ii) Moderate ARDS: PaO₂/FiO₂ of 100-200 mmHg on at least 5 cm PEEP; and
(iii) Severe ARDS: PaO2/FiO2 of less than or equal to 100 mmHg on at least 5 cm PEEP.

In one example, the ARDS is mild ARDS. In another example, the ARDS is moderate ARDS. In a further example, the ARDS is severe ARDS.

The methods of the present disclosure can, in addition to treatment of existing ARDS, be used to prevent the onset of ARDS. Thus, in one example, the subject does not have ARDS.

### Kits

Another example of the disclosure provides kits containing a self-replicating RNA of the present disclosure useful for the treatment or prevention of a disease or disorder as described above.

Another example of the disclosure provides kits containing a cRNA of the present disclosure useful for the treatment or prevention of a disease or disorder as described above.

In one example, the kit comprises (a) a container comprising a self-replicating RNA optionally in a delivery system and/or a pharmaceutically acceptable carrier or diluent; and (b) a package insert with instructions for treating or preventing a disease or disorder (e.g., influenza, COVID-19 or ARDS) in a subject.

In one example, the kit comprises (a) a container comprising a cRNA optionally in a delivery system and/or a pharmaceutically acceptable carrier or diluent; and (b) a package insert with instructions for treating or preventing a disease or disorder (e.g., influenza, COVID-19 or ARDS) in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the self-replicating RNA and/or the cRNA. The label or package insert indicates that the composition is used for treating a subject eligible for treatment, e.g., one having or predisposed to developing influenza, an influenza virus infection, a SARS-CoV-2 infection, COVID-19 and/or ARDS, with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Generation of the Self-Replicating RNA

DNA templates encoding the self-replicating RNAs were produced in competent Escherichia coli cells that were transformed with a DNA plasmid. Individual bacterial colonies were isolated and the resultant plasmid DNA amplified in E. coli cultures. Following fermentation, the plasmid DNA was isolated using Maxiprep DNA kit and linearized by restriction digest. Restriction enzymes were then removed using phenol/chloroform extraction and ethanol precipitation.

mRNA was made by in vitro transcription from the linearized DNA template using a T7 RNA polymerase. Subsequently, the DNA template was removed by DNase digestion. Enzymatic capping was performed with Cap0 to provide functional mRNA. The resultant mRNA was purified and resuspended in nuclease-free water.

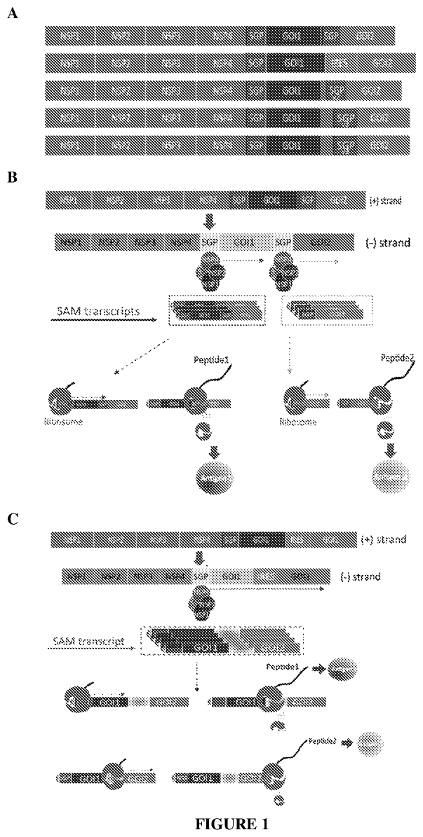

Self-replicating RNAs (FIG. 1A) were prepared using HA and NA subtypes from A/turkey/Turkey/1/2005, NS1 and NP from A/California/2009 and M1 and M2 from PR8X. The following constructs were prepared:
NSP1-4.SGP.H5.SGP.N1 (F548)
NSP1-4.SGP.N1.SGP.H5 (F549)
NSP1-4.SGP.H5.IRES.N1 (F556)
NSP1-4.SGP.N1.IRES.H5 (F557)
NSP1-4.SGP.H5.SGPv2.N1 (F602)
NSP1-4.SGP.H5.SGPv3.N1 (F616)
NSP1-4.SGP.H5.SGPv4.N1 (F617)
NSP1-4.SGP.H5.SGP.N1.SGP.M1 (F554)
NSP1-4.SGP.H5.SGP.NS1 (F568)
NSP1-4.SGP.NS1.SGP.HA (F569)
NSP1-4.SGP.H5.SGP. M1 (F576)
NSP1-4.SGP.M1.SGPv2.NP (F620)
NSP1-4.SGP.M1.SGP.N1.SGP.H5 (F584)
NSP1-4.SGP.M1.SGP.M2.SGP.N1.SGP.H5 (F590)

SGPv2, v3 and v4 were extended at the 5′ with 12, 31 and 52 bases respectively. FIGS. 1B and 1C illustrate the 5′-cap driven antigen expression in the above constructs.

### Example 2: In Vitro Characterisation of the Self-Replicating RNA

The self-replicating RNAs produced in Example 1 were assessed for expression of the first and second genes of interest.

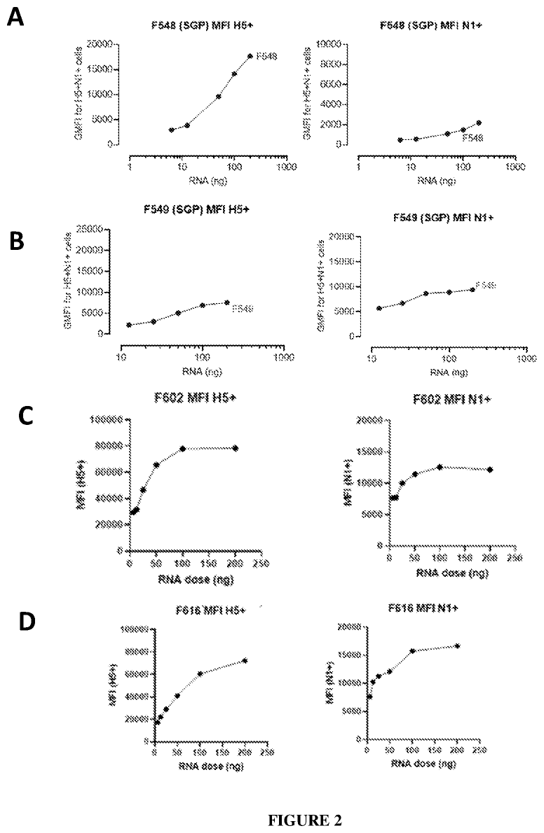

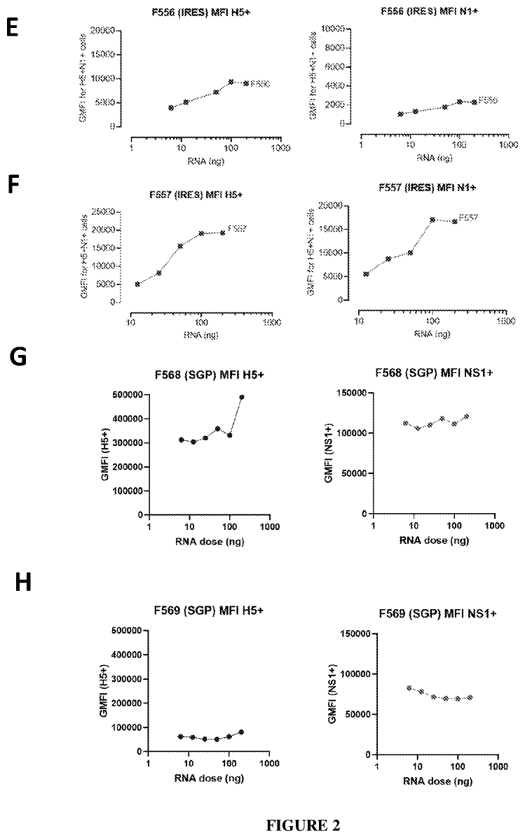

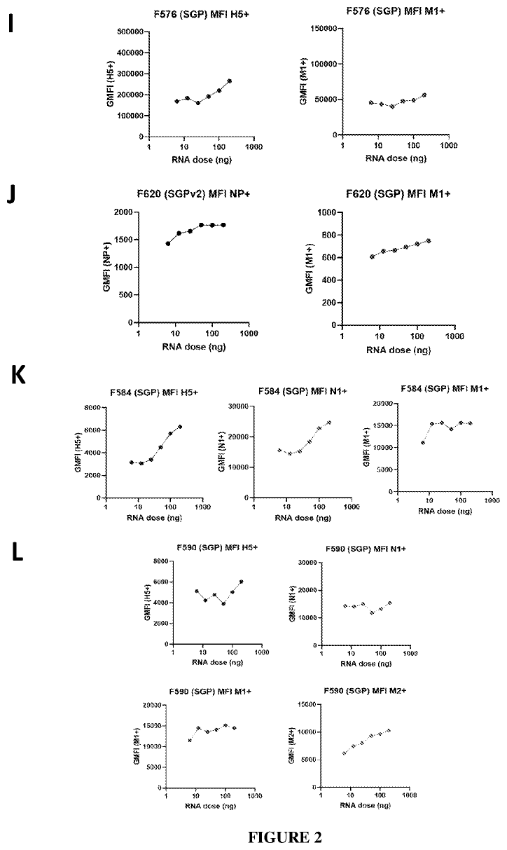

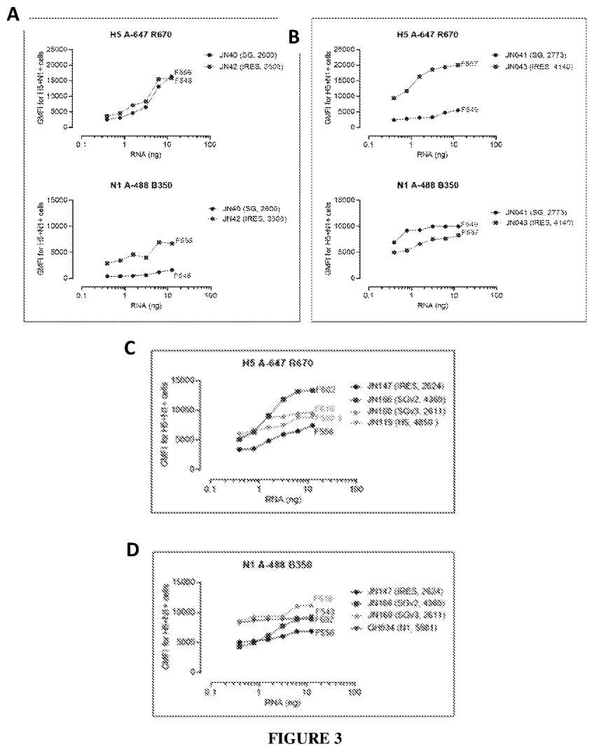

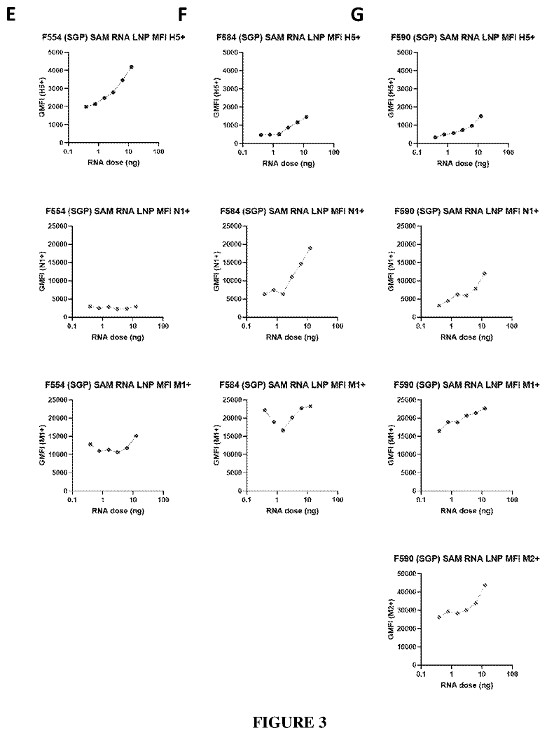

Two-fold serial dilutions of unformulated (naked) or LNP-formulated self-amplifying mRNA constructs were either electroporated or transfected into a Baby Hamster Kidney (BHK) cell line. After 17-19 hrs, cells were harvested and stained for either HA, NA, NS1, NP or M1 antigen expression using anti-HA, anti-NA, anti-NS1, anti-NP or anti-M1 antibodies. The number of cells positive for antigen expression and the mean fluorescence intensities (MFIs) were measured by FACS. Data were analysed to calculate the specific potency values (the probability of successful transfection per unit of mass of RNA) and the MFI of unformulated RNA and LNPs is shown in FIGS. 2 and 3 respectively.

In vitro activity and potency of unformulated RNA and LNPs was determined by FACs based on antigen co-expression and is shown in Table 1 below:

TABLE 1
In vitro activity and potency for unformulated RNA and LNPs

| | FACS | Encapsulation | SAM | | | Zeta | | | |
|---|---|---|---|---|---|---|---|---|---|
| | Potency | Efficiency | recovery | Size | | pot. | Conductivity | Conc. | Endotoxin |
| Vector | (ng⁻¹) | (%) | (%) | (d · nm) | PDI | (mV) | (mS/cm) | (μg/mL) | (EU/mL) |
| F556 | 4439 | 98.3 | 40.6 | 107.9 | 0.048 | 37.7 | 0.0566 | 55.6 | <1 |
| F557 | 5147 | 98.6 | 49.1 | 87.96 | 0.105 | 38 | 0.0611 | 49.8 | <1 |
| F602 | 4360 | 99.2 | 46% | 93.04 | 0.107 | 32 | 0.0577 | 47.8 | <1 |
| F616 | 3902 | 98.3 | 39% | 98.31 | 0.17 | 45.4 | 0.0559 | 35.4 | <1 |

### Antibody Responses

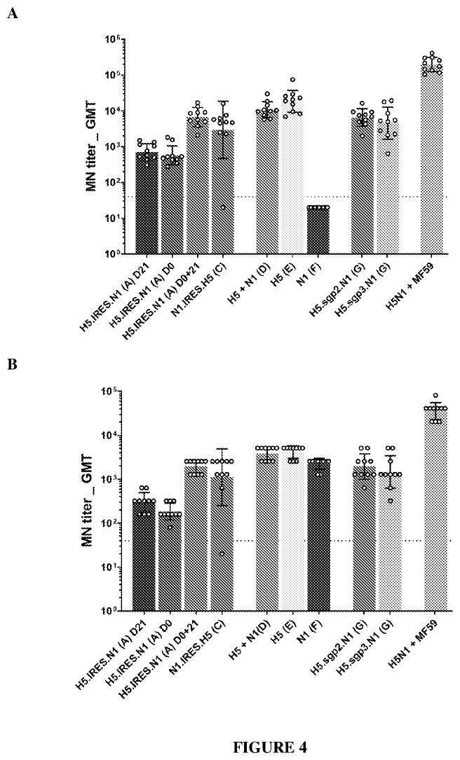

To assess antibody responses, serum was collected at the end of study (i.e., 42 days after first or 21 days after the last, second vaccine dose) and was tested by microneutralization assays (FIG. 4) and hemagglutination inhibition assay (FIG. 5).

For all serological assays sera were treated in the same way, with Vibrio cholerae neuraminidase, also known as receptor-destroying enzyme (RDE) (Denka Seiken Co. Ltd., Tokyo, Japan) and diluted to a starting dilution of 1:10 with PBS. Sheep serum to H5N1 virus (FDA/CBER Kensington lot nu. H5-Ag-1115) was used as positive control sera three assays.

### Microneutralization Assays

Microneutralization assays, short and long form, were performed in a qualified mammalian cell line (proprietary 33016-PF Madin-Darby Canine Kidney (MDCK)).

### Microneutralization Assay Short Form (MNAssay SF)

Virus fluorescent focus-based microneutralization (FFA MN) assay was performed using in house developed protocol. RDE treated test mouse samples and positive control sera were heat inactivated, diluted to a starting dilution of 1:40 with PBS, and fourfold serial diluted using the U-Bottom 96 well plate (BD Falcon) in neutralization medium (comprised of minimum essential medium D-MEM (GIBCO), supplemented with 1% BSA (Rockland, BSA-30), 100 U/mL penicillin and 100 ug/mL streptomycin (GIBCO)). A/turkey/Turkey/1/2005 (H5N1) virus was diluted to ˜ 1,000-1,500 fluorescent focus-forming units (FFU)/well (20,000-30,000 FFU/mL) in neutralization medium and added in a 1:1 ratio to diluted serum.

After incubation for 2 h at 37° C., 5% CO₂, plates (Half Area 96 well plate, Corning) containing MDCK 33016-PF cells were inoculated with this mixture and incubated overnight for 16-18 h at 37° C. with 5% CO₂. MDCK 33016-PF cells had been seeded as 3.0E4/well (3.0E6/plate) at 6-8 h earlier in the cell growth medium (comprised of D-MEM, supplemented with 10% HyClone fetal bovine serum—FBS (Gibco), 100 U/mL penicillin and 100 ug/mL streptomycin). Following the overnight incubation and prior to immunostaining, cells were fixed with cold mixture of acetone and methanol.

The virus was visualized using separate 1 h incubations at room temperature of monoclonal antibodies specific to the nucleoproteins (NP) of the influenza A viruses (clones A1, A3 Blend, Millipore cat. no. MAB8251) and Alexa Fluor 488 Goat Anti-Mouse IgG (H+L) Ab (Invitrogen cat. no. A11001) diluted in PBS buffer containing 0.05% tween-20 (Sigma) and 2% BSA (Fraction V, Calbiochem, 2960, 1194C175). NP viral protein was quantified by a CTL Immunospot analyzer (Cellular Technology Limited, Shaker Heights, Cleveland, OH), using a fluorescein isothiocyanate (FITC) fluorescence filter set with excitation and emission wavelengths of 482 and 536 nm. Fluorescent foci were enumerated by use of software Immunospot 7.0.12.1 professional analyzer DC, using a custom analysis module. The data were successively logged by this software into an Excel data analysis spreadsheet, then 60% focus reduction endpoint was calculated from the average foci count of virus control wells (for each plate), and 60% focus reduction neutralization titer was calculated by linear interpolation between wells immediately above and below the 60% endpoint (for each sample).

### Microneutralization Assay Lone Form (MN Assay LF)

MN assay LF was performed using in house developed protocol. RDE treated test mouse samples and positive control sera were heat inactivated, diluted to a starting dilution of 1:40 with PBS, and twofold serial diluted using the U-Bottom 96 well plate (BD Falcon) in neutralization medium (comprised of the 30% spent growth media (Irvine Scientific) and 70% infective media (protein free media—33016 MDCK PFM; GIBCO) supplemented with 100 U/mL penicillin, 100 ug/mL streptomycin (GIBCO), and 0.33 ug/mL TPCK-trypsin (TPCK treated, Tosyl phenylalanyl chloromethyl ketone, Sigma). A/turkey/Turkey/1/2005 (H5N1) virus was diluted to 100TCID (tissue culture infectious dose) per well in neutralization medium and added in a 1:1 ratio to diluted serum. Serially pre-diluted serum samples are incubated with the virus and allowed to react for 1 h at 37° C., 5% CO₂. In inoculation step, plates (Cell Culture 96-well plate, Costar) containing MDCK 33016-PF cells (which had been seeded as 3.0E4/well (3.0E6/plate) at day before in the antibiotic free cell growth medium (Irvine Scientific) were washed with sterile PBS, then infected with this mixture and incubated for 1 h at 37° C. with 5% CO₂. Infection was stopped by aspiration of antibody/virus mixture and washed cells with sterile PBS are inoculated with neutralizing media (100 ul/well) containing twofold serially diluted antibodies, then incubated for 5 days at 37° C. with 5% CO₂. In the final “read-out” step, detection of virus was performed by HA quantification of the virus using 0.5% turkey red blood cells (Lampire Biological Laboratories). The absence of infectivity constitutes a positive neutralization reaction and indicates the presence of virus-specific antibodies in the serum sample.

### Hemagglutination Inhibition (HAI) Assay

HAI assay was performed as previously described (WHO (2011) Manual for the laboratory diagnosis and virological surveillance of influenza: WHO Press, World Health Organization, 20 Avenue Appia, 1211 Geneva 27, Switzerland). Briefly, RDE treated test mouse samples and positive control sera were heat inactivated, diluted to a starting dilution of 1:10 with PBS, and twofold serial diluted samples (25 μl) were incubated with equal volumes of viruses (4 hemagglutinating units [HAU]) of A/turkey/Turkey/1/2005 (H5N1) at room temperature (RT) for 30 minutes. Then, an equal volume of 0.5% turkey red blood cells (Lampire Biological Laboratories) was added and incubated at RT for 30 minutes. The HAI titer was expressed as the reciprocal of the highest dilution of the samples inhibiting hemagglutination.

### Example 3: Self-Replicating RNA Induces Cell-Mediated Immune Responses

The self-replicating RNAs were assessed for their ability to induce antigen specific T cell responses.

Antigen-specific T cell detection was performed on splenic cultures. Briefly, splenocytes were dissociated in dissociation solution (MACS BSA stock 1:20 with autoMACS rinsing solution) and concentrated at 4E7 cells/ml. Briefly, splenocyte cultures were established in 96 well plates in T cell medium containing RPMI, NEAA, pen/strep and PME) and cultured at 37° C./5% CO₂. Anti-CD28 (clone 37.51; BD Biosciences #553294) and anti-CD107a (clone #1D4B; Biolegend #121618) were added to each well. Cell cultures were either stimulated or unstimulated. To stimulate cultures NA pep mix (JPT Peptide Technologies GmbH; PM-INFA-NATur), HA pep mix (JPT Peptide Technologies GmbH; PM-INFA-HAIndo) was added. Following 2 hours of stimulation, Golgi Plug (with brefeldin A; BD Biosciences #555029) was added to each well. Cells were incubated at 37° C. for a total of 6 hours after which the cells were transferred to 4° C. and stored overnight.

Antigen-specific T cell responses were determined using flow cytometry. Briefly, Fc block mixture (clone 2.4G2; BD Biosciences #553142) was added to each well, followed by extracellular stain (comprising Brilliant stain buffer plus (BD Biosciences #566385), ICOS BV711 (clone C398.4A; Biolegend #313548), CD44 BUV395 (clone IM7; BD Biosciences #740215), CD3 BV786 (clone 145-2C11; BD Biosciences #564379), CD4 APC-H7 (clone GK1.5, BD Biosciences #560181), CD8 AF700 (clone 53-6.7, BD Biosciences #557959) and staining buffer). Cells were stained with UltraComp eBeads (eBiosciences #01-222-42) according to the manufacturer's protocol and incubated at 4° C. for 30 mins, protected from the light. Cells were washed with staining buffer, centrifuged, resuspended in staining buffer and data acquired using a flow cytometer.

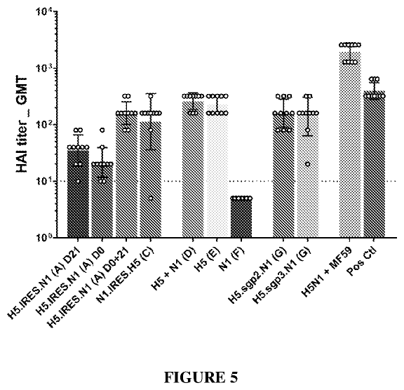

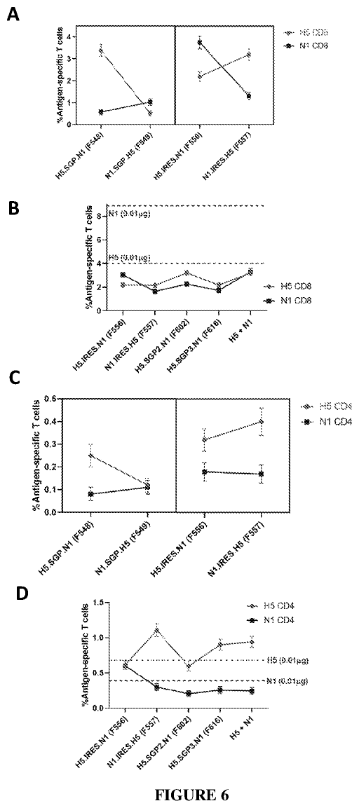

Antigen specific CD4 and CD8 T cell responses are shown in FIG. 6.

### Example 4: Self-Replicating RNAs to SARS-CoV-2

DNA templates encoding the self-replicating RNAs were produced as described in Example 1 above using spike (S) and nucleocapsid (N) antigens from SARS-CoV-2 strain 2019-nCoV/USA-WA1/2020. The following constructs were prepared:
NSP1-4.SGP.S(RRAR→QQAA).SGPv2.N (Col8)
NSP1-4.SGP.S(RRAR→QQAA).SGPv3.N (Co19)

In vitro activity and potency of unformulated RNA and LNPs was determined by FACs based on S and N co-expression and is shown in Table 2 below:

TABLE 2
In vitro activity and potency for unformulated RNA and LNPs

| | FACS | Encapsulation | SAM | | Zeta | | | | Endotoxin | |
|---|---|---|---|---|---|---|---|---|---|---|
| | Potency | Efficiency | Recovery | Size | Pot. | Cond. | | Conc. | Level | Osmolality |
| Vector | (ng−1) | (%) | (%) | (nm) | (mV) | (mS/cm) | PDI | (ug/mL) | (EU/mL) | (mmol/kg) |
| Co18 | 3426 | 97.7 | 53 | 98.28 | 32.7 | 0.0605 | 0.103 | 53.1 | <1 | 291-293 |
| | | | | | | | | | | 295-297 |
| Co19 | 4290 | 99.1 | 65 | 91.09 | 31.1 | 0.0559 | 0.107 | 53.9 | <1 | 290-295 |
| | | | | | | | | | | 292-296 |

### Antibody Responses

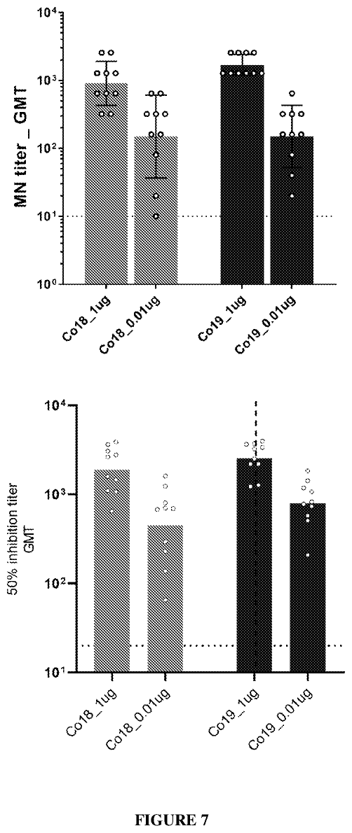

To assess antibody responses, serum was collected at the end of study (i.e., 42 days after first or 21 days after the last, second vaccine dose) and was tested by microneutralization assay as described above in Example 2. The results are shown in FIG. 7A.

Inhibition of ACE2 binding was also assessed. The results are shown in FIG. 7B.

Antibodies specific to the N protein were also assessed by ELISA (Table 3).

TABLE 3
IgG ELISA to N protein

| Vacine | IgG titre (GMT) - 1 μg dose | IgG titre (GMT) - 0.011 μg dose |
|---|---|---|
| Co18 | 30,016 | 2,412 |
| Co19 | 68,290 | 2,947 |

### IgG Subclass

To characterize the type of immune response generated, i.e. Th1 vs Th2 type responses, the S specific IgG1 and IgG2a IgG subclasses were evaluated by ELISA. In addition, the ratio of IgG1/IgG2a antibodies was assessed. Little difference between IgG1 and IgG2a response was observed (Table 4).

TABLE 4
IgG subclasses

| Vector | IgG1 ELISA GMT (1 ug) | IgG2a ELISA GMT (1 ug) |
|---|---|---|
| Co18 | 22,777 | 45,844 |

### Cell-Mediated Immune Responses

The self-replicating RNAs were assessed for their ability to induce antigen specific T cell responses as described in Example 3 above. To stimulate cultures N pep mix (spanning amino acid residues 1-419 of CoV-2 full length N protein), S pep mix 1 (spanning amino acid residues 1-643 of CoV-2 full length S protein), S pep mix 2 (spanning amino acid residues 633-1273 of CoV-2 full length S protein), CoV-1 S peptide (CYGVSATKL) or CoV-2 S peptide (CYGVSPTKL) were added.

Antigen-specific T cell responses were determined using flow cytometry as described above.

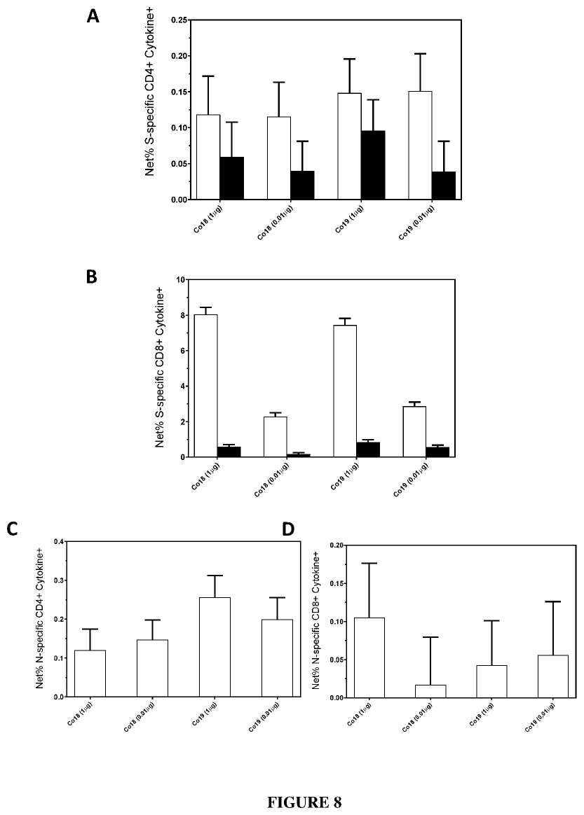

Antigen specific CD4 and CD8 T cell responses are shown in FIG. 8.

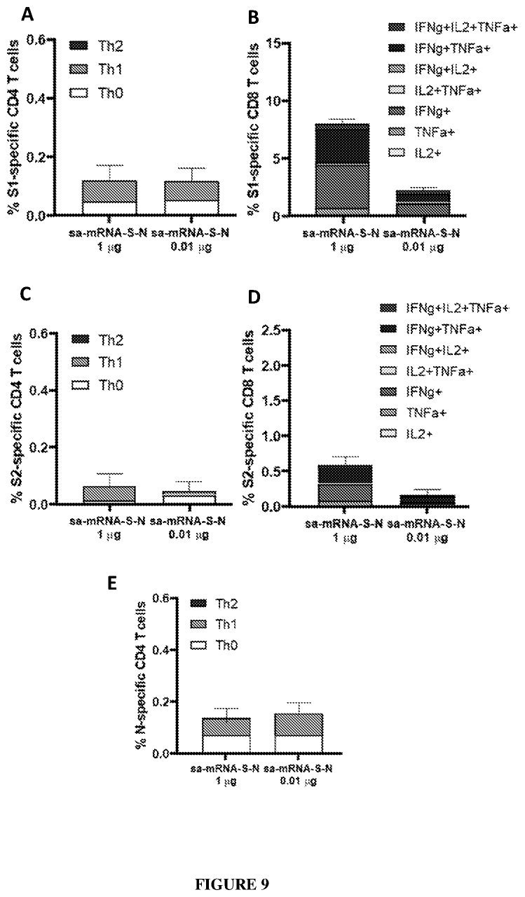

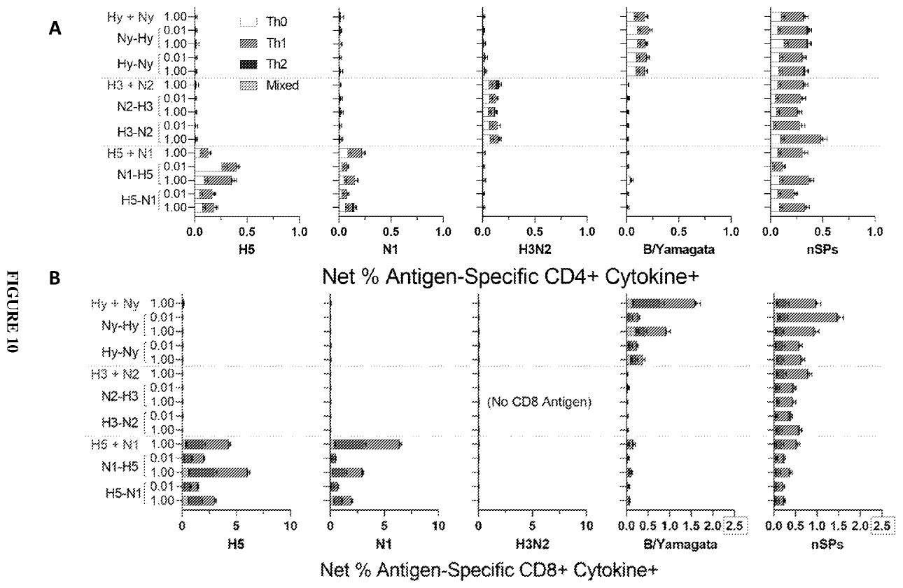

CD4 T cells elicited by sa-mRNA vaccine were mostly Th0 (IL2+ and/or TNFa+, IFNg−, IL5−, IL13−) and Th1 (IFNg+, IL5−, IL13−) with few or no Th2 (IL5+ and/or IL13+, IFNg−) (FIG. 9). Similar frequencies of S1- and S2-reactive CD4 T cells were found; however, for CD8 T cells, S1-reactive T cells dominated over S2-reactive T cells with broad cytokine phenotype, triple, double and single cytokine producing CD8+ T cells.

### Example 5: Protective Effect of Immunization with Self-Replicating RNAs

To evaluate the protective effect of immunization, hamsters were immunized with Col8 at doses of 3 μg RNA/hamster or 0.3 μg RNA/hamster at Day 1 and Day 22. All animals were challenged 28 days post the second immunization with SARS-CoV-2 US virus intranasally and sacrificed 4 days later, when lung and nasal turbinates were collected for infectious virus measured in lungs and nasal turbinates.

In hamsters, 3.0 and 0.3 μg doses raised neutralization titer GMT as 422 and 190 respectively.

To evaluate protection of lungs from virus infection, average virus recovery from lungs were compared for hamsters immunized with Col8 and control hamsters immunized by PBS. While the viral titer from control hamsters was 5,011,872 TCID50/gr., the average virus recovery from vaccine-immunized hamster was under the limit of quantitation of the assay, <20 TCID50/gr. demonstrating the full protection of lower respiratory track with all vaccines included in the study.

To evaluate the protection of upper respiratory track, virus recovery from the nasal turbinates was measured with the average virus recovery from control hamsters of 120,226,443 TCID50/gr. The viral titer was reduced 5×10³ to 10⁴ folds to 14,454 and 21,878 TCID50/gr. for hamsters immunized with Col8 at doses of 3.0 and 0.3 μg respectively. These results demonstrated that sa-mRNA S-N significantly reduced viral infection in upper respiratory track.

### Example 6: Double Dosing with Self-Replicating RNAs to SARS-CoV-2

SARS-CoV-2 S and N antigens are not immunologically cross-reactive. To evaluate the antibody immune response in preclinical animal model, female BALB/c mice were immunized at Day 0 with a dose of 1 g, with a second dose at Day 21. Animals were sacrificed at Day 42 and serum obtained to test for neutralizing antibodies, as well as antibodies inhibiting the binding of S protein to the ACE2 receptor.

In this study, the monocistronic constructs Co6 (comprising an antigen to the SARS-CoV-2 N protein) and Co16 (comprising an antigen to the SARS-CoV-2 S protein) were also used.

The following 1^{st}-2^{nd} a dose combinations were assessed:
PBS-Co18
Co6-Co18
Co16-Co18
Col8-Col8

### S and N Protein Antibodies

Antibodies specific to the S and N proteins were assessed by ELISA at Day 42. The results are shown in Table 5.

TABLE 5
S protein antibodies following prime-boost

| | | IgG titer (GMT) | IgG titer (GMT) |
|---|---|---|---|
| Prime | Boost | to S protein | to N protein |
| PBS | Co18 | 23501 | 13698 |
| Co6 | Co18 | 12882 | 962935 |
| Co16 | Co18 | 499397 | 926 |
| Co18 | Co18 | 991719 | 114006 |
| Co18 | Co6 | 33365 | 5134679 |
| Co18 | Co16 | 856424 | 8961 |
| Co18 | PBS | 28537 | 500 |

Homologous prime/boost (i.e., Col8-Col8) was more effective than heterologous prime-boost with regards an anti-S response, however heterologous prime-boost (i.e., Col8-Co6) was more effective than homologous prime-boost with regards an anti-N response.

In the absence of a boost, anti-S antibodies increased from day 21 to day 42 (data not shown).

### Inhibition of ACE2 Binding

Inhibition of ACE2 binding was also assessed. The results are shown in Table 6.

TABLE 6
Inhibition of ACE2 binding

| | | 50% inhibition of |
|---|---|---|
| Prime | Boost | ACE2 binding (GMT) |
| PBS | Co18 | 468 |
| Co6 | Co18 | 258 |
| Co16 | Co18 | 12195 |
| Co18 | Co18 | 9808 |
| Co18 | Co6 | 872 |
| Co18 | Co16 | 12548 |
| Co18 | PBS | 659 |

### Microneutralization Assay

WT virus neutralization was also assessed. The results are shown in Table 7.

TABLE 7
WT virus neutralisation

| Prime | Boost | MN titer (GMT) |
|---|---|---|
| PBS | Co18 | 43 |
| Co6 | Co18 | 15 |
| Co16 | Co18 | 1493 |
| Co18 | Co18 | 1280 |
| Co18 | Co6 | 92 |
| Co18 | Co16 | 1372 |
| Co18 | PBS | 98 |

### Cell-Mediated Immune Responses

Antigen specific T cell responses were also assessed. CD4 and CD8 T cell responses were observed following vaccination with both homologous and heterologous antigens.

### Example 7: Bicistronic Self-Replicating RNA Vaccines Induce Immune Responses

Bicistronic constructs against influenza were generated as described above in Example 1. The following constructs were generated:
NSP1-4.SGP.H5.SGPv2.N1 (F602)
NSP1-4.SGP.M1.SGPv2.NP (F620)

In vitro activity and potency of the vaccines is shown in Table 7. Bicistronic activity and potency values are based on M1 and NP co-expression for F620 and H5 and N1 co-expression for F602.

TABLE 7
In vitro activity and potency

| Construct | Antigen | RNA Activity (ng⁻¹) | LNP Potency (ng⁻¹) |
|---|---|---|---|
| F620 | M1-NP | 114 | 6040 |
| F602 | H5-N1 | 115 | 5298 |

The vaccine formulations were characterised as shown in Table 8.

TABLE 8
Biophysical characterisation of LNP formulations

| | Potency | Encapsulation | SAM | | Zeta | | | | Endotoxin | |
|---|---|---|---|---|---|---|---|---|---|---|
| | FACS | Efficiency | Recovery | Size | Pot. | Cond. | | Conc. | Level | Osmolality |
| Vector | (ng−1) | (%) | (%) | (nm) | (mV) | (mS/cm) | PDI | (ug/mL) | (EU/mL) | (mmol/kg) |
| F620 | 6040 | 97.2 | 46.8 | 100.4 | 17.5 | 0.0607 | 0.092 | 42.5 | <1 | 292, 296* |
| | | | | | | | | | | 298, 299^{#} |
| F602 | 5298 | 97.5 | 33.2 | 113.6 | 27.2 | 0.0655 | 0.106 | 57.2 | <1 | 294, 296* |
| | | | | | | | | | | 298, 299^{#} |

*first dose;
^{#}second dose

To evaluate the antibody immune response in preclinical animal model, mice were immunized at Day 0 with a dose of 0.1 g or 0.001 g, with a second dose at Day 21. The following 1^{st}-2^{nd} a dose combinations were assessed:

| Prime | Boost |
|---|---|
| F620 | F620 |
| F602 | F602 |
| F620 + F602 | F620 + F602 |

To assess antibody responses, serum was collected at the end of study (i.e., 42 days after first or 21 days after the last, second vaccine dose) and was tested by microneutralization assay as described above in Example 2. All constructs induced virus specific antibodies.

All constructs generated antibodies specific to the N protein as assessed by ELISA.

Antigen specific T cell responses were also assessed. NP and M1 CD4 and NP CD8 T cell specific responses were observed following vaccination. M1 CD8 specific T cell responses were limited.

### Example 8: Regulation of the Second Antigen of Interest by SGP and IRES

Antigen-specific T cells were assessed when the antigen of interest was regulated by the inserted spgv2 promoter or the IRES element.

The following constructs were assessed:
NSP1-4.SGP.H5.IRES.N1 (F556)
NSP1-4.SGP.N1.IRES.H5 (F557)
NSP1-4.SGP.H5.SGPv2.N1 (F602)
NSP1-4.SGP.N1.SGPv2.H5 (F632)

These constructs were compared to the monocistronic N1 and H5 constructs under the control of the native SGP.

Briefly, BALB/c mice vaccinated twice (days 0 and 21) and euthanized on day 42 (3 wks post 2nd vacc). Spleens collected, pooled and assayed for antigen-specific CD4 and CD8 T cells, using an in vitro antigen stimulation/intracellular cytokines immunofluorescence flow cytometry assay. The % CD4 or CD8 T cells that produced cytokines (one or more of IL-2, IFN-g, TNF-a, IL-5, IL-13) were quantified.

As shown in Tables 9 and 10, H5-specific CD8 and CD4 T cells respectively were observed at both doses and with both regulatory elements. It appeared that 0.01 mcg RNA was enough to induce that maximal H5-specific CD4 frequency.

TABLE 9
H5-specific CD8 T cells in BALB/c mouse
spleen 3 weeks post 2nd vaccination

% H5-specific CD8 T cells
(±95% confidence half interval^{b})

| sa-mRNA vaccine | 1 mcg RNA dose | 0.01 mcg RNA dose |
|---|---|---|
| H5.sgpv2.N1 (F602) | 4.77 ± 0.34 | 1.68 ± 0.23 |
| N1.sgpv2.H5 (F632) | 5.43 ± 0.36 | 2.74 ± 0.28 |
| H5.ires.N1 (F556) | 3.87 ± 0.33 | 1.21 ± 0.19 |
| N1.ires.H5 (F557) | 6.84 ± 0.42 | 4.17 ± 0.32 |
| H5 (F500.3) | 9.71 ± 0.48 | 3.83 ± 0.31 |
| N1 (F543) | 0.09 ± 0.05 | 0.14 ± 0.06 |
| H5 + N1 | 4.33 ± 0.32 | 2.07 ± 0.23 |

^{b}measurement precision

TABLE 10
H5-specific CD4 T cells in BALB/c mouse
spleen 3 weeks post 2nd vaccination

% H5-specific CD4 T cells
(±95% confidence half interval^{b})

| sa-mRNA vaccine | 1 mcg RNA dose | 0.01 mcg RNA dose |
|---|---|---|
| H5.sgpv2.N1 (F602) | 0.57 ± 0.09 | 0.56 ± 0.10 |
| N1.sgpv2.H5 (F632) | 0.53 ± 0.10 | 0.64 ± 0.10 |
| H5.ires.N1 (F556) | 0.57 ± 0.11 | 0.24 ± 0.09 |
| N1.ires.H5 (F557) | 0.56 ± 0.11 | 0.84 ± 0.10 |
| H5 (F500.3) | 0.44 □ 0.10 | 0.48 ± 0.09 |
| N1 (F543) | 0.02 □ 0.06 | 0.00 ± 0.06 |
| H5 + N1 | 0.13 □ 0.09 | 0.26 ± 0.08 |

^{b}measurement precision

As shown in Tables 11 and 12, N1-specific CD8 and CD4 T cells respectively were observed at both doses and with both regulatory elements.

TABLE 11
N1-specific CD8 T cells in BALB/c mouse
spleen 3 weeks post 2nd vaccination

% N1-specific CD8 T cells
(±95% confidence half interval^{b})

| sa-mRNA vaccine | 1 mcg RNA dose | 0.01 mcg RNA dose |
|---|---|---|
| H5.sgpv2.N1 (F602) | 2.35 ± 0.24 | 1.39 ± 0.21 |
| N1.sgpv2.H5 (F632) | 2.23 ± 0.25 | 1.22 ± 0.19 |
| H5.ires.N1 (F556) | 4.10 ± 0.33 | 2.10 ± 0.24 |
| N1.ires.H5 (F557) | 3.10 ± 0.29 | 1.56 ± 0.21 |
| H5 (F500.3) | 0.17 ± 0.10 | 0.10 ± 0.07 |
| N1 (F543) | 16.79 ± 0.59 | 5.26 ± 0.35 |
| H5 + N1 | 8.39 ± 0.43 | 3.84 ± 0.30 |

^{b}measurement precision

TABLE 12
N1-specific CD4 T cells in BALB/c mouse
spleen 3 weeks post 2nd vaccination

% N1-specific CD4 T cells
(±95% confidence half interval^{b})

| sa-mRNA vaccine | 1 mcg RNA dose | 0.01 mcg RNA dose |
|---|---|---|
| H5.sgpv2.N1 (F602) | 0.35 ± 0.08 | 0.30 ± 0.08 |
| N1.sgpv2.H5 (F632) | 0.19 ± 0.08 | 0.18 ± 0.08 |
| H5.ires.N1 (F556) | 0.44 ± 0.10 | 0.16 ± 0.09 |
| N1.ires.H5 (F557) | 0.27 ± 0.09 | 0.17 ± 0.07 |
| H5 (F500.3) | −0.04 ± 0.07 | −0.02 ± 0.06 |
| N1 (F543) | 0.69 ± 0.11 | 0.32 ± 0.08 |
| H5 + N1 | 0.23 ± 0.10 | 0.23 ± 0.08 |

^{b}measurement precision

In summary, for both the sgpv2 constructs (F602 and F632) and the IRES constructs (F556 and F557), higher frequencies of antigen-specific T cells were observed with the antigen of interest was regulated by the inserted spgv2 promoter or the IRES element.

### Example 9: Gene Order in the Bicistronic Construct has Little or No Effect on Frequency of Antigen-Specific CD4 T Cells

The impact of gene order in bicistronic constructs using SGPv2 on immune responses was assessed.

Briefly, the following constructs were prepared as previously described:
NSP1-4.SGP.H5.SGPv2.N1 (F602)
NSP1-4.SGP.N1.SGPv2.H5 (F632)
NSP1-4.SGP.H3.SGPv2.N2 (F629)
NSP1-4.SGP.N2.SGPv2.H3 (F703)
NSP1-4.SGP.Hyam.SGPv2.Nyam (F631)
NSP1-4.SGP.Nyam.SGPv2.Hyam (F695)

H5 and N1 antigens were derived from A/turkey/Turkey/01/2005. H3 and N2 antigens were derived from A/Delaware/39/2019. Hyam and Nyam [B/Yamagata] were derived from B/Singapore/INFTT 16 0610/16 (By).

These constructs were compared to the monocistronic N1 and H5 constructs under the control of the native SGP.

Briefly, BALB/c mice vaccinated twice (days 0 and 21) and euthanized on day 42 (3 wks post 2nd vacc). Spleens collected, pooled and assayed for antigen-specific CD4 and CD8 T cells, using an in vitro antigen stimulation/intracellular cytokines immunofluorescence flow cytometry assay. The % CD4 or CD8 T cells that produced cytokines (one or more of IL-2, IFN-g, TNF-a, IL-5, IL-13) were quantified. Neuralisation and antigen-specific ELISAs were also performed.

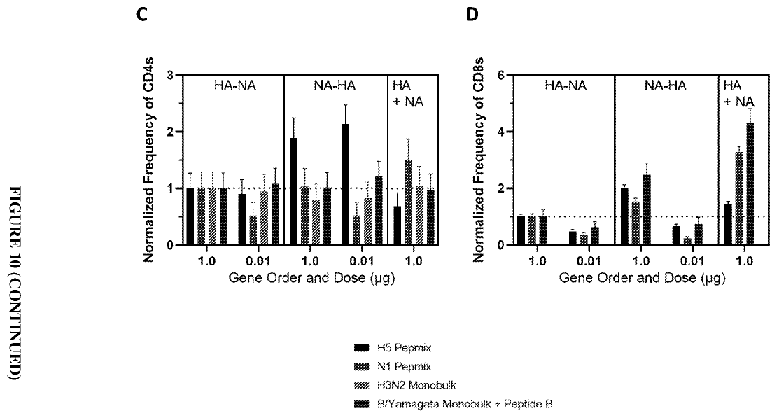

As shown in FIG. 10A, immunization with the sa-mRNA induced a predominantly Th0 or Th1 response. There were few or no mixed responses.

Immunization induced responses to homologous, but not heterologous antigens. Both doses (i.e., 1 μg and 0.01 μg) induced similar CD4 responses.

As shown in FIG. 10B, sa-mRNA vectors induced CD8 responses against matched antigens, with the exception of H3N2.

As shown in FIGS. 10C and D, the NA-HA gene order resulted in a higher frequency CD4 response to H5 and a higher frequency CD8 response to H5, N1 and B/Yamagata.

Serology was also assessed in all constructs in an anti-HA IgG ELISA, hemagglutination inhibition (HAI) assay, pseudovirus microneutralisation assay and anti-NA inhibition assay. All constructs induced a serum antibody response in all assays.

1. A multicistronic self-replicating RNA comprising in order from 5′ to 3′:
a) a first nucleotide sequence encoding a first antigen operably linked to a subgenomic (SG) promoter; and
b) a second nucleotide sequences encoding a second antigen operably linked to a regulatory element selected from the group consisting of a SG promoter and an internal ribosome entry site (IRES).

2. (canceled)

3. The multicistronic self-replicating RNA of claim 1, wherein the RNA comprises one or more additional nucleotide sequences, wherein each sequence encodes an additional antigen operably linked to a regulatory element selected from the group consisting of a SG promoter and an IRES, and wherein the one or more nucleotide sequences are located 3′ of the second nucleotide sequence.

4. The multicistronic self-replicating RNA of claim 1, wherein:
(i) the SG promoter is a minimal SG promoter or an extended SG promoter, wherein the extended SG promoter is extended at the 5′ end with nucleotides occurring in a sequence encoding a non-structural protein of an RNA virus; and/or
(ii) the IRES is a wild-type IRES derived from encephalomyocarditis virus (EMCV).

5. (canceled)

6. (canceled)

7. The multicistronic self-replicating RNA of claim 1, wherein the RNA comprises, in order from 5′ to 3′:
a) a first nucleotide sequence encoding a first antigen operably linked to a minimal SG promoter; and a second nucleotide sequence encoding a second antigen operably linked to a minimal SG promoter; or
b) a first nucleotide sequence encoding a first antigen operably linked to a minimal SG promoter; and a second nucleotide sequence encoding a second antigen operably linked to an extended SG promoter; or
c) a first nucleotide sequence encoding a first antigen operably linked to a minimal SG promoter; and a second nucleotide sequence encoding a second antigen operably linked to a wild-type EMCV IRES.

8. The multicistronic self-replicating RNA of claim 1, wherein:
(i) the minimal SG promoter comprises a sequence set forth in SEQ ID NO: 1;
(ii) the extended SG promoter comprises a sequence set forth in SEQ ID NO: 2 or SEQ ID NO: 3 or SEQ ID NO: 47; and/or
(iii) the wild-type EMCV IRES comprises a sequence set forth in SEQ ID NO: 4.

9-11. (canceled)

12. The multicistronic self-replicating RNA of claim 1, wherein the self-replicating RNA is from an alphavirus, wherein the alphavirus is selected from the group consisting of Semliki Forest virus (SFV), Sindbis virus (SIN), and Venezuelan equine encephalitis virus (VEE), and combinations thereof.

13. (canceled)

14. The multicistronic self-replicating RNA of claim 1, wherein the antigens are viral antigens from a respiratory virus, wherein the respiratory virus is selected from the group consisting of an influenza virus, a respiratory syncytial virus, a parainfluenza virus, a metapneumovirus, a rhinovirus, a coronavirus, an adenovirus, and a bocavirus.

15. (canceled)

16. The multicistronic self-replicating RNA of claim 14, wherein:
(i) the antigens are from different strains of the influenza virus; and/or
(ii) the antigens are from different subtypes of the influenza virus.

17. (canceled)

18. The multicistronic self-replicating RNA of claim 14, wherein:
(i) the antigens are an influenza virus hemagglutinin (HA) protein, a neuraminidase (NA) protein, a matrix (M) protein, a nucleoprotein (NP), and/or a non-structural (NS) protein; and/or
(ii) the antigens are a H5 protein and/or a N1 protein.

19. (canceled)

20. The multicistronic self-replicating RNA of claim 18, wherein the RNA comprises, in order from 5′ to 3′:
a) a first nucleotide sequence encoding the H5 protein; and a second nucleotide sequence encoding the N1 protein; or
b) a first nucleotide sequence encoding the N1 protein; and a second nucleotide sequence encoding the H5 protein.

21. The multicistronic self-replicating RNA of claim 14, wherein the coronavirus is severe acute respiratory syndrome coronavirus 2 (SARS-CoV-2) and wherein the antigens are a SARS-CoV-2 nucleocapsid (N) protein and/or a spike (S) protein.

22. (canceled)

23. The multicistronic self-replicating RNA of claim 21, wherein the RNA comprises, in order from 5′ to 3′:
a) a first nucleotide sequence encoding the N protein; and a second nucleotide sequence encoding the S protein; or
b) a first nucleotide sequence encoding the S protein; and a second nucleotide sequence encoding the N protein.

24. The multicistronic self-replicating RNA of claim 1, wherein the RNA is encoded by a sequence set forth in any one of SEQ ID NO: 10 to 14 or SEQ ID NO: 19 to 27 or SEQ ID NO: 30 to 31.

25. An immunogenic composition comprising the self-replicating RNA of claim 1, comprising a plurality of multicistronic self-replicating RNAs of claim 1, wherein each multicistronic self-replicating RNA encodes different polypeptide antigen sequences.

26. (canceled)

27. A pharmaceutical composition comprising an immunogenic composition of claim 25 and a pharmaceutically acceptable carrier and further comprising a lipid nanoparticle (LNP), a polymeric microparticle or an oil-in-water emulsion, wherein the self-replicating RNA is encapsulated in, bound to, or adsorbed on a LNP, a polymeric microparticle, or an oil-in-water emulsion.

28-32. (canceled)

33. A method of treating or preventing or delaying progression of a disease or condition in a subject or inducing an immune response in a subject, the method comprising administering the immunogenic composition of claim to a subject in need thereof.

34. (canceled)

35. The method of claim 33, wherein:
(i) the disease or condition is a respiratory viral infection, wherein the respiratory viral infection is selected from the group consisting of influenza, an influenza virus infection, bronchiolitis, pneumonia, croup, a SARS-CoV-2 infection, COVID-19, ARDS, and combinations thereof; and/or
(ii) the immune response is a humoral and/or a cell-mediated immune response.

36-39. (canceled)

40. A polynucleotide encoding the self-replicating RNA of claim 1, wherein the polynucleotide is a recombinant DNA, wherein the recombinant DNA is a plasmid.

41-48. (canceled)

49. The multicistronic self-replicating RNA of claim 1, wherein the RNA comprises, in order from 5′ to 3′:
(i) a first nucleotide sequence encoding an influenza virus hemagglutinin (HA) protein operably linked to a SG promoter; and a second nucleotide sequence encoding a neuraminidase (NA) protein operably linked to an extended SG promoter; or
(ii) a first nucleotide sequence encoding a neuraminidase (NA) protein operably linked to a SG promoter; and a second nucleotide sequence encoding an influenza virus hemagglutinin (HA) protein operably linked to an extended SG promoter;
wherein the extended SG promoter is extended at the 5′ end with nucleotides occurring in a sequence encoding a non-structural protein of an RNA virus.

50. The multicistronic self-replicating RNA of claim 1, wherein the RNA comprises, in order from 5′ to 3′:
(i) a first nucleotide sequence encoding an influenza virus hemagglutinin (HA) protein operably linked to a SG promoter comprising a sequence set forth in SEQ ID NO: 1; and a second nucleotide sequence encoding a neuraminidase (NA) protein operably linked to an extended SG promoter comprising a sequence set forth in SEQ ID NO: 2; or
(ii) a first nucleotide sequence encoding a neuraminidase (NA) protein operably linked to a SG promoter comprising a sequence set forth in SEQ ID NO: 1; and a second nucleotide sequence encoding an influenza virus hemagglutinin (HA) protein operably linked to an extended SG promoter comprising a sequence set forth in SEQ ID NO: 2.
